(19) United States
(12) Patent Application Publication
(10) Pub. No.: US 2026/0308694 A1
(43) Pub. Date: Oct. 8, 2026

(54) SYSTEMS AND METHODS RELATING TO PREDICTIVE RIBOSOME BIOGENESIS SCORES FOR IMPROVED CANCER DIAGNOSITICS

(71) Applicants: THE UAB RESEARCH FOUNDATION, BIRMINGHAM, AL (US); The United States Government as Represented by the Department of Veterans Affairs, Washington, DC (US)

(72) Inventors: Lalita SAMANT, VESTAVIA HILLS, AL (US); Amr Rafat ELHAMAMSY, BIRMINGHAM, AL (US); Rajeev Sharad SAMANT, VESTAVIA HILLS, AL (US)

(21) Appl. No.: 19/641,985

(22) Filed: Apr. 8, 2026

Related U.S. Application Data
(60) Provisional application No. 63/785,299, filed on Apr. 8, 2025.

(51) Int. Cl. C12Q 1/6886; G16B 25/10; G16H 10/40; G16H 50/20

(52) U.S. Cl. CPC C12Q 1/6886; G16B 25/10; G16H 10/40; G16H 50/20; C12Q 2600/158

(57) ABSTRACT

Ribosome biogenesis is integral to cell proliferation, and its dysregulation is increasingly recognized as a hallmark of aggressive cancers. To understand these better, various embodiments of the present disclosure develop a score (the RB score) that captures overall ribosome biogenesis activity in tumors. In various examples, higher RB scores can be linked to worse prognostic outcomes. By measuring various genes in ribosome biogenesis, various embodiments of the present disclosure can more accurately predict which tumors are likely to progress faster or respond to immunotherapy. Described herein are kits, methods, and systems relating to determination of a ribosomal biogenesis score. Such a score can be utilized, for example, to determine or otherwise aid in the determination of a disease diagnosis or therapeutic regime, for example, in cancers.

### CROSS-REFERENCE TO RELATED APPLICATIONS

This application claims the benefit of U.S. Provisional Application No. 63/785,299 filed on Apr. 8, 2025, which is incorporated herein by reference in its entirety.

### BACKGROUND

Ribosome biogenesis is the process by which cells create the molecular machinery needed to make proteins. Because cancer cells divide quickly, they often have higher ribosome biogenesis activity, which might drive their aggressiveness and reduce patients' survival. Ribosome biogenesis is integral to cell proliferation, and its dysregulation is increasingly recognized as a hallmark of aggressive cancers. Despite the importance of ribosome biogenesis, the prognostic implications of ribosome biogenesis dysregulation in cancer remain underexplored. Current therapeutic strategies rarely consider the nuances of ribosome biosynthesis in tumors. Accordingly, there is a need to address the aforementioned deficiencies and inadequacies.

### SUMMARY

In an example, a method of identifying a tumor status, comprises detecting an expression level of two or more of a plurality of genes related to ribosomal biogenesis in one or more samples from a subject, wherein the plurality of genes comprises: Variable Charge, X-Linked (VCX), DEAD-Box Helicase 1 (DDX1), Suppressor of Variegation 3-9 Homolog 1 (SUV39H1), DnaJ Heat Shock Protein Family Member C21 (DNAJC21), Ribosome Binding Factor A Homolog (RBFA), Methyltransferase Like 18 (METTL18), Interferon Gamma Inducible Protein 16 (IFI16), Transducin-Like Enhancer Protein 1 (TLE1), Spermatogenesis Associated 5 (SPATA5), Eukaryotic Translation Initiation Factor 2 Subunit Gamma (EIF2S3), Eukaryotic Translation Elongation Factor 1 Alpha 1 (EEF1A1), Mitochondrial Ribosome-Associated GTPase 2 (MTG2), Proliferating Cell Nuclear Antigen (PCNA), Ferredoxin-Dependent Arylamine N-Acetyltransferase-Like Protein (FDXACB1), Ribosomal Protein S27 (RPS27), Mitochondrial Ribosomal Protein S11 (MRPS11), Eukaryotic Translation Initiation Factor 5A-Like 1 (EIF5AL1), Mitochondrial Ribosomal Protein L20 (MRPL20), Mitochondrial Assembly of Ribosomal Large Subunit 1 (MALSU1), Casein Kinase 1 Delta (CSNK1D), Ribosomal Protein S11 (RPS11), Ribosomal Protein S28 (RPS28), Ribosomal Protein S4, X-Linked (RPS4X), Ribosomal Protein L26 Like 1 (RPL26L1), DEAD-Box Helicase 51 (DDX51), RIO Kinase 2 (RIOK2), Ribosomal Protein S17 (RPS17), Ribosomal Protein S15A (RPS15A), Ribosomal Protein S27 Like (RPS27L), Ribosomal Protein S7 (RPS7), Ribosomal Protein L17 (RPL17), Chromosome 18 Open Reading Frame 21 (C18orf21), DNA Damage Inducible Transcript 3 (DDIT3), RNA Terminal Phosphate Cyclase Like 1 (RCL1), Serum Response Factor Binding Protein 1 (SRFBP1), WD Repeat Domain 3 (WDR3), Ribosomal Protein L19 (RPL19), Exosome Component 1 (EXOSC1), Processing of Precursor 4 (POP4), TSR1 Ribosome Maturation Factor (TSR1), Ribosomal RNA Processing 12 (RRP12), UTP14A Small Subunit Processome Component (UTP14A), Eukaryotic Translation Initiation Factor 4A3 (EIF4A3), Processing of Precursor 7 (POP7), Zinc Finger Protein 474 (ZNF474), Transducin Beta Like 3 (TBL3), Nucleolar Complex Associated 3 Homolog (NOC3L), Exosome Component 8 (EXOSC8), TWIST Neighbor (TWISTNB), NOP2 Nucleolar Protein (NOP2), MAK16 Homolog (MAK16), FtsJ RNA Methyltransferase Homolog 3 (FTSJ3), PIN2/TERF1 Interacting Telomerase Inhibitor 1 (PINX1), Zinc Finger HIT-Type Containing 3 (ZNHIT3), Zinc Finger HIT-Type Containing 6 (ZNHIT6), NOP2/Sun RNA Methyltransferase 5 (NSUN5), TATA-Box Binding Protein Associated Factor 1C (TAF1C), PWP1 Homolog (PWP1), DEAD-Box Helicase 31 (DDX31), DEAD-Box Helicase 54 (DDX54), Activator of Basal Transcription 1 (ABT1), Gem Nuclear Organelle Associated Protein 4 (GEMIN4), Cleavage and Polyadenylation Specific Factor 7 (CPSF7), Heterogenous Nuclear Ribonucleoprotein U (HNRNPU), Nucleolar Protein 9 (NOL9), Midasin AAA ATPase 1 (MDN1), Ribosomal RNA Processing 15 (RRP15), and Helicase, POLQ-Like (HELQ); assigning one or more values to the expression level of individual ones of the plurality of genes; summing the one or more assigned values of individual ones of the plurality of genes; comparing a summed value to a threshold; and assigning the tumor status of the subject based at least in part on a comparison of the summed value to the threshold. In examples, the tumor status is an immune infiltration status, a stemness status, a reduced overall survival, or a reduced progression-free interval. In examples, the subject has or is suspected of having one or more of hepatocellular carcinoma (LIHC), kidney renal clear cell carcinoma (KIRC), a breast cancer, lung adenocarcinoma (LUAD), lung squamous cell carcinoma (LUSC), non-small cell lung cancer (NSCLC), melanoma, urothelial cancer, bladder cancer, glioblastoma (GBM), colorectal cancer, ovarian cancer, stomach adenocarcinoma, or endometrial cancer. In examples, the method further comprises outputting a report comprising the one or more assigned values, the summed value, the assigned tumor status, or a combination of any thereof. In examples, the subject can be administered a therapy based at least in part on the tumor status assigned to the subject. In examples, the threshold can be a median of the summed value of a plurality of subjects or a quartile of the summed value of the plurality of subjects.

In examples, described herein are non-transitory, computer-readable media. In an examples, a non-transitory, computer-readable medium can comprise machine-readable instructions that, when executed by a processor of a computing device, cause the computing device to at least: detect an expression level of two or more of a plurality of genes related to ribosomal biogenesis in one or more samples from a subject, wherein the plurality of genes comprises Variable Charge, X-Linked (VCX), DEAD-Box Helicase 1 (DDX1), Suppressor of Variegation 3-9 Homolog 1 (SUV39H1), DnaJ Heat Shock Protein Family Member C21 (DNAJC21), Ribosome Binding Factor A Homolog (RBFA), Methyltransferase Like 18 (METTL18), Interferon Gamma Inducible Protein 16 (IFI16), Transducin-Like Enhancer Protein 1 (TLE1), Spermatogenesis Associated 5 (SPATA5), Eukaryotic Translation Initiation Factor 2 Subunit Gamma (EIF2S3), Eukaryotic Translation Elongation Factor 1 Alpha 1 (EEF1A1), Mitochondrial Ribosome-Associated GTPase 2 (MTG2), Proliferating Cell Nuclear Antigen (PCNA), Ferredoxin-Dependent Arylamine N-Acetyltransferase-Like Protein (FDXACB1), Ribosomal Protein S27 (RPS27), Mitochondrial Ribosomal Protein S11 (MRPS11), Eukaryotic Translation Initiation Factor 5A-Like 1 (EIF5AL1), Mitochondrial Ribosomal Protein L20 (MRPL20), Mitochondrial Assembly of Ribosomal Large Subunit 1 (MALSU1), Casein Kinase 1 Delta (CSNK1D), Ribosomal Protein S11 (RPS11), Ribosomal Protein S28 (RPS28), Ribosomal Protein S4, X-Linked (RPS4X), Ribosomal Protein L26 Like 1 (RPL26L1), DEAD-Box Helicase 51 (DDX51), RIO Kinase 2 (RIOK2), Ribosomal Protein S17 (RPS17), Ribosomal Protein S15A (RPS15A), Ribosomal Protein S27 Like (RPS27L), Ribosomal Protein S7 (RPS7), Ribosomal Protein L17 (RPL17), Chromosome 18 Open Reading Frame 21 (C18orf21), DNA Damage Inducible Transcript 3 (DDIT3), RNA Terminal Phosphate Cyclase Like 1 (RCL1), Serum Response Factor Binding Protein 1 (SRFBP1), WD Repeat Domain 3 (WDR3), Ribosomal Protein L19 (RPL19), Exosome Component 1 (EXOSC1), Processing of Precursor 4 (POP4), TSR1 Ribosome Maturation Factor (TSR1), Ribosomal RNA Processing 12 (RRP12), UTP14A Small Subunit Processome Component (UTP14A), Eukaryotic Translation Initiation Factor 4A3 (EIF4A3), Processing of Precursor 7 (POP7), Zinc Finger Protein 474 (ZNF474), Transducin Beta Like 3 (TBL3), Nucleolar Complex Associated 3 Homolog (NOC3L), Exosome Component 8 (EXOSC8), TWIST Neighbor (TWISTNB), NOP2 Nucleolar Protein (NOP2), MAK16 Homolog (MAK16), FtsJ RNA Methyltransferase Homolog 3 (FTSJ3), PIN2/TERF1 Interacting Telomerase Inhibitor 1 (PINX1), Zinc Finger HIT-Type Containing 3 (ZNHIT3), Zinc Finger HIT-Type Containing 6 (ZNHIT6), NOP2/Sun RNA Methyltransferase 5 (NSUN5), TATA-Box Binding Protein Associated Factor 1C (TAF1C), PWP1 Homolog (PWP1), DEAD-Box Helicase 31 (DDX31), DEAD-Box Helicase 54 (DDX54), Activator of Basal Transcription 1 (ABT1), Gem Nuclear Organelle Associated Protein 4 (GEMIN4), Cleavage and Polyadenylation Specific Factor 7 (CPSF7), Heterogenous Nuclear Ribonucleoprotein U (HNRNPU), Nucleolar Protein 9 (NOL9), Midasin AAA ATPase 1 (MDN1), Ribosomal RNA Processing 15 (RRP15), and Helicase, POLQ-Like (HELQ); assign one or more values to the expression level of individual ones of the plurality of genes; sum the one or more assigned values of individual ones of the plurality of genes; compare a summed value to a threshold; and assign the tumor status of the subject based at least in part on a comparison of the summed value to the threshold. In examples, a tumor status is an immune infiltration status, a stemness status, a reduced overall survival, or a reduced progression-free interval. In examples, a subject has or is suspected of having one or more of hepatocellular carcinoma (LIHC), kidney renal clear cell carcinoma (KIRC), a breast cancer, lung adenocarcinoma (LUAD), lung squamous cell carcinoma (LUSC), non-small cell lung cancer (NSCLC), melanoma, urothelial cancer, bladder cancer, glioblastoma (GBM), colorectal cancer, ovarian cancer, stomach adenocarcinoma, or endometrial cancer. In examples, the machine-readable instructions, when executed by the processor, further cause the computing device to at least output a report comprising the one or more assigned values, the summed value, the assigned tumor status, or a combination of any thereof. In examples, the threshold is a median of the summed value of a plurality of subjects or a quartile of the summed value of the plurality of subjects.

Described herein are kits. In examples, a kit for determining a tumor status in one or more samples from a subject, can comprise a plurality of primer pairs for determining expression levels of two or more of a plurality of genes, wherein the plurality can comprise Variable Charge, X-Linked (VCX), DEAD-Box Helicase 1 (DDX1), Suppressor of Variegation 3-9 Homolog 1 (SUV39H1), DnaJ Heat Shock Protein Family Member C21 (DNAJC21), Ribosome Binding Factor A Homolog (RBFA), Methyltransferase Like 18 (METTL18), Interferon Gamma Inducible Protein 16 (IFI16), Transducin-Like Enhancer Protein 1 (TLE1), Spermatogenesis Associated 5 (SPATA5), Eukaryotic Translation Initiation Factor 2 Subunit Gamma (EIF2S3), Eukaryotic Translation Elongation Factor 1 Alpha 1 (EEF1A1), Mitochondrial Ribosome-Associated GTPase 2 (MTG2), Proliferating Cell Nuclear Antigen (PCNA), Ferredoxin-Dependent Arylamine N-Acetyltransferase-Like Protein (FDXACB1), Ribosomal Protein S27 (RPS27), Mitochondrial Ribosomal Protein S11 (MRPS11), Eukaryotic Translation Initiation Factor 5A-Like 1 (EIF5AL1), Mitochondrial Ribosomal Protein L20 (MRPL20), Mitochondrial Assembly of Ribosomal Large Subunit 1 (MALSU1), Casein Kinase 1 Delta (CSNK1D), Ribosomal Protein S11 (RPS11), Ribosomal Protein S28 (RPS28), Ribosomal Protein S4, X-Linked (RPS4X), Ribosomal Protein L26 Like 1 (RPL26L1), DEAD-Box Helicase 51 (DDX51), RIO Kinase 2 (RIOK2), Ribosomal Protein S17 (RPS17), Ribosomal Protein S15A (RPS15A), Ribosomal Protein S27 Like (RPS27L), Ribosomal Protein S7 (RPS7), Ribosomal Protein L17 (RPL17), Chromosome 18 Open Reading Frame 21 (C18orf21), DNA Damage Inducible Transcript 3 (DDIT3), RNA Terminal Phosphate Cyclase Like 1 (RCL1), Serum Response Factor Binding Protein 1 (SRFBP1), WD Repeat Domain 3 (WDR3), Ribosomal Protein L19 (RPL19), Exosome Component 1 (EXOSC1), Processing of Precursor 4 (POP4), TSR1 Ribosome Maturation Factor (TSR1), Ribosomal RNA Processing 12 (RRP12), UTP14A Small Subunit Processome Component (UTP14A), Eukaryotic Translation Initiation Factor 4A3 (EIF4A3), Processing of Precursor 7 (POP7), Zinc Finger Protein 474 (ZNF474), Transducin Beta Like 3 (TBL3), Nucleolar Complex Associated 3 Homolog (NOC3L), Exosome Component 8 (EXOSC8), TWIST Neighbor (TWISTNB), NOP2 Nucleolar Protein (NOP2), MAK16 Homolog (MAK16), FtsJ RNA Methyltransferase Homolog 3 (FTSJ3), PIN2/TERF1 Interacting Telomerase Inhibitor 1 (PINX1), Zinc Finger HIT-Type Containing 3 (ZNHIT3), Zinc Finger HIT-Type Containing 6 (ZNHIT6), NOP2/Sun RNA Methyltransferase 5 (NSUN5), TATA-Box Binding Protein Associated Factor 1C (TAF1C), PWP1 Homolog (PWP1), DEAD-Box Helicase 31 (DDX31), DEAD-Box Helicase 54 (DDX54), Activator of Basal Transcription 1 (ABT1), Gem Nuclear Organelle Associated Protein 4 (GEMIN4), Cleavage and Polyadenylation Specific Factor 7 (CPSF7), Heterogenous Nuclear Ribonucleoprotein U (HNRNPU), Nucleolar Protein 9 (NOL9), Midasin AAA ATPase 1 (MDN1), Ribosomal RNA Processing 15 (RRP15), and Helicase, POLQ-Like (HELQ); and instructions for use.

In examples, described herein are systems. In examples, a system for determining a tumor status in one or more samples from a subject, can comprise a computing device comprising a processor and a memory; and machine-readable instructions stored in the memory that, when executed by the processor, cause the computing device to at least: identify an expression level of two or more of a plurality of genes related to ribosomal biogenesis in the one or more samples from the subject, wherein the plurality of genes comprises: Variable Charge, X-Linked (VCX), DEAD-Box Helicase 1 (DDX1), Suppressor of Variegation 3-9 Homolog 1 (SUV39H1), DnaJ Heat Shock Protein Family Member C21 (DNAJC21), Ribosome Binding Factor A Homolog (RBFA), Methyltransferase Like 18 (METTL18), Interferon Gamma Inducible Protein 16 (IFI16), Transducin-Like Enhancer Protein 1 (TLE1), Spermatogenesis Associated 5 (SPATA5), Eukaryotic Translation Initiation Factor 2 Subunit Gamma (EIF2S3), Eukaryotic Translation Elongation Factor 1 Alpha 1 (EEF1A1), Mitochondrial Ribosome-Associated GTPase 2 (MTG2), Proliferating Cell Nuclear Antigen (PCNA), Ferredoxin-Dependent Arylamine N-Acetyltransferase-Like Protein (FDXACB1), Ribosomal Protein S27 (RPS27), Mitochondrial Ribosomal Protein S11 (MRPS11), Eukaryotic Translation Initiation Factor 5A-Like 1 (EIF5AL1), Mitochondrial Ribosomal Protein L20 (MRPL20), Mitochondrial Assembly of Ribosomal Large Subunit 1 (MALSU1), Casein Kinase 1 Delta (CSNK1D), Ribosomal Protein S11 (RPS11), Ribosomal Protein S28 (RPS28), Ribosomal Protein S4, X-Linked (RPS4X), Ribosomal Protein L26 Like 1 (RPL26L1), DEAD-Box Helicase 51 (DDX51), RIO Kinase 2 (RIOK2), Ribosomal Protein S17 (RPS17), Ribosomal Protein S15A (RPS15A), Ribosomal Protein S27 Like (RPS27L), Ribosomal Protein S7 (RPS7), Ribosomal Protein L17 (RPL17), Chromosome 18 Open Reading Frame 21 (C18orf21), DNA Damage Inducible Transcript 3 (DDIT3), RNA Terminal Phosphate Cyclase Like 1 (RCL1), Serum Response Factor Binding Protein 1 (SRFBP1), WD Repeat Domain 3 (WDR3), Ribosomal Protein L19 (RPL19), Exosome Component 1 (EXOSC1), Processing of Precursor 4 (POP4), TSR1 Ribosome Maturation Factor (TSR1), Ribosomal RNA Processing 12 (RRP12), UTP14A Small Subunit Processome Component (UTP14A), Eukaryotic Translation Initiation Factor 4A3 (EIF4A3), Processing of Precursor 7 (POP7), Zinc Finger Protein 474 (ZNF474), Transducin Beta Like 3 (TBL3), Nucleolar Complex Associated 3 Homolog (NOC3L), Exosome Component 8 (EXOSC8), TWIST Neighbor (TWISTNB), NOP2 Nucleolar Protein (NOP2), MAK16 Homolog (MAK16), FtsJ RNA Methyltransferase Homolog 3 (FTSJ3), PIN2/TERF1 Interacting Telomerase Inhibitor 1 (PINX1), Zinc Finger HIT-Type Containing 3 (ZNHIT3), Zinc Finger HIT-Type Containing 6 (ZNHIT6), NOP2/Sun RNA Methyltransferase 5 (NSUN5), TATA-Box Binding Protein Associated Factor 1C (TAF1C), PWP1 Homolog (PWP1), DEAD-Box Helicase 31 (DDX31), DEAD-Box Helicase 54 (DDX54), Activator of Basal Transcription 1 (ABT1), Gem Nuclear Organelle Associated Protein 4 (GEMIN4), Cleavage and Polyadenylation Specific Factor 7 (CPSF7), Heterogenous Nuclear Ribonucleoprotein U (HNRNPU), Nucleolar Protein 9 (NOL9), Midasin AAA ATPase 1 (MDN1), Ribosomal RNA Processing 15 (RRP15), and Helicase, POLQ-Like (HELQ) assign one or more values to the expression level of individual ones of the plurality of genes; sum the one or more assigned values of individual ones of the plurality of genes; compare a summed value to a threshold; and assign the tumor status of the subject based at least in part on a comparison of the summed value to the threshold. In examples, a tumor status is an immune infiltration status, a stemness status, a reduced overall survival, or a reduced progression-free interval. In examples, the subject has or is suspected of having one or more of hepatocellular carcinoma (LIHC), kidney renal clear cell carcinoma (KIRC), a breast cancer, lung adenocarcinoma (LUAD), lung squamous cell carcinoma (LUSC), non-small cell lung cancer (NSCLC), melanoma, urothelial cancer, bladder cancer, glioblastoma (GBM), colorectal cancer, ovarian cancer, stomach adenocarcinoma, or endometrial cancer. In examples, the machine-readable instructions, when executed by the processor, further cause the computing device to at least output a report comprising the one or more assigned values, the summed value, the assigned tumor status, or a combination of any thereof. In examples, the subject can be administered a therapy based at least in part on the tumor status assigned to the subject. In examples, the threshold is a median of the summed value of a plurality of subjects or a quartile of the summed value of the plurality of subjects. In examples, systems can comprise machine-readable instructions that, when executed by the processor, cause the computing device to identify the expression level further cause the computing device to at least receive expression data from a thermal cycler and an optical detection device; and identify the expression level of one or more of the plurality of genes related to ribosomal biogenesis in the one or more samples from the subject based at least in part on the expression data. In examples, systems can further comprise one or more of a thermal cycler or an optical detection device.

### BRIEF DESCRIPTION OF THE DRAWINGS

Many aspects of the disclosed devices and methods can be better understood with reference to the following drawings. The components in the drawings are not necessarily to scale, emphasis instead being placed upon clearly illustrating the relevant principles. Moreover, in the drawings, like reference numerals designate corresponding parts throughout the several views.

FIGS. 1A-1G show the development and prognostic implications of the ribosome biogenesis (RB) score in accordance with various embodiments of the present disclosure. FIG. 1A is a flowchart illustrating the identification of 541 ribosome biogenesis-associated genes through comprehensive database screening (PubMed, Gene Ontology, GeneCards, UniProt, OMIM, and PANTHER). FIG. 1B shows the frequency of genetic and transcriptomic alterations in the RB gene set across multiple cancer types using cBioPortal, showing proportions of mutations (green), copy number amplifications (red), deep deletions (blue), and mRNA dysregulations (pink and light blue). FIG. 1C is a Kaplan-Meier analysis comparing overall survival between patients whose tumors harbor RB gene alterations and those without, indicating significantly reduced survival in the altered group (log-rank p=5.004e-4). FIG. 1D shows a distribution of the RB score in The Cancer Genome Atlas (TCGA) tumors, highlighting variability in ribosome biogenesis activity across cancer types. FIG. 1E shows a comparison of RB scores among normal tissue, primary tumors, and metastatic lesions, showing a progressive increase with disease stage (p<0.0001). FIG. 1F shows a Kaplan-Meier curve for overall survival in pan-cancer TCGA cohorts, demonstrating worse prognosis for patients with high RB scores (HR=1.369, log-rank p<0.0001). FIG. 1G is a Kaplan-Meier curve for progression-free interval in the same cohort, revealing that a higher RB score is associated with significantly reduced progression-free times (HR=1.274, log-rank p<0.0001).

FIGS. 2A-2D show protein-protein interaction network and functional enrichment analysis of the ribosome biogenesis (RB) gene set in accordance with various embodiments of the present disclosure. FIG. 2A shows a comprehensive protein-protein interaction (PPI) network of 541 RB-associated genes generated in STRING (p<1.0 e-16). Nodes represent individual genes, and edges denote experimentally validated or predicted interactions, forming dense clusters associated with ribosome biogenesis. FIG. 2B shows top Gene Ontology (GO) terms enriched among RB genes, highlighting significant involvement in key processes such as ribosome biogenesis, rRNA processing, and ribonucleoprotein complex assembly (false discovery rate <1×10\hat{ }-30). FIG. 2C shows reactome pathway enrichment analysis, revealing prominent pathways linked to rRNA processing, eukaryotic translation, and viral mRNA translation, underscoring the pivotal role of these RB genes in both canonical and stress-responsive translational pathways. FIG. 2D shows a KEGG pathway enrichment analysis indicating substantial overrepresentation of RB genes in pathways relevant to ribosome biogenesis, spliceosome function, and RNA metabolism. The size of the data points in FIG. 2C and FIG. 2D corresponds to the number of genes mapped to each pathway, and the color gradient indicates the false discovery rate (FDR).

FIGS. 3A-3E show the high ribosome biogenesis (RB) score is associated with poorer outcomes and enhanced stemness in multiple tumor types in accordance with various embodiments of the present disclosure. FIGS. 3A-3C show Kaplan-Meier survival curves for patients with hepatocellular carcinoma (LIHC), kidney renal clear cell carcinoma (KIRC), and lung adenocarcinoma (LUAD) in the TCGA. Patients were stratified into “High” (red) and “Low” (blue) RB score groups by median RB value. Hazard ratios (HR) and log-rank p-values indicate a significantly worse prognosis for patients with a high RB score. FIG. 3D shows scatter plots showing the relationship between RB score and tumor microenvironment purity (Stromal Score, ESTIMATE Score) in LIHC, KIRC, and LUAD. Pearson's correlation coefficients (r) and p-values suggest that elevated RB scores correlate with reduced tumor purity. FIG. 3E shows scatter plots illustrating a positive correlation between RB score and cancer stemness metrics (e.g., RNA expression-based stemness scores), indicating that tumors with higher RB activity also tend to exhibit more pronounced stemness characteristics.

FIGS. 4A-41 show the development and validation of the Samant RB Score in breast cancer cohorts in accordance with various embodiments of the present disclosure. FIG. 4A is a schematic of the stepwise approach used to develop the Samant RB Score from GSE202203 data: (1) data preprocessing, (2) LASSO feature selection and gene directionality determination, (3) computation of a composite ±1 score, (4) forward selection to maximize R², and (5) derivation of the final 68-gene “Samant RB Score.” FIGS. 4B and 4C show Kaplan-Meier analyses in the GSE202203 cohort, showing that higher Samant RB Scores (red) are significantly associated with poorer overall survival (FIG. 4B) and relapse-free survival (FIG. 4C). Hazard ratios (HR) and log-rank p-values are indicated. FIGS. 4D and 4E show a Samant RB Score distribution by molecular subtype (FIG. 4D) and Nottingham histological grade (FIG. 4E) within GSE202203, demonstrating significant differences among subtypes (Luminal A, Luminal B, HER2+, and Basal) and among histological grades (G1, G2, G3). FIGS. 4F-4H show independent validation in TCGA breast cancer (BRCA) data: higher Samant RB Scores (red) are associated with worse overall survival (FIG. 4F), disease-specific survival (FIG. 4G), and progression-free interval (FIG. 4H). FIG. 4I shows Samant RB Score across major TCGA BRCA subtypes, showing again that basal breast cancers (the most aggressive subtype) have distinct score distributions compared to other subtypes. All survival curves were compared via log-rank tests, and boxplots were analyzed using one-way ANOVA or equivalent nonparametric tests, with multiple-comparison corrections as indicated.

FIGS. 5A1-5E show the curated Samant RB genes and score compares favorably with established immunotherapy biomarkers and predicts clinical outcomes in patients receiving immune checkpoint inhibitors in accordance with various embodiments of the present disclosure. FIGS. 5A1-5A2 show bar charts from the TIDE web tool showing predictive performance (AUC) for several known immunotherapy biomarkers (e.g., CD8, IFNG, MSI score, TIDE) alongside the curated RB score (red). “Random” serves as a baseline reference. FIGS. 5B and 5C show Kaplan-Meier survival curves generated in KMplot (Immunotherapy module) for patients treated with anti-PD-L1 (FIG. 5B) or anti-PD-1 (FIG. 5C) therapy, stratified by high versus low RB expression. Hazard ratios (HR) and log-rank p-values demonstrate improved overall survival in one of the RB expression groups, indicating a potential predictive role of the RB score. FIGS. 5D and 5E show additional KMplot analyses in urothelial cancer (FIG. 5D) and melanoma cohorts (FIG. 5E), illustrating similarly significant survival differences based on the RB expression level in patients receiving anti-PD-L1 or anti-PD-1 treatments.

FIGS. 6A-6F shows an overall survival across TCGA tumor types stratified by a 541-gene ribosome biogenesis (RB) score. Each panel is titled with the respective tumor type. Patients were divided into “High” (red) and “Low” (blue) RB score groups based on the median RB score in each cancer. Kaplan-Meier curves were generated using Python's lifelines package (alongside pandas, numpy, and matplotlib), and the p-values shown in each panel were calculated by log-rank tests to assess survival differences between the two groups.

FIGS. 7A-7E show disease-specific survival across TCGA tumor types stratified by a 541-gene ribosome biogenesis (RB) score. Each panel is labeled with the corresponding tumor type and compares “High” (red) versus “Low” (blue) RB score groups defined by the median RB value per cancer. Disease-specific survival was evaluated using the Python lifelines package (alongside pandas, numpy, and matplotlib), and p-values in each subplot were derived from log-rank tests. As with FIGS. 6A-6F, the 541-gene RB set underlies the score, reflecting ribosome biogenesis activity in each tumor type.

FIGS. 8A-8E show a disease-free interval across TCGA tumor types stratified by a 541-gene ribosome biogenesis (RB) score. Each panel is labeled with the respective tumor type. Patients were divided into “High” (red) and “Low” (blue) RB score groups according to the median RB value per cancer. Disease-free interval curves were generated using Python's lifelines package (together with pandas, numpy, and matplotlib), with log-rank tests used to determine the p-values shown in each plot. These data emphasize the potential influence of ribosome biogenesis activity on disease-free outcomes across multiple tumor types.

FIGS. 9A-9E show progression-free interval across TCGA tumor types stratified by a 541-gene ribosome biogenesis (RB) score. Each panel is labeled with the specific tumor type, distinguishing “High” (red) versus “Low” (blue) RB score groups according to the median RB score per cancer. Curves were generated using Python's lifelines package (alongside pandas, numpy, and matplotlib), and p-values were obtained from log-rank tests. These data suggest that heightened ribosome biogenesis activity may be associated with altered progression-free outcomes across a range of malignancies.

FIGS. 10A-10D show the RB score correlates with aneuploidy, proliferation, and immune cell profiles in LIHC, KIRC, and LUAD. FIGS. 10A-10C are scatter plots illustrating the relationship between RB scores and three tumor-related parameters in each cancer type (rows): aneuploidy score, proliferation module score, and T-cell module score. Pearson's correlation coefficients (r) and p-values are given in each panel, indicating significant positive or negative associations between RB activity and these genomic or immunological features. FIG. 10D shows a heatmap of Pearson correlation coefficients for RB scores versus immune cell subsets, revealing varying degrees of positive (red) or negative (blue) associations with B cells, T cells, macrophages, dendritic cells, and other immune cell types.

FIGS. 11A-11G show LASSO-based model development and assessment using GSE202203 training data. FIG. 11A shows LASSO coefficient paths plotted against varying values of the regularization parameter (a), illustrating the shrinkage of coefficients as a increases. FIG. 11B shows residuals versus fitted values for the final composite score model, showing the distribution and magnitude of prediction errors. FIG. 11C shows summary statistics for the final composite score model, including R-squared, intercept, slope, and mean squared error (MSE). FIG. 11D shows SHAP value distribution for the composite score, indicating how high or low score values contribute to the model's overall output. FIG. 11E shows a calibration plot of the logistic regression classifier on the training set, comparing predicted probabilities to observed outcomes across deciles. FIG. 11F shows Kaplan-Meier survival curves for patients stratified by high (blue) versus low (orange) composite RB score, demonstrating distinct survival trajectories in the training cohort. FIG. 11G is a horizontal bar chart of nonzero LASSO coefficients, sorted by magnitude, with negative coefficients in red and positive coefficients in blue, identifying genes whose expression strongly influences survival predictions.

FIGS. 12A-12F show a protein-protein interaction (PPI) network of the 68-gene Samant RB Score and quartile-based survival analyses in GSE202203 and TCGA BRCA. FIG. 12A shows a PPI network of the 68 ribosome biogenesis-associated genes comprising the Samant RB Score, generated using STRING with a PPI enrichment p<1.0e-16. Nodes represent individual genes, and edges indicate experimentally validated or predicted interactions, forming a dense cluster of RB genes. FIGS. 12B and 12C show Kaplan-Meier curves for overall survival (FIG. 12B) and relapse-free survival (FIG. 12C) in the GSE202203 cohort, comparing patients in the top quartile (red) vs. bottom quartile (blue) of the Samant RB Score. Hazard ratios (HRs) and log-rank p-values demonstrate significantly worse survival outcomes for those with higher RB scores. FIGS. 12D-12F show similar quartile-based analyses in TCGA breast cancer (BRCA) data, showing that high Samant RB Score patients (red) also have poorer disease-specific survival (FIG. 12D), progression-free interval (FIG. 12E), and overall survival (FIG. 12F) compared to the low-score group (blue). All survival comparisons were performed using log-rank tests, and the final cohorts for each comparison are noted on the plots.

FIGS. 13A-13E show Kaplan-Meier plots from TIDE analyses of multiple immunotherapy cohorts, stratified by the Samant RB gene set as a custom biomarker. FIGS. 13A-13E show overall survival curves for patients receiving anti-PD-1 or anti-PD-L1 treatments in the Liu2019, Gide2019, Miao2018, Mariathasan2018, and Zhao2019 datasets, respectively. Each plot was generated using the TIDE platform, in which “Custom Top” versus “Custom Bottom” refers to the median-based split of the Samant RB geneset's expression. Z-statistics and p-values are shown for each dataset, highlighting how high versus low Samant RB gene expression may impact survival in these immunotherapy-treated populations.

FIG. 14 is an example of a computing device according to various embodiments of the present disclosure.

FIG. 15 is a flowchart illustrating an example method according to the present disclosure.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Regarding machine hardware, it should be appreciated that any of the components or modules referred to with regards to any of the present invention embodiments discussed herein, may be integrally or separately formed with one another. Further, redundant functions or structures of the components or modules may be implemented. Moreover, the various components may be communicated locally and/or remotely with any user/operator/customer/client or machine/system/computer/processor. Moreover, the various components may be in communication via wireless and/or hardwire or other desirable and available communication means, systems and hardware. Moreover, various components and modules may be substituted with other modules or components that provide similar functions.

Embodiments of the present disclosure will employ, unless otherwise indicated, techniques of genetics, biochemistry, molecular biology, cellular biology, tissue culture, therapeutic administrations and the like.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only and is not intended to be limiting. It is also possible in the present disclosure that steps can be executed in different sequence where this is logically possible.

### Definitions

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described herein.

As used in the specification and the appended claims, the singular forms “a,” “an,” and “the” may include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently disclosed subject-matter.

The term “about”, when used herein in reference to a value, refers to a value that is similar, in context to the referenced value. In general, those skilled in the art, familiar with the context, will appreciate the relevant degree of variance encompassed by “about” in that context, for example, ±5%, ±4%, ±3%, ±2%, etc.

Two events or entities are “associated” with one another, as that term is used herein, if the presence, level and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

As used herein, the term “comparable” refers to two or more agents, entities, situations, sets of conditions, etc., that may not be identical to one another but that are sufficiently similar to permit comparison there between so that one skilled in the art will appreciate that conclusions can reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Those skilled in the art will appreciate that the term “composition”, as used herein, can be used to refer to a discrete physical entity that comprises one or more specified components. In general, unless otherwise specified, a composition can be of any suitable form—e.g., gel, liquid, solid, etc.

A composition or method described herein as “comprising” one or more named elements or steps is open-ended, meaning that the named elements or steps are essential to a particular aspect or embodiment, but other elements or steps can be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and can also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step can be substituted for that element or step.

In this disclosure, “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, “Improved,” “increased” or “reduced,” or grammatically comparable comparative terms, indicate values that are relative to a baseline value or reference measurement. For example, in some embodiments, an assessed value achieved with an agent of interest may be “improved” relative to that obtained or expected in the absence of treatment or with a comparable reference agent or control. Alternatively, or additionally, in some embodiments, an assessed value achieved with an agent of interest may be “improved” relative to that obtained in the same subject or system under different conditions (e.g., prior to or after an event such as administration of an agent of interest), or in a different, comparable subject (e.g., in a comparable subject or system that differs from the subject or system of interest). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

As used herein, the term “encode” refers to principle that DNA can be transcribed into RNA, which can then be translated into amino acid sequences that can form proteins

As used herein, the terms “optional” or “optionally” means that the subsequently described event or circumstance can or cannot occur, and that the description includes instances where said event or circumstance occurs and instances where it does not.

As used herein, “individual”, “organism”, “host”, “subject”, and “patient” refers to any living entity comprised of at least one cell. A living organism can be as simple as, for example, a single isolated eukaryotic cell or cultured cell or cell line, or as complex as a mammal, including a human being, and animals (e.g., vertebrates, amphibians, fish, mammals, e.g., cats, dogs, horses, pigs, cows, sheep, rodents, rabbits, squirrels, bears, primates (e.g., chimpanzees, gorillas, and humans). These terms (“individual,” “subject,” “host,” and “patient,” used interchangeably herein also refer to any mammalian subject for whom diagnosis, treatment, or therapy is desired, particularly humans. In embodiments, subject may relate to particular components of the subject, for instance specific tissues or fluids of a subject (e.g., human tissue in a particular area of the body of a living subject), which may be in a particular location of the subject, referred to herein as an “area of interest” or a “region of interest.”

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents and are meant to include future updates.

Reference throughout this specification to “one embodiment”, “an embodiment”, “another embodiment”, “some embodiment,” means that a particular feature, structure or characteristic described in connection with the embodiment is included in at least one embodiment of the present invention. Thus, appearances of the phrases “in one embodiment,” “in an embodiment,” “in another embodiment”, or “in some embodiment” in various places throughout this specification are not necessarily all referring to the same embodiment, but they may. Furthermore, the particular features, structures or characteristics may be combined in any suitable manner, as would be apparent to a person skilled in the art from this disclosure, in one or more embodiments. Furthermore, while some embodiments described herein include some, but not other, features included in other embodiments, combinations of features of different embodiments are meant to be within the scope of the invention. For example, in the appended claims, any of the claimed embodiments can be used in any combination.

A “control” sample or value refers to a sample that serves as a reference, usually a known reference, for comparison to a test sample or condition. For example, a test sample can include cells exposed to a test condition or a test agent, while the control is not exposed to the test condition or agent (e.g., negative control). The control can also be a positive control, e.g., a known primary cell or a cell exposed to known conditions or agents, for the sake of comparison to the test condition. A control can also represent an average value gathered from a plurality of samples, e.g., to obtain an average value. For therapeutic applications, a sample obtained from a patient suspected of having a given disorder or deficiency can be compared to samples from a known normal (non-deficient) individual. A control can also represent an average value gathered from a population of similar individuals, e.g., patient having a given deficiency or healthy individuals with a similar medical background, same age, weight, etc. A control value can also be obtained from the same individual, e.g., from an earlier-obtained sample, prior to the disorder or deficiency, or prior to treatment. One of skill will recognize that controls can be designed for assessment of any number of parameters.

The term “biological sample” encompasses a variety of sample types obtained from an organism or a cell line. The term encompasses blood and other liquid samples of biological origin, solid tissue samples, such as a biopsy specimen or tissue cultures or cells derived therefrom and the progeny thereof. The term includes samples that have been manipulated in any way after their procurement, such as by treatment with reagents, solubilization, or enrichment for certain components. The term includes a clinical sample, and includes cells in cell culture, cell supernatants, cell lysates, serum, plasma, biological fluids, and tissue samples. In examples, a biological sample or sample is a tumor biopsy from a subject having a cancer or suspected of having a cancer as described herein.

The term “clinical well-being” as used herein, refers to a state or degree of clinical or physiological wellness or health of a patient. A clinician can evaluate a patient's clinical well-being by physical examination or performing one or more tests or assays.

“Inhibitors,” “activators,” and “modulators” of expression or of activity are used to refer to inhibitory, activating, or modulating molecules, respectively, identified using in vitro and in vivo assays for expression or activity of a described target protein (or encoding polynucleotide), e.g., ligands, agonists, antagonists, and their homologs and mimetics. The term “modulator” includes inhibitors and activators. Inhibitors are agents that, e.g., inhibit expression or bind to, partially or totally block stimulation or protease inhibitor activity, decrease, prevent, delay activation, inactivate, desensitize, or down regulate the activity of the described target protein, e.g., antagonists. Activators are agents that, e.g., induce or activate the expression of a described target protein or bind to, stimulate, increase, open, activate, facilitate, enhance activation or protease inhibitor activity, sensitize or up regulate the activity of described target protein (or encoding polynucleotide), e.g., agonists. Modulators include naturally occurring and synthetic ligands, antagonists and agonists (e.g., small chemical molecules, antibodies and the like that function as either agonists or antagonists). Such assays for inhibitors and activators include, e.g., applying putative modulator compounds to cells expressing the described target protein and then determining the functional effects on the described target protein activity, as described above. Samples or assays comprising described target protein that are treated with a potential activator, inhibitor, or modulator are compared to control samples without the inhibitor, activator, or modulator to examine the extent of effect. Control samples (untreated with modulators) are assigned a relative activity value of 100%. Inhibition of a described target protein is achieved when the activity value relative to the control is about 80%, optionally 50% or 25, 10%, 5% or 1%. Activation of the described target protein is achieved when the activity value relative to the control is 110%, optionally 150%, optionally 200, 300%, 400%, 500%, or 1000-3000% or higher.

The terms “administering,” “delivering,” and “introducing,” can be used interchangeably to indicate the introduction of a therapeutic composition or agent (e.g., compositions comprising one or more therapeutics as described herein) into the body of a subject. The therapeutic composition or agent can be administered through any appropriate means that results in the delivery of at least a portion of the composition or agent to a desired location in the subject such that the composition or agent retains its therapeutic capability. Useful methods of delivering the therapeutic include, but are not limited to, intravenous delivery, subcutaneous delivery, intradermal delivery, intracoronary delivery, intracardiac delivery, oral delivery, or any combination thereof.

The term “therapeutically effective amount” refers to an amount of therapeutic agent effective to treat at least one symptom of a disease or disorder in a subject. In other words, such an amount is sufficient to bring about a beneficial or desired clinical effect. The “therapeutically effective amount” of the agent for administration may vary based upon the desired activity, the diseased state of the subject being treated, the dosage form, method of administration, subject factors such as the subject's sex, genotype, weight and age, the underlying causes of the condition or disease to be treated, the route of administration and bioavailability, the persistence of the administered agent in the body, evidence of natriuresis and/or diuresis, the type of formulation, and the potency of the agent.

As used herein, the terms “pharmaceutically acceptable” or “pharmacologically acceptable” refer to compositions that do not substantially produce adverse reactions, e.g., toxic, allergic, or immunological reactions, when administered to a subject.

The terms “therapy,” “treatment,” and “amelioration” refer to any reduction in the severity of symptoms, e.g., of a neurodegenerative disorder or neuronal injury. As used herein, the terms “treat” and “prevent” are not intended to be absolute terms. Treatment can refer to any delay in onset, amelioration of symptoms, improvement in patient survival, improved cognitive function or coordination, increase in survival time or rate, etc. The effect of treatment can be compared to an individual or pool of individuals not receiving the treatment, or to the same patient prior to treatment or at a different time during treatment. In some aspects, the severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques.

As used throughout, the terms “nucleic acid,” “nucleic acid sequence,” “oligonucleotide,” “nucleotides,” or other grammatical equivalents as used herein mean at least two nucleotides, either deoxyribonucleotides or ribonucleotides, or analogs thereof, covalently linked together. Polynucleotides are polymers of any length, including, e.g., 20, 50, 100, 200, 300, 500, 1000, 2000, 3000, 5000, 7000, 10,000, etc. A polynucleotide described herein generally contains phosphodiester bonds, although in some cases, nucleic acid analogs are included that may have at least one different linkage, e.g., phosphoramidate, phosphorothioate, phosphorodithioate, or O-methylphophoroamidite linkages, and peptide nucleic acid backbones and linkages. Mixtures of naturally occurring polynucleotides and analogs can be made; alternatively, mixtures of different polynucleotide analogs, and mixtures of naturally occurring polynucleotides and analogs may be made. The following are non-limiting examples of polynucleotides: a gene or gene fragment, exons, introns, messenger RNA (mRNA), transfer RNA, ribosomal RNA, ribozymes, cDNA, CRNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, the term polynucleotide encompasses both the double-stranded form and each of two complementary single-stranded forms known or predicted to make up the double-stranded form. A polynucleotide is composed of a specific sequence of four nucleotide bases: adenine (A), cytosine (C), guanine (G), thymine (T), and uracil (U) for thymine when the polynucleotide is RNA. Thus, the term “polynucleotide sequence” is the alphabetical representation of a polynucleotide molecule. Unless otherwise indicated, a particular polynucleotide sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

Unless otherwise indicated, a particular nucleic acid sequence also implicitly encompasses conservatively modified variants thereof, alleles, orthologs, SNPs, and complementary sequences as well as the sequence explicitly indicated.

As used herein, “cDNA” refers to a DNA sequence that is complementary to an RNA transcript in a cell. It is a man-made molecule. Typically, cDNA is made in vitro by an enzyme called reverse-transcriptase using RNA transcripts as templates.

As used herein with reference to the relationship between DNA, cDNA, CRNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

As used herein, “gene” can refer to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989 Molecular Cloning: A Laboratory Manual, 18.1-18.88).

The terms “polypeptide” and “peptide” are used interchangeably herein to refer to a polymer of amino acid residues in a single chain. The terms apply to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L- and D-amino acids. The term “protein” as used herein refers to either a polypeptide or a dimer (i.e., two) or multimer (i.e., three or more) of single chain polypeptides. The single chain polypeptides of a protein may be joined by a covalent bond, e.g., a disulfide bond, or non-covalent interactions. The terms “portion” and “fragment” are used interchangeably herein to refer to parts of a polypeptide, nucleic acid, or other molecular construct.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, y-carboxyglutamate, and O-phosphoserine. Amino acid analogs refer to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The amino acids in the polypeptides described herein can be any of the 20 naturally occurring amino acids, D-stereoisomers of the naturally occurring amino acids, unnatural amino acids and chemically modified amino acids. Unnatural amino acids (that is, those that are not naturally found in proteins) are also known in the art, as set forth in, for example, Zhang et al. “Protein engineering with unnatural amino acids,” Curr. Opin. Struct. Biol. 23(4): 581-87 (2013); Xie et al. “Adding amino acids to the genetic repertoire,” Curr. Opin. Chem. Biol. 9(6): 548-54 (2005); and all references cited therein. Beta and gamma amino acids are known in the art and are also contemplated herein as unnatural amino acids.

In accordance with standard nomenclature, amino acid residue sequences are denominated by either a three letter or a single letter code as indicated as follows, for example: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). “Protein” and “Polypeptide” can refer to a molecule composed of one or more chains of amino acids in a specific order. The term protein is used interchangeable with “polypeptide.” The order is determined by the base sequence of nucleotides in the gene coding for the protein. Proteins can be involved in the structure, function, and regulation of various functions.

The term “identity” or “substantial identity,” as used in the context of a polynucleotide or polypeptide sequence described herein, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 88%, 90%, 91%, 92%, 93%, 94%, 95%, 96%, 97%, 98%, or 99%, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith & Waterman Add. APL. Math. 2:482 (1981), by the homology alignment algorithm of Needleman & Wunsch J. Mol. Biol. 48:443 (1970), by the search for similarity method of Pearson & Lipman Proc. Natl. Acad. Sci. (U.S.A.) 85:2444 (1988), by computerized implementations of these algorithms (e.g., BLAST), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215:403-10 and Altschul et al. (1977) Nucleic Acids Res. 25:3389-402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1977)). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

The terms “co-administration” or “co-administered” as used herein refer to the administration of at least two compounds or agent(s) or therapies to a subject. In some embodiments, the co-administration of two or more agents/therapies is concurrent. In other embodiments, a first agent/therapy is administered prior to a second agent/therapy in this aspect, each component may be administered separately, but sufficiently close in time to provide the desired effect, in particular a beneficial, additive, or synergistic effect. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

The term “composition” as used herein refers to a product comprising the specified ingredients in the specified amounts, as well as any product which results, directly or indirectly, from combination of the specified ingredients in the specified amounts. Such a term in relation to a pharmaceutical composition is intended to encompass a product comprising the active ingredient(s), and the inert ingredient(s) that make up the carrier, as well as any product which results, directly or indirectly, from combination, complexation, or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the present disclosure and a pharmaceutically acceptable carrier.

When a compound of the present disclosure is used contemporaneously with one or more other drugs, a pharmaceutical composition containing such other drugs in addition to the compound of the present disclosure is contemplated. Accordingly, the pharmaceutical compositions of the present disclosure include those that also contain one or more other active ingredients, in addition to a compound of the present disclosure. The weight ratio of the compound of the present disclosure to the second active ingredient may be varied and will depend upon the effective dose of each ingredient. Generally, an effective dose of each will be used. Thus, for example, but not intended to be limiting, when a compound of the present disclosure is combined with another agent, the weight ratio of the compound of the present disclosure to the other agent will generally range from about 1000:1 to about 1:1000, preferably about 200:1 to about 1:200. Combinations of a compound of the present disclosure and other active ingredients will generally also be within the aforementioned range, but in each case, an effective dose of each active ingredient should be used. In such combinations the compound of the present disclosure and other active agents may be administered separately or in conjunction. In addition, the administration of one element may be prior to, concurrent to, or subsequent to the administration of other agent(s).

A composition of the disclosure can be a liquid solution, suspension, emulsion or a powder. Various delivery systems are known and can be used to administer a composition of the disclosure, e.g. encapsulation in liposomes, microparticles, microcapsules, and the like, and then delivered to a patient by means of such as a nebulizer.

Compositions for administration may include sterile aqueous or non-aqueous solvents, such as water, isotonic saline, isotonic glucose solution, buffer solution, or other solvents conveniently used for parenteral administration of therapeutically active agents, stabilizers, buffers, or preservatives, e.g. antioxidants such as methylhydroxybenzoate or similar additives.

A composition of the disclosure may be sterilized by, for example, addition of sterilizing agents to the composition, irradiation of the composition, or heating the composition. Alternatively, the compounds or compositions of the present disclosure may be provided as sterile solid preparations e.g. lyophilized powder, which are readily dissolved in sterile solvent immediately prior to use.

After pharmaceutical compositions have been prepared, they can be placed in an appropriate container and labeled for treatment of an indicated condition. For administration of a composition of the disclosure, such labeling would include amount, frequency, and method of administration.

The term “freeze-dried (lyophilized) as used herein refers to a preparation of primers, for example, that have been initially frozen and the water content removed by vacuum.

The term “pharmaceutically acceptable carrier” as used herein refers to a diluent, adjuvant, excipient, or vehicle with which a probe of the disclosure is administered and which is approved by a regulatory agency of the Federal or a state government or listed in the U.S. Pharmacopeia or other generally recognized pharmacopeia for use in animals, and more particularly in humans. Such pharmaceutical carriers can be liquids, such as water and oils, including those of petroleum, animal, vegetable or synthetic origin, such as peanut oil, soybean oil, mineral oil, sesame oil and the like. The pharmaceutical carriers can be saline, gum acacia, gelatin, starch paste, talc, keratin, colloidal silica, urea, and the like. When administered to a patient, the probe and pharmaceutically acceptable carriers can be sterile. Water is a useful carrier when the probe is administered intravenously. Saline solutions and aqueous dextrose and glycerol solutions can also be employed as liquid carriers, particularly for injectable solutions. Suitable pharmaceutical carriers also include excipients such as glucose, lactose, sucrose, glycerol monostearate, sodium chloride, glycerol, propylene, glycol, water, ethanol and the like. The present compositions, if desired, can also contain minor amounts of wetting or emulsifying agents, or pH buffering agents. The present compositions advantageously may take the form of solutions, emulsion, sustained-release formulations, or any other form suitable for use.

The term “preventing” means to stop or hinder a disease, disorder, or symptom of a disease or condition through some action.

The term “reducing” means to diminish in extent, amount, or degree.

The term “therapeutic agent” as used herein refers to a therapeutic substance selected from a group consisting of, but not limited to immune checkpoint regulators (such as inhibitors), analgesics, anesthetics, anti-inflammatory agents, antiasthma agents, antibiotics (including penicillins), anticoagulants, antihistamines, antitussives, antihypertensive agents, antimuscarinic agents, antimycobacterial agents, antioxidant agents, antipyretics, immunosuppressants, immunostimulants, antiviral agents, bacteriostatic agents, bronchodilators, buffering agents, contrast media, corticosteroids, cough suppressants (expectorants and mucolytics), diagnostic agents, diagnostic imaging agents, free radical scavenging agents, growth factors, haemostatics, immunological agents, lipid regulating agents, muscle relaxants, proteins, peptides and polypeptides, prostaglandins, radio-pharmaceuticals, time release binders, anti-allergic agents, stimulants and anoretics, steroids, sympathomimetics, vasodilators, and xanthines.

The terms “treating” or “treatment” as used herein refers to an alleviation of symptoms associated with a disorder or disease, or inhibition of further progression or worsening of those symptoms, or prevention or prophylaxis of the disease or disorder, or curing the disease or disorder. Similarly, as used herein, an “effective amount” or a “therapeutically effective amount” of a compound of the invention refers to an amount of the compound that alleviates, in whole or in part, symptoms associated with the disorder or condition, or halts or slows further progression or worsening of those symptoms or prevents or provides prophylaxis for the disorder or condition.

### Discussion

### I. Predictive Platforms

Described herein are methods, systems, and machine-readable media relating to determining a tumor status in a subject. In examples, a tumor status can be determined by determining the expression level of two or more of gene products of a plurality of genes associated with ribosomal biogenesis. In examples, the plurality of genes associated with ribosomal biogenesis can be a plurality of 541 genes and their associated gene products (mRNA, for example). In examples, the plurality of genes associated with ribosomal biogenesis can be a plurality of 68 genes and their associated gene products (mRNA, tRNA, rRNA, and/or proteins and fragments thereof, for example). In certain aspects a tumor status can be determined by determining the expression level of any two or more gene products of genes described herein (e.g., mRNA levels of genes described herein).

The methods, systems, and machine-readable media have practical applications in improving the diagnosis of cancers having dysregulated ribosomal biogenesis. The methods, systems, and machine-readable media improve upon existing technology by improving the diagnosis of cancers having dysregulated ribosomal biogenesis by taking into account differential expression of genes related to ribosomal biogenesis-which has previously been overlooked in the field.

In certain aspects, the subject can have or can be suspected of having one or more of hepatocellular carcinoma (LIHC), kidney renal clear cell carcinoma (KIRC), a breast cancer, lung adenocarcinoma (LUAD), lung squamous cell carcinoma (LUSC), non-small cell lung cancer (NSCLC), melanoma, urothelial cancer, bladder cancer, glioblastoma (GBM), colorectal cancer, ovarian cancer, stomach adenocarcinoma, or endometrial cancer. In examples, the subject has or is suspected of having hepatocellular carcinoma (LIHC), kidney renal clear cell carcinoma (KIRC), a breast cancer, or lung adenocarcinoma (LUAD).

In certain aspects, the expression level of the two of more genes can be determined from a biological sample from a subject. In examples, the biological sample can be one or more of a tumor biopsy, a fine needle aspirate (FNA), a core needle biopsy, a formalin-fixed paraffin-embedded (FFPE) tissue sample, a fresh-frozen tissue sample, a surgical resection specimen, a peripheral blood sample, a liquid biopsy sample comprising circulating tumor cells (CTCs), cell-free RNA (cfRNA), or circulating tumor nucleic acids, a pleural effusion sample, an ascites fluid sample, a bronchoalveolar lavage (BAL) sample, or a cerebrospinal fluid (CSF) sample. From the biological sample, gene products of ribosomal biogenesis genes described herein can be isolated (by techniques known in the art, such as RNA extraction techniques and kits, and cDNA synthesis techniques and kits using the extracted RNA), and their expression levels determined. From their determined expression levels, a ribosomal biogenesis score can be determined, and a prognosis can then be assigned from the determined ribosomal biogenesis score. From the assigned prognosis, a healthcare provider can then administer a suitable therapy according to the assigned prognosis, change therapy regimes, and/or administer a different treatment according to the assigned prognosis.

### A. Ribosome Biogenesis-Associated Genes and Gene Products

In the various embodiments of the present disclosure, the plurality of genes related to ribosomal biogenesis can be a panel of 68 genes. As described herein, the panel of 68 genes also includes gene products, for example, messenger RNA whose expression levels can be determined according to methods of the present disclosure.

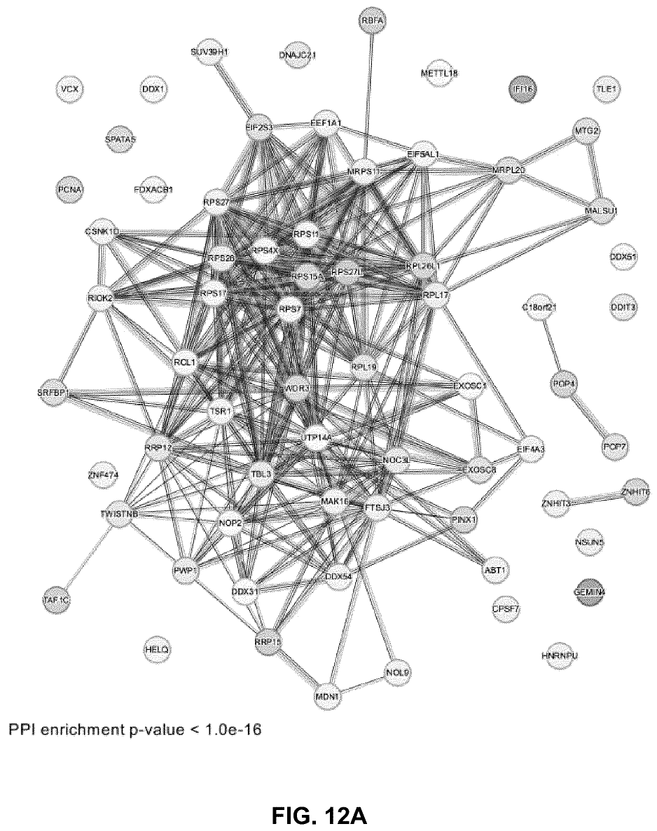

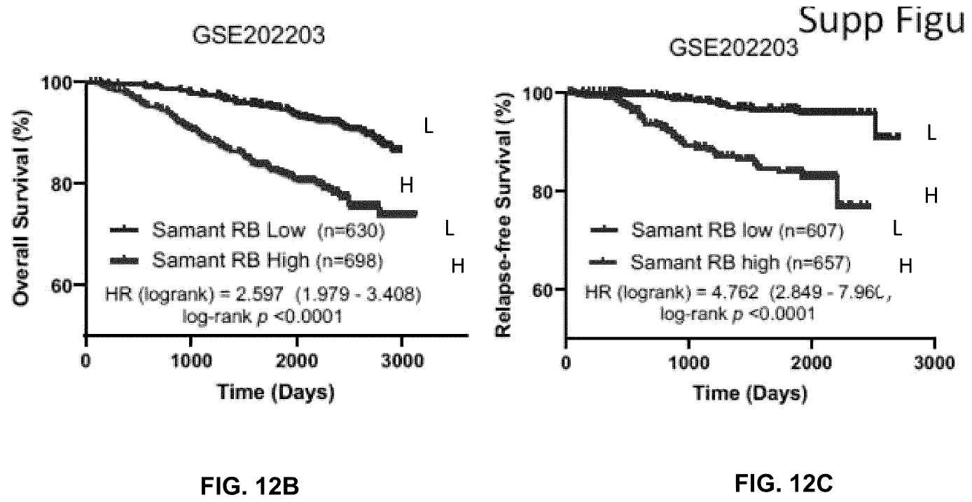

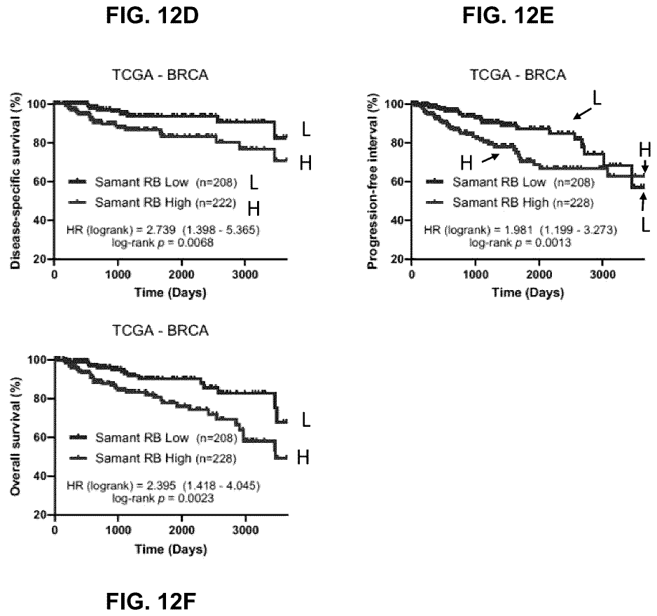

In certain aspects, the expression level of a plurality of any two or more 68 genes listed in FIG. 12A can be determined (or all of them), from a sample from the subject, for example. In examples, the expression level of a plurality of all 68 genes listed in FIG. 12A is determined, from a sample from the subject, for example. The expression levels can have values that can be assigned, for example, based on a mean, median, or average expression level of the same gene in a healthy subject that does not have a cancer. The values assigned to each of the plurality of genes can be summed and the summed value grouped and assigned to a group. From the assignment, a determination of tumor status can be determined.

In certain aspects, the expression level of a plurality of any two or more 68 genes listed in FIG. 12A can include any two or more of: Variable Charge, X-Linked (VCX), DEAD-Box Helicase 1 (DDX1), Suppressor of Variegation 3-9 Homolog 1 (SUV39H1), DnaJ Heat Shock Protein Family Member C21 (DNAJC21), Ribosome Binding Factor A Homolog (RBFA), Methyltransferase Like 18 (METTL18), Interferon Gamma Inducible Protein 16 (IFI16), Transducin-Like Enhancer Protein 1 (TLE1), Spermatogenesis Associated 5 (SPATA5), Eukaryotic Translation Initiation Factor 2 Subunit Gamma (EIF2S3), Eukaryotic Translation Elongation Factor 1 Alpha 1 (EEF1A1), Mitochondrial Ribosome-Associated GTPase 2 (MTG2), Proliferating Cell Nuclear Antigen (PCNA), Ferredoxin-Dependent Arylamine N-Acetyltransferase-Like Protein (FDXACB1), Ribosomal Protein S27 (RPS27), Mitochondrial Ribosomal Protein S11 (MRPS11), Eukaryotic Translation Initiation Factor 5A-Like 1 (EIF5AL1), Mitochondrial Ribosomal Protein L20 (MRPL20), Mitochondrial Assembly of Ribosomal Large Subunit 1 (MALSU1), Casein Kinase 1 Delta (CSNK1D), Ribosomal Protein S11 (RPS11), Ribosomal Protein S28 (RPS28), Ribosomal Protein S4, X-Linked (RPS4X), Ribosomal Protein L26 Like 1 (RPL26L1), DEAD-Box Helicase 51 (DDX51), RIO Kinase 2 (RIOK2), Ribosomal Protein S17 (RPS17), Ribosomal Protein S15A (RPS15A), Ribosomal Protein S27 Like (RPS27L), Ribosomal Protein S7 (RPS7), Ribosomal Protein L17 (RPL17), Chromosome 18 Open Reading Frame 21 (C18orf21), DNA Damage Inducible Transcript 3 (DDIT3), RNA Terminal Phosphate Cyclase Like 1 (RCL1), Serum Response Factor Binding Protein 1 (SRFBP1), WD Repeat Domain 3 (WDR3), Ribosomal Protein L19 (RPL19), Exosome Component 1 (EXOSC1), Processing of Precursor 4 (POP4), TSR1 Ribosome Maturation Factor (TSR1), Ribosomal RNA Processing 12 (RRP12), UTP14A Small Subunit Processome Component (UTP14A), Eukaryotic Translation Initiation Factor 4A3 (EIF4A3), Processing of Precursor 7 (POP7), Zinc Finger Protein 474 (ZNF474), Transducin Beta Like 3 (TBL3), Nucleolar Complex Associated 3 Homolog (NOC3L), Exosome Component 8 (EXOSC8), TWIST Neighbor (TWISTNB), NOP2 Nucleolar Protein (NOP2), MAK16 Homolog (MAK16), FtsJ RNA Methyltransferase Homolog 3 (FTSJ3), PIN2/TERF1 Interacting Telomerase Inhibitor 1 (PINX1), Zinc Finger HIT-Type Containing 3 (ZNHIT3), Zinc Finger HIT-Type Containing 6 (ZNHIT6), NOP2/Sun RNA Methyltransferase 5 (NSUN5), TATA-Box Binding Protein Associated Factor 1C (TAF1C), PWP1 Homolog (PWP1), DEAD-Box Helicase 31 (DDX31), DEAD-Box Helicase 54 (DDX54), Activator of Basal Transcription 1 (ABT1), Gem Nuclear Organelle Associated Protein 4 (GEMIN4), Cleavage and Polyadenylation Specific Factor 7 (CPSF7), Heterogenous Nuclear Ribonucleoprotein U (HNRNPU), Nucleolar Protein 9 (NOL9), Midasin AAA ATPase 1 (MDN1), Ribosomal RNA Processing 15 (RRP15), and Helicase, POLQ-Like (HELQ).

Expression levels of gene products of any two or more (or optionally all) of the plurality described herein can be determined, for example, by extracting RNA from the biological sample, synthesizing cDNA, and amplifying the synthesized cDNA utilizing existing techniques known in the art (PCR-related techniques, for example, such as real-time PCR and quantitative real-time PCR).

### B. Computer (i.e., Machine) Readable Instructions

In various examples, the machine-readable instructions, when executed by the processor, can cause the computing device to at least assign one or more values to a detected expression level of individual ones of the plurality of genes, sum the one or more assigned values of individual ones of the plurality of genes, compare a summed value to a threshold, and assign the tumor status of the subject based at least in part on a comparison of the summed value to the threshold. In some examples, the tumor status can be an immune infiltration status, a stemness status, a reduced overall survival, or a reduced progression-free interval. In examples, such machine-readable instruction can comprise a ribosomal biogenesis score application discussed further in the application below.

In various examples, the machine-readable instructions, when executed by the processor, can cause the computing device to at least detect an expression level of two or more of a plurality of genes related to ribosomal biogenesis in the one or more samples from the subject, assign one or more values to the expression level of individual ones of the plurality of genes, sum the one or more assigned values of individual ones of the plurality of genes, compare a summed value to a threshold, and assign the tumor status of the subject based at least in part on a comparison of the summed value to the threshold. In some examples, the tumor status can be an immune infiltration status, a stemness status, a reduced overall survival, or a reduced progression-free interval.

In further examples, the subject can have or can be suspected of having one or more of hepatocellular carcinoma (LIHC), kidney renal clear cell carcinoma (KIRC), a breast cancer, lung adenocarcinoma (LUAD), lung squamous cell carcinoma (LUSC), non-small cell lung cancer (NSCLC), melanoma, urothelial cancer, bladder cancer, glioblastoma (GBM), colorectal cancer, ovarian cancer, stomach adenocarcinoma, or endometrial cancer. In examples, the subject has or is suspected of having hepatocellular carcinoma (LIHC), kidney renal clear cell carcinoma (KIRC), a breast cancer, or lung adenocarcinoma (LUAD). In some examples, the machine-readable instructions, when executed by the processor, can further cause the computing device to at least output a report comprising the one or more assigned values, the summed value, the assigned tumor status, or a combination of any thereof. In some examples, the subject can be administered a therapy based at least in part on the tumor status assigned to the subject. Further, in various examples, the threshold can be a median of the summed value of a plurality of subjects or a quartile of the summed value of the plurality of subjects.

### C. Systems

Various embodiments of the present disclosure also provide a system for determining a tumor status in one or more samples from a subject, which can include a computing device comprising a processor and a memory, and machine-readable instructions stored in the memory that, when executed by the processor, cause the computing device to at least identify an expression level of one or more of a plurality of genes related to ribosomal biogenesis in the one or more samples from the subject, assign one or more values to the expression level of individual ones of the plurality of genes, sum the one or more assigned values of individual ones of the plurality of genes, compare a summed value to a threshold, and assign the tumor status of the subject based at least in part on a comparison of the summed value to the threshold. In some examples, the tumor status can be an immune infiltration status, a stemness status, a reduced overall survival, or a reduced progression-free interval. In various embodiments of the present disclosure, a system for determining a tumor status in one or more samples from a subject can include one or more of a thermal cycler (for example, a PCR thermal cycler), an optical detection device (one comprising optics that can emit one or more wavelengths of light and detect one or more emitted wavelengths of light, from a fluorophore conjugated to a PCR primer for example), a computing device comprising a processor and a memory, and/or machine-readable instructions stored in the memory.

Further, in some examples, the subject can have or can be suspected of having one or more of hepatocellular carcinoma (LIHC), kidney renal clear cell carcinoma (KIRC), a breast cancer, lung adenocarcinoma (LUAD), lung squamous cell carcinoma (LUSC), non-small cell lung cancer (NSCLC), melanoma, urothelial cancer, bladder cancer, glioblastoma (GBM), colorectal cancer, ovarian cancer, stomach adenocarcinoma, or endometrial cancer, and systems can utilize cDNA synthesized from the sample and determine expression of two or more gene products or a plurality of genes associated with ribosomal-biogenesis described herein. In examples, Ithe subject has or is suspected of having hepatocellular carcinoma (LIHC), kidney renal clear cell carcinoma (KIRC), a breast cancer, or lung adenocarcinoma (LUAD).

In some examples, the machine-readable instructions, when executed by the processor of a system described herein, can further cause the computing device to at least output a report comprising the one or more assigned values, the summed value, the assigned tumor status, or a combination of any thereof. In some examples, the subject can be administered a therapy based at least in part on the tumor status assigned to the subject. In some examples, the threshold can be a median of the summed value of a plurality of subjects or a quartile of the summed value of the plurality of subjects. In further examples, the machine-readable instructions that, when executed by the processor, cause the computing device to identify the expression level can further cause the computing device to at least receive expression data from a thermal cycler and an optical detection device and identify the expression level of one or more of the plurality of genes related to ribosomal biogenesis in the one or more samples from the subject based at least in part on the expression data.

In an example, a system for determining a tumor status in one or more samples from a subject, can comprise: a computing device comprising a processor and a memory; and machine-readable instructions stored in the memory that, when executed by the processor, cause the computing device to at least: identify an expression level of two or more of a plurality of genes related to ribosomal biogenesis in the one or more samples from the subject, wherein the plurality of genes can comprise: VCX; DDX1; SUV39H1; DNAJC21; RBFA; METTL18; IFI16; TLE1; SPATA5; EIF2S3; EEF1A1; MTG2; PCNA; FDXACB1; RPS27; MRPS11; EIF5AL1; MRPL20; MALSU1; CSNK1D; RPS11; RPS28; RPS4X; RPL26L1; DDX51; RIOK2; RPS17; RPS15A; RPS27L; RPS7; RPL17; C18orf21; DDIT3; RCL1; SRFBP1; WDR3; RPL19; EXOSC1; POP4; TSR1; RRP12; UTP14A; EIF4A3; POP7; ZNF474; TBL3; NOC3L; EXOSC8; TWISTNB; NOP2; MAK16; FTSJ3; PINX1; ZNHIT3; ZNHIT6; NSUN5; TAF1C; PWP1; DDX31; DDX54; ABT1; GEMIN4; CPSF7; HNRNPU; NOL9; MDN1; RRP15; and HELQ; assign one or more values to the expression level of individual ones of the plurality of genes; sum the one or more assigned values of individual ones of the plurality of genes; compare a summed value to a threshold; and assign the tumor status of the subject based at least in part on a comparison of the summed value to the threshold. In examples, the tumor status can be an immune infiltration status, a stemness status, a reduced overall survival, or a reduced progression-free interval. In examples, the subject can have or can be suspected of having one or more of hepatocellular carcinoma (LIHC), kidney renal clear cell carcinoma (KIRC), a breast cancer, or lung adenocarcinoma (LUAD). In examples, the subject can have or can be suspected of having one or more of hepatocellular carcinoma (LIHC), kidney renal clear cell carcinoma (KIRC), a breast cancer, lung adenocarcinoma (LUAD), lung squamous cell carcinoma (LUSC), non-small cell lung cancer (NSCLC), melanoma, urothelial cancer, bladder cancer, glioblastoma (GBM), colorectal cancer, ovarian cancer, stomach adenocarcinoma, or endometrial cancer. In examples, the machine-readable instructions, when executed by the processor, can further cause the computing device to at least output a report comprising the one or more assigned values, the summed value, the assigned tumor status, or a combination of any thereof. In examples, the subject can be administered a therapy based at least in part on the tumor status assigned to the subject. In examples, the threshold can be a median of the summed value of a plurality of subjects or a quartile of the summed value of the plurality of subjects. In examples, the machine-readable instructions that, when executed by the processor, can cause the computing device to identify the expression level further cause the computing device to at least: receive expression data from a thermal cycler and an optical detection device; and identify the expression level of one or more of the plurality of genes related to ribosomal biogenesis in the one or more samples from the subject based at least in part on the expression data.

In an example, described herein is system for determining a tumor status in one or more samples from a subject, comprising: a thermal cycler; an optical detection device; a computing device comprising a processor and a memory; and machine-readable instructions stored in the memory that, when executed by the processor, cause the computing device to at least: detect an expression level of two or more of a plurality of genes related to ribosomal biogenesis in the one or more samples from the subject, wherein the plurality of genes can comprise: VCX; DDX1; SUV39H1; DNAJC21; RBFA; METTL18; IFI16; TLE1; SPATA5; EIF2S3; EEF1A1; MTG2; PCNA; FDXACB1; RPS27; MRPS11; EIF5AL1; MRPL20; MALSU1; CSNK1D; RPS11; RPS28; RPS4X; RPL26L1; DDX51; RIOK2; RPS17; RPS15A; RPS27L; RPS7; RPL17; C18orf21; DDIT3; RCL1; SRFBP1; WDR3; RPL19; EXOSC1; POP4; TSR1; RRP12; UTP14A; EIF4A3; POP7; ZNF474; TBL3; NOC3L; EXOSC8; TWISTNB; NOP2; MAK16; FTSJ3; PINX1; ZNHIT3; ZNHIT6; NSUN5; TAF1C; PWP1; DDX31; DDX54; ABT1; GEMIN4; CPSF7; HNRNPU; NOL9; MDN1; RRP15; and HELQ; assign one or more values to the expression level of individual ones of the plurality of genes; sum the one or more assigned values of individual ones of the plurality of genes; compare a summed value to a threshold; and assign the tumor status of the subject based at least in part on a comparison of the summed value to the threshold. In examples, the tumor status can be an immune infiltration status, a stemness status, a reduced overall survival, or a reduced progression-free interval.

In examples, the subject can have or can be suspected of having one or more of hepatocellular carcinoma (LIHC), kidney renal clear cell carcinoma (KIRC), a breast cancer, lung adenocarcinoma (LUAD), lung squamous cell carcinoma (LUSC), non-small cell lung cancer (NSCLC), melanoma, urothelial cancer, bladder cancer, glioblastoma (GBM), colorectal cancer, ovarian cancer, stomach adenocarcinoma, or endometrial cancer. In examples, the subject has or is suspected of having hepatocellular carcinoma (LIHC), kidney renal clear cell carcinoma (KIRC), a breast cancer, or lung adenocarcinoma (LUAD). In examples, the machine-readable instructions, when executed by the processor, further cause the computing device to at least output a report comprising the one or more assigned values, the summed value, the assigned tumor status, or a combination of any thereof. In examples, the subject is administered a therapy based at least in part on the tumor status assigned to the subject. In examples, the threshold is a median of the summed value of a plurality of subjects or a quartile of the summed value of the plurality of subjects.

### D. Computing Systems

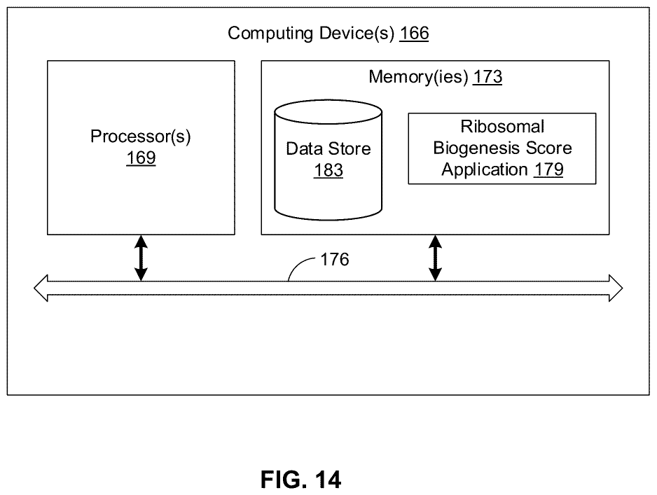

With respect to FIG. 14, shown is a schematic block diagram of one or more computing devices 166. Each computing device 166 includes at least one processor circuit, for example, having a processor 169 and a memory 173, both of which can be coupled to a local interface 176. To this end, each computing device 166 can comprise, for example, at least one server computer or like device. The local interface 176 may comprise, for example, a data bus with an accompanying address/control bus or other bus structure as can be appreciated.

Stored in the memory 173 are both data and several components that are executable by the processor 169. In particular, stored in the memory 173 and executable by the processor 169 can be a ribosomal biogenesis score application 179 and potentially other applications. Also stored in the memory 173 may be a data store 183 and other data. In addition, an operating system may be stored in the memory 173 and executable by the processor 169.

It is understood that there may be other applications that are stored in the memory 173 and are executable by the processor 169 as can be appreciated. Where any component discussed herein is implemented in the form of software, any one of a number of programming languages may be employed such as, for example, C, C++, C#, Objective C, Java®, JavaScript®, Perl, PHP, Visual Basic®, Python®, Ruby, Flash®, or other programming languages.

A number of software components are stored in the memory 173 and are executable by the processor 169. In this respect, the term “executable” means a program file that is in a form that can ultimately be run by the processor 169. Examples of executable programs may be, for example, a compiled program that can be translated into machine code in a format that can be loaded into a random access portion of the memory 173 and run by the processor 169, source code that may be expressed in proper format such as object code that is capable of being loaded into a random access portion of the memory 173 and executed by the processor 169, or source code that may be interpreted by another executable program to generate instructions in a random access portion of the memory 173 to be executed by the processor 169, etc. An executable program may be stored in any portion or component of the memory 173 including, for example, random access memory (RAM), read-only memory (ROM), hard drive, solid-state drive, USB flash drive, memory card, optical disc such as compact disc (CD) or digital versatile disc (DVD), floppy disk, magnetic tape, or other memory components.

The memory 173 is defined herein as including both volatile and nonvolatile memory and data storage components. Volatile components are those that do not retain data values upon loss of power. Nonvolatile components are those that retain data upon a loss of power. Thus, the memory 173 may comprise, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM may comprise, for example, static random-access memory (SRAM), dynamic random-access memory (DRAM), or magnetic random-access memory (MRAM) and other such devices. The ROM may comprise, for example, a programmable read-only memory (PROM), an erasable programmable read-only memory (EPROM), an electrically erasable programmable read-only memory (EEPROM), or other like memory device.

Also, the processor 169 may represent multiple processors 169 and/or multiple processor cores and the memory 173 may represent multiple memories 173 that operate in parallel processing circuits, respectively. In such a case, the local interface 176 may be an appropriate network that facilitates communication between any two of the multiple processors 169, between any processor 169 and any of the memories 173, or between any two of the memories 173, etc. The local interface 176 may comprise additional systems designed to coordinate this communication, including, for example, performing load balancing. The processor 169 may be of electrical or of some other available construction.

Although the ribosomal biogenesis score application 179 and other various systems described herein may be embodied in software or code executed by general purpose hardware as discussed above, as an alternative the same may also be embodied in dedicated hardware or a combination of software/general purpose hardware and dedicated hardware. If embodied in dedicated hardware, each can be implemented as a circuit or state machine that employs any one of or a combination of a number of technologies. These technologies may include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc. Such technologies are generally well known by those skilled in the art and, consequently, are not described in detail herein.

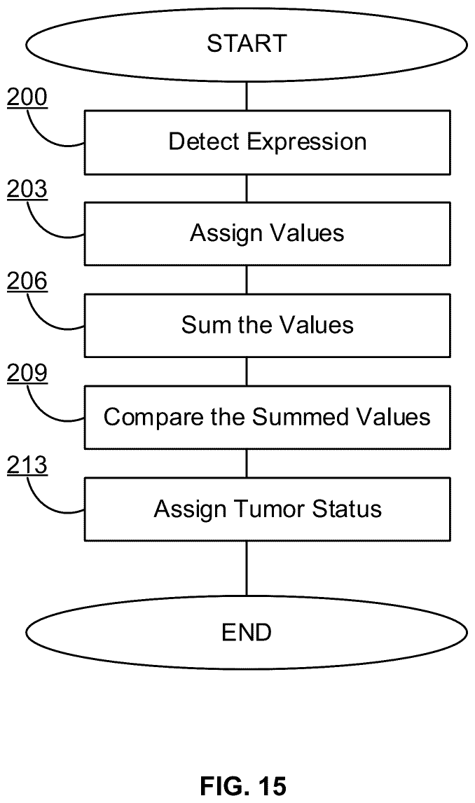

Referring next to FIG. 15, shown is a flowchart that provides one example of the operation of the ribosomal biogenesis score application 179. The flowchart of FIG. 15 provides merely an example of the many different types of functional arrangements that can be employed to implement the operation of the instructions of the application. Alternatively, the flowchart of FIG. 15 could be viewed as depicting a method implemented by the ribosomal biogenesis score application 179.

Beginning at block 200, the ribosomal biogenesis score application 179 can detect the expression level[s] of two or more of any two or more from a plurality of VCX; DDX1; SUV39H1; DNAJC21; RBFA; METTL18; IFI16; TLE1; SPATA5; EIF2S3; EEF1A1; MTG2; PCNA; FDXACB1; RPS27; MRPS11; EIF5AL1; MRPL20; MALSU1; CSNK1D; RPS11; RPS28; RPS4X; RPL26L1; DDX51; RIOK2; RPS17; RPS15A; RPS27L; RPS7; RPL17; C18orf21; DDIT3; RCL1; SRFBP1; WDR3; RPL19; EXOSC1; POP4; TSR1; RRP12; UTP14A; EIF4A3; POP7; ZNF474; TBL3; NOC3L; EXOSC8; TWISTNB; NOP2; MAK16; FTSJ3; PINX1; ZNHIT3; ZNHIT6; NSUN5; TAF1C; PWP1; DDX31; DDX54; ABT1; GEMIN4; CPSF7; HNRNPU; NOL9; MDN1; RRP15; and HELQ. In examples, block 200 can acquire expression data of all of VCX; DDX1; SUV39H1; DNAJC21; RBFA; METTL18; IFI16; TLE1; SPATA5; EIF2S3; EEF1A1; MTG2; PCNA; FDXACB1; RPS27; MRPS11; EIF5AL1; MRPL20; MALSU1; CSNK1D; RPS11; RPS28; RPS4X; RPL26L1; DDX51; RIOK2; RPS17; RPS15A; RPS27L; RPS7; RPL17; C18orf21; DDIT3; RCL1; SRFBP1; WDR3; RPL19; EXOSC1; POP4; TSR1; RRP12; UTP14A; EIF4A3; POP7; ZNF474; TBL3; NOC3L; EXOSC8; TWISTNB; NOP2; MAK16; FTSJ3; PINX1; ZNHIT3; ZNHIT6; NSUN5; TAF1C; PWP1; DDX31; DDX54; ABT1; GEMIN4; CPSF7; HNRNPU; NOL9; MDN1; RRP15; and HELQ.

At block 203, the ribosomal biogenesis score application 179 can assign values to detected or determined expression level[s] of the genes from block 200.

At block 206, the ribosomal biogenesis score application 179 can sum the detected or determined expression level[s] to create a ribosomal biogenesis score.

At block 209, the ribosomal biogenesis score application 179 can compare the summed values from block 206 to a threshold value, for example, a control healthy sample not having a cancer or a subject having a cancer not associated with ribosomal biogenesis or not having a cancer specifically as described herein.

Next, at block 213, the ribosomal biogenesis score application 179 can assign a tumor status based on the comparison from block 209. From the assigned tumor status of block 213, a treatment status can be determined or otherwise updated or changed from an existing treatment.

### II. Kits and Packaging

Described herein are kits comprising two or more sets of primers (i.e., a plurality of primers, for example, polymerase chain reaction (PCR) primers), in addition to containers for storage and/or use, and instructions for use. The kits two or more sets of primers can be utilized according to methods described herein to determine an expression level of each of two of more gene products of a plurality of genes described herein. In certain aspects, a kit can comprise a panel of primers that can detect an expression level of at least 68 gene products of a plurality of genes described herein (for example, the panel described in section (1) above.

Primers as described herein can further be conjugated to a fluorescent label. Suitable fluorescent labels include, for example and without limitation, fluorescein, fluorescein isothiocyanate (FITC), green fluorescent protein (GFP), DyLight™ 488, phycoerythrin (PE), propidium iodide (PI), PerCP, PE-Alexa Fluor® 700, Cy5, allophycocyanin, and Cy7. Such labels can emit light of a given wavelength that can be detected by an optical detection device as described herein.

The kit can be a package which houses a container which contains compounds of the disclosure or formulations of the disclosure and also houses instructions for administering the compounds or formulations to a subject. The disclosure further relates to a commercial package comprising compounds of the disclosure or formulations of the disclosure together with instructions for simultaneous, separate or sequential use. In particular a label may include amount, frequency, and method of administration.

The disclosure also provides a pharmaceutical pack or kit comprising one or more containers filled with one or more therapeutics to provide a therapeutic effect. Associated with such container(s) can be various written materials such as instructions for use, or a notice in the form prescribed by a governmental agency regulating the labeling, manufacture, use or sale of pharmaceuticals or biological products, which notice reflects approval by the agency of manufacture, use, or sale for human administration.

The disclosure also relates to articles of manufacture and kits containing materials useful for treating a disease disclosed herein. An article of manufacture may comprise a container with a label. Examples of suitable containers include bottles, vials, and test tubes, or a delivery device such as a nebulizer, which may be formed from a variety of materials including glass and plastic. A container holds compounds of the disclosure or formulations of the disclosure which are effective for treating a disease disclosed herein. The label on the container indicates that the compounds of the disclosure or formulations of the disclosure are used for treating a disease disclosed herein and may also indicate directions for use. In aspects of the disclosure, a medicament or formulation in a container may comprise any of the medicaments or formulations disclosed herein.

The disclosure also contemplates kits comprising one or more of compounds of the disclosure. In aspects of the disclosure, a kit of the disclosure comprises a container described herein. In particular aspects, a kit of the disclosure comprises a container described herein and a second container comprising a buffer. A kit may additionally include other materials desirable from a commercial and user standpoint, including, without limitation, buffers, diluents, filters, needles, syringes, and package inserts with instructions for performing any methods disclosed herein (e.g., methods for treating a disease disclosed herein). A medicament or formulation in a kit of the disclosure may comprise any of the formulations or compositions disclosed herein.

The compositions (i.e., those comprising, consisting essentially of, or consisting of primers described herein or known in the art) can be utilized in the preparation of a kit. In some embodiments, kits are provided for carrying out any of the methods described herein. The kits of this disclosure may comprise a carrier container being compartmentalized to receive in close confinement one or more containers such as vials, tubes, and the like, each of the containers comprising one of the separate elements to be used in the methods.

In some instances, one of the containers may comprise a composition as described in this disclosure that is, or can be, detectably labeled. The kit may also have containers containing buffer(s) and/or a container comprising a reporter-means, such as a biotin-binding protein, such as avidin or streptavidin, bound to a reporter molecule, such as an enzymatic or fluorescent label. In some embodiments, the kit comprises separate containers containing compositions described herein and a detectable label.

In various embodiments of the present disclosure, a kit for determining a tumor status in one or more samples from a subject can include a plurality of primer pairs for determining expression levels of one or more of a plurality of genes related to ribosomal biogenesis, and instructions for use.

### III. Methods of Use and Treatment

As described herein, the present disclosure provides a method of determining a tumor status, particularly a tumor exhibiting altered ribosomal biogenesis compared to normotypical healthy individuals (i.e., a subject “in need thereof”). The methods described herein, after determining a tumor status, can further comprise administering to the subject a therapeutically effective amount of one or more compositions (i.e., those comprising, consisting essentially of, or consisting of therapeutics described herein) according to the present disclosure. In examples, the subject can have or can be suspected of having one or more of hepatocellular carcinoma (LIHC), kidney renal clear cell carcinoma (KIRC), a breast cancer, lung adenocarcinoma (LUAD), lung squamous cell carcinoma (LUSC), non-small cell lung cancer (NSCLC), melanoma, urothelial cancer, bladder cancer, glioblastoma (GBM), colorectal cancer, ovarian cancer, stomach adenocarcinoma, or endometrial cancer. In some embodiments, the subject has or is suspected of having hepatocellular carcinoma (LIHC), kidney renal clear cell carcinoma (KIRC), a breast cancer, or lung adenocarcinoma (LUAD). In examples, methods can first comprise obtaining a biological sample from a patient or subject, for example, a tumor biopsy from a patient having or suspected of having a cancer as described herein (i.e., hepatocellular carcinoma (LIHC), kidney renal clear cell carcinoma (KIRC), a breast cancer, or lung adenocarcinoma (LUAD)).

In In embodiments, a treatment for hepatocellular carcinoma (LIHC) can comprise surgical resection, liver transplantation, transarterial chemoembolization (TACE), radiofrequency ablation (RFA), systemic targeted therapies (e.g., sorafenib, lenvatinib, or regorafenib), immune checkpoint inhibitors (e.g., atezolizumab plus bevacizumab, nivolumab, or pembrolizumab), or a combination thereof. In embodiments, a ribosomal biogenesis score can help determine or otherwise inform a treatment status of a subject by stratifying patients into high versus low score groups, wherein a high ribosomal biogenesis score indicates a more aggressive tumor phenotype with an immunosuppressive microenvironment, characterized by reduced CD8+ T cell infiltration and increased M2 macrophage polarization, thereby informing a clinical decision to escalate systemic therapy, prioritize immune checkpoint inhibitor-based regimens, or enroll the subject in clinical trials targeting ribosomal biogenesis pathways; conversely, a low score may support standard first-line therapy or less intensive treatment approaches.

In embodiments, a treatment for kidney renal clear cell carcinoma (KIRC) can comprise surgical nephrectomy (partial or radical), targeted therapies (e.g., sunitinib, pazopanib, axitinib, cabozantinib, or everolimus), immune checkpoint inhibitors (e.g., nivolumab, pembrolizumab, or ipilimumab), combination regimens (e.g., nivolumab plus ipilimumab or pembrolizumab plus axitinib), or a combination thereof. In embodiments, a ribosomal biogenesis score can help determine or otherwise inform a treatment status of a subject by identifying high-score subjects whose tumors exhibit diminished CD8+ T cell and NK cell infiltration alongside enrichment of suppressive myeloid populations (M2 macrophages, neutrophils, and MDSC-like cells) and pronounced stromal expansion, thereby informing a clinical decision to prioritize combination immune checkpoint inhibitor regimens or therapies targeting the immunosuppressive microenvironment; additionally, a high score correlating with elevated aneuploidy and proliferation indices may further inform decisions regarding more aggressive systemic therapy selection or clinical trial enrollment.

In embodiments, a treatment for a breast cancer can comprise surgical intervention (lumpectomy or mastectomy), radiation therapy, chemotherapy (e.g., anthracycline- or taxane-based regimens), endocrine therapies (e.g., tamoxifen or aromatase inhibitors) for hormone receptor-positive disease, HER2-targeted therapies (e.g., trastuzumab, pertuzumab, or T-DM1) for HER2-positive disease, CDK4/6 inhibitors (e.g., palbociclib or ribociclib), PARP inhibitors (e.g., olaparib or talazoparib) for BRCA-mutated disease, immune checkpoint inhibitors (e.g., pembrolizumab or atezolizumab) particularly for triple-negative breast cancer (TNBC), or a combination thereof. In embodiments, a ribosomal biogenesis score can help determine or otherwise inform a treatment status of a subject by identifying high-score subjects, particularly those with basal-like or HER2-positive subtypes and higher Nottingham histological grades, who face significantly worse overall, relapse-free, and disease-specific survival outcomes, thereby informing decisions to intensify systemic therapy, prioritize immune checkpoint inhibitor-based regimens, or monitor more closely for early relapse; furthermore, because a high score correlates with elevated cancer stemness indices, it may additionally inform decisions to incorporate therapies targeting stem cell-like properties or resistance mechanisms.

In embodiments, a treatment for lung adenocarcinoma (LUAD) can comprise surgical resection, radiation therapy, platinum-based chemotherapy doublets (e.g., carboplatin or cisplatin with pemetrexed or paclitaxel), targeted therapies directed against oncogenic driver mutations (e.g., EGFR inhibitors such as osimertinib, ALK inhibitors such as alectinib, or KRAS G12C inhibitors such as sotorasib), immune checkpoint inhibitors (e.g., pembrolizumab, nivolumab, or atezolizumab), or a combination thereof. In embodiments, a ribosomal biogenesis score can help determine or otherwise inform a treatment status of a subject by stratifying high-score LUAD patients, whose tumors show a marked effector immune cell deficit alongside dominant M2 macrophage and neutrophil polarization, toward immune checkpoint inhibitor-based or combination regimens targeting both ribosomal biogenesis and immune checkpoints; additionally, a high score correlating with elevated aneuploidy and proliferation indices may further support intensification of systemic chemotherapy or enrollment in clinical trials targeting RNA polymerase I or other upstream regulators of ribosomal RNA transcription, whereas a low score may support standard first-line or driver mutation-based targeted therapy alone.

In any of the above embodiments, a ribosomal biogenesis score above a defined threshold, for example, above the median or in the top quartile of a reference population, can indicate a subject who may benefit from more aggressive therapeutic intervention, immune checkpoint inhibitor-based therapy, or clinical trial enrollment targeting ribosomal biogenesis pathways, while a score below said threshold may indicate a subject suitable for standard-of-care therapy or less intensive treatment regimens.

In examples, a ribosomal biogenesis score (RB) described herein can be useful in assessing the usefulness of one or more therapies, including but not limited to the following as described below: immune checkpoint inhibitors; RNA Pol I inhibitors; mTor inhibitors; conventional chemotherapy and anti-proliferative agents; and therapies targeting cancer stemness.

In examples, a RB score described herein can be useful in assessing the usefulness of immune checkpoint inhibitor therapies, including anti-PD-1 therapies (e.g., nivolumab or pembrolizumab), anti-PD-L1 therapies (e.g., atezolizumab or durvalumab), and anti-CTLA-4 therapies (e.g., ipilimumab), wherein elevated RB scores may associate with improved therapeutic benefit and better overall survival outcomes in patients receiving said immune checkpoint inhibitor therapies, as demonstrated across multiple cancer types including non-small cell lung cancer, melanoma, urothelial cancer, glioblastoma, and kidney clear cell carcinoma (HR ranging from 0.42 to 0.74; log-rank p<0.05 across all cohorts); accordingly, a high RB score may identify subjects who are more likely to benefit from immune checkpoint inhibitor-based therapies, thereby informing patient selection and treatment decisions.

In examples, a RB score described herein can be useful in assessing the usefulness of RNA Polymerase I (Pol I) inhibitors (e.g., CX-5461), which directly target ribosomal RNA transcription upstream of ribosomal biogenesis, wherein a high RB score may identify subjects who are more likely to benefit from targeted inhibition of ribosomal biogenesis pathways, either as monotherapy or in combination with immune checkpoint inhibitors; conversely, a low RB score may indicate subjects less likely to benefit from Pol I inhibitor-based therapies.

In examples, a RB score described herein can be useful in assessing the usefulness of mTOR inhibitors (e.g., everolimus or temsirolimus), wherein a high RB score, reflecting upregulated ribosomal biogenesis driven in part by mTOR pathway activation, may identify subjects who are more likely to benefit from mTOR-targeted therapeutic strategies, either as monotherapy or in combination with other targeted or immune-based therapies.

In examples, a RB score described herein can be useful in assessing the usefulness of conventional chemotherapy and anti-proliferative agents, including but not limited to platinum-based regimens (e.g., carboplatin or cisplatin), taxane-based regimens (e.g., paclitaxel or docetaxel), anthracycline-based regimens (e.g., doxorubicin), and antimetabolites (e.g., pemetrexed or gemcitabine), wherein a high RB score, which correlates positively with tumor proliferation indices and aneuploidy, may identify subjects with rapidly proliferating tumors that are more likely to respond to cytotoxic chemotherapy regimens, or alternatively, may identify subjects at greater risk of therapeutic resistance, thereby informing decisions to intensify, modify, or combine chemotherapy regimens with other therapeutic strategies.

In examples, a RB score described herein can be useful in assessing the usefulness of therapies targeting cancer stemness, wherein a high RB score, which correlates positively with RNA expression-based stemness indices, may identify subjects whose tumors harbor pronounced stem-like properties, informing decisions to incorporate therapies targeting stem cell renewal pathways or resistance mechanisms in combination with standard-of-care treatments.

In any of the above embodiments, a RB score above a defined threshold, for example, above the median or in the top quartile of a reference population, can indicate a subject who may benefit from immune checkpoint inhibitor-based therapy, Pol I inhibition, mTOR-targeted therapy, or intensified chemotherapy regimens, while a RB score below said threshold may indicate a subject suitable for standard-of-care therapy or less intensive treatment regimen.

In examples, a RB score described herein may further be useful in the context of T cell-based therapies, including chimeric antigen receptor T cell (CAR-T) therapy, tumor-infiltrating lymphocyte (TIL) therapy, and T cell receptor-engineered T cell (TCR-T) therapy, in two potential applications:

In examples, a RB score determined from a tumor sample of a subject may inform the likely response of said subject to T cell-based therapies, wherein a high tumor RB score, reflecting an immunosuppressive tumor microenvironment characterized by reduced CD8+ T cell infiltration and enrichment of suppressive myeloid populations, may identify subjects whose tumors present significant barriers to T cell infiltration, persistence, and function, thereby informing clinical decisions regarding patient selection and combination therapeutic strategies for T cell-based therapies.

In examples, a RB score determined from a T cell product derived from a subject prior to infusion in the context of adoptive T cell therapy may further reflect the functional fitness, stemness, and durability of said T cell product, wherein higher ribosomal biogenesis activity in T cells may identify T cell products enriched for stem-like memory phenotypes with superior proliferative capacity and persistence following infusion, thereby informing decisions to optimize the T cell manufacturing process or T cell product prior to patient infusion

Various embodiments of the present disclosure provide a method of identifying a tumor status, including detecting an expression level of one or more of a plurality of genes related to ribosomal biogenesis in one or more samples from a subject, assigning one or more values to the expression level of individual ones of the plurality of genes, summing the one or more assigned values of individual ones of the plurality of genes, comparing a summed value to a threshold, and assigning the tumor status of the subject based at least in part on a comparison of the summed value to the threshold. In some examples, the tumor status can be an immune infiltration status, a stemness status, a reduced overall survival, or a reduced progression-free interval. In various examples, the subject can have or can be suspected of having one or more of hepatocellular carcinoma (LIHC), kidney renal clear cell carcinoma (KIRC), a breast cancer, lung adenocarcinoma (LUAD), lung squamous cell carcinoma (LUSC), non-small cell lung cancer (NSCLC), melanoma, urothelial cancer, bladder cancer, glioblastoma (GBM), colorectal cancer, ovarian cancer, stomach adenocarcinoma, or endometrial cancer. In various examples, the subject can have or can be suspected of having hepatocellular carcinoma (LIHC), kidney renal clear cell carcinoma (KIRC), a breast cancer, or lung adenocarcinoma (LUAD). In some examples, the method can further include outputting a report comprising the one or more assigned values, the summed value, the assigned tumor status, or a combination of any thereof. In some examples, the subject can be administered a therapy based at least in part on the tumor status assigned to the subject. In further examples, the threshold can be a median of the summed value of a plurality of subjects or a quartile of the summed value of the plurality of subjects.

The compositions described herein are useful in, inter alia, methods for treating a cancer in a subject. In examples, the subject can have or can be suspected of having one or more of hepatocellular carcinoma (LIHC), kidney renal clear cell carcinoma (KIRC), a breast cancer, lung adenocarcinoma (LUAD), lung squamous cell carcinoma (LUSC), non-small cell lung cancer (NSCLC), melanoma, urothelial cancer, bladder cancer, glioblastoma (GBM), colorectal cancer, ovarian cancer, stomach adenocarcinoma, or endometrial cancer. In some embodiments, the subject has or is suspected to have hepatocellular carcinoma (LIHC), kidney renal clear cell carcinoma (KIRC), a breast cancer, or lung adenocarcinoma (LUAD). In some embodiments, the subject is diagnosed with a cancer. In some embodiments, the subject is a human that is suspected of having a cancer.

The compositions can be administered to a subject, e.g., a human subject, using a variety of methods that depend, in part, on the route of administration. The route can be, e.g., intravenous injection or infusion (IV), subcutaneous injection (SC), intraperitoneal (IP) injection, intramuscular injection (IM), intradermal injection (ID), subcutaneous, transdermal, intracavity, oral, or intrathecal injection (IT). The injection can be in a bolus or a continuous infusion. Techniques for preparing injectate or infusate delivery systems containing antibodies are well known to those of skill in the art. Generally, such systems should utilize components which will not significantly impair the biological properties of the antibodies, such as the paratope binding capacity (see, for example, Remington's Pharmaceutical Sciences, 18th edition, 1990, Mack Publishing). Those of skill in the art can readily determine the various parameters and conditions for producing antibody injectates or infusates without resort to undue experimentation. In some embodiments, compositions as described herein can be therapeutically delivered to a subject by way of colonic or oral administration.

Treating or treatment of any disease or disorder refers to ameliorating a disease or disorder that exists in a subject or a symptom thereof, in particular, ameliorating symptoms of a cancer. The term ameliorating refers to any therapeutically beneficial result in the treatment of a disease state, e.g., improved survival.

Thus, treating or treatment includes ameliorating at least one parameter or symptom. Treating or treatment includes modulating the disease or disorder, either physically (e.g., stabilization of a discernible symptom) or physiologically (e.g., stabilization of a physical parameter) or both. Thus, in the disclosed methods, treatment can refer to a 10%, 20%, 30%, 40%, 50%, 60%, 70%, 80%, 90%, or 100% reduction in the severity of an established disease or condition or symptom of the disease or condition. For example, a method for treating a cancer in a subject by administering a composition as described in this disclosure is considered to be a treatment or therapeutic, for example, if there is a 10% improvement according to the measured or observed parameter in a subject as compared to a control. Thus, the reduction can be a 10%, 20%, 30%, 40%, 50%, 60%, 70%, 80%, 90%, 100%, or more (or any percent improvement in between 10% and 100%) as compared to native or control levels. It is understood that treatment does not necessarily refer to a cure or complete ablation of the disease, condition, or symptoms of the disease or condition.

Described herein are therapeutically effective amounts as defined previously. A therapeutically effective amount is not, however, a dosage so large as to cause adverse side effects. A suitable dose capable of ameliorating a cancer in a subject, can depend on a variety of factors including the particular construct used and whether it is used concomitantly with other therapeutic agents. Generally, a therapeutically effective amount may vary with the subject's age, condition, and sex, as well as the extent of the disease in the subject and can be determined by one of skill in the art. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject. It should also be understood that a specific dosage and treatment regimen for any particular subject also depends upon the judgment of the treating medical practitioner (e.g., doctor or nurse). A therapeutically effective amount is also one in which any toxic or detrimental effects of the composition are outweighed by the therapeutically beneficial effects. The dosage of the therapeutically effective amount may be adjusted by the individual physician or veterinarian in the event of any complication.

A pharmaceutical composition can include a therapeutically effective amount of a therapeutic described herein. Such effective amounts can be readily determined by one of ordinary skill in the art as described above.

Suitable human doses of any of the therapeutics described herein can further be evaluated in, e.g., Phase I dose escalation studies. See, e.g., van Gurp et al. (2008) Am J Transplantation 8(8): 1711-1718; Hanouska et al. (2007) Clin Cancer Res 13(2, part 1):523-531; and Hetherington et al. (2006) Antimicrobial Agents and Chemotherapy 50 (10): 3499-3500.

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

Other features, objects, and advantages of the present invention are apparent in the description that follows. It should be understood, however, that the description, while exemplifying certain embodiments of the present invention, is given by way of illustration only, not limitation. Various changes and modifications within the scope of the invention will become apparent to those skilled in the art from the detailed description.

### IV. Examples

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is in atmosphere. Standard temperature and pressure are defined as 25° C. and 1 atmosphere.

### Example 1

### Introduction

Cancer progression is fundamentally driven by a complex interplay between oncogenic signaling, cellular metabolism, and the tumor microenvironment. Among these processes, ribosome biogenesis (RB)—the synthesis and assembly of ribosomal components—has emerged as an often underappreciated contributor to tumor development. Rapidly proliferating cancer cells frequently upregulate RB to meet their heightened demand for protein synthesis, and this increased activity can be linked to aggressive phenotypes, therapeutic resistance, and poor clinical outcomes. Despite growing interest in targeting RB therapeutically, the precise role and prognostic value of RB-related genes across different cancer types remains an active area of investigation.

Recent work has shed light on how altered ribosomal protein abundance or rRNA processing may serve as biomarkers and drivers of malignancy. For instance, some studies suggest that disruptions in the nucleolar architecture—where RB takes place—can activate oncogenic pathways, including MYC, or interfere with tumor suppressors, such as p53. However, the underlying mechanisms by which elevated RB informs worse survival or increased metastatic potential are still debated. Conflicting findings from various experimental models highlight the need for a robust, integrative approach that can capture the collective contribution of multiple RB-related genes rather than focusing on a single component.

Here, an RB score derived from a curated set of 541 RB-associated genes is proposed that captures overall RB activity across multiple cancer types. By applying feature selection (LASSO) and carefully assigning directionality (i.e., whether higher expression of a gene predicts better or worse outcomes), this large gene set was distilled into a streamlined, prognostically relevant signature. The RB score was evaluated in major clinical datasets (including GSE202203 and TCGA), testing its association with diverse outcomes such as overall survival, progression-free interval, and immunotherapy response. The findings suggest that heightened RB activity, as reflected by the composite score, correlates with tumor aggressiveness, immune evasion, and diminished survival. It was further explored how integrating this RB score with standard immune-oncology biomarkers might refine patient stratification and guide treatment decisions. Thus, various embodiments of the present disclosure aim to establish a robust framework for quantifying RB activity in solid tumors and to underscore its prognostic and potential therapeutic value.

### Materials and Methods

### Data Collection and Preprocessing

541 genes associated with RB were identified via comprehensive literature and database searches (PubMed, Gene Ontology, GeneCards, UniProt, Online Mendelian Inheritance in Man (OMIM), and PANTHER) and used as the candidate RB gene set. Then, normalized gene expression and clinical data were retrieved from multiple sources:
GSE202203 (training cohort): Expression data restricted to the 541 RB genes were acquired from the Gene Expression Omnibus (GEO).
The Cancer Genome Atlas (TCGA): Processed gene expression and matched clinical outcomes for pan-cancer analyses were accessed via the University of California, Santa Cruz Xena Browser.

All datasets were checked for completeness, and missing expression or clinical values were omitted or set to zero for consistent downstream analysis. Genes or samples with insufficient data were excluded from the final analysis.

### RB Score Construction Via LASSO Regression

To narrow down the 541-gene set, a LASSO (Least Absolute Shrinkage and Selection Operator) regression model was trained on the GSE202203 cohort, focusing on overall survival (OS) as the outcome variable. LASSO was implemented in Python (version 3.10; scikit-learn library version 1.0) using 5-fold cross-validation (LassoCV). Genes with non-zero coefficients were retained. The sign of each retained coefficient determined whether higher expression predicted poorer survival (“negative” predictor) or better survival (“positive” predictor). A forward selection approach was then applied to identify an optimal subset of these LASSO-selected genes, maximizing the coefficient of determination (R\hat{ }2).

For each selected gene, a score of +1+1 or −1−1 was assigned in each sample based on whether its expression exceeded (or did not exceed) the median expression level, with the sign of the contribution dependent on the directionality inferred from the LASSO coefficient. The RB score for each sample was computed as the sum of these+1+1 values across all selected genes.

### Survival Analyses

Kaplan-Meier (KM) analyses were conducted to evaluate the prognostic impact of the RB score on multiple survival endpoints (e.g., overall survival, disease-specific survival, disease-free interval, and progression-free interval). For most cohorts, patients were stratified into two groups (e.g., High vs. Low RB score) by either:
Median cut-off: Patients above the median were assigned to the High group, and those below the median to the Low group.
Quartile-based cut-offs: Patients in the top quartile vs. bottom quartile of the RB score distribution.

Survival curves were generated in GraphPad Prism (version 10) or, where noted, in Python using the lifelines package (version 0.30.0). The log-rank test was used to compare survival distributions between groups, and hazard ratios (HR) with 95% confidence intervals were reported.

### Statistical Analyses

All statistical analyses, unless otherwise specified, were performed in GraphPad Prism. Two-sided p-values less than 0.05 were considered significant. Correlations between the RB score and continuous variables (e.g., immune infiltration scores, aneuploidy, or stemness indices) were evaluated by Pearson correlation using either GraphPad Prism or Python's pandas and numpy libraries. Where relevant, false discovery rate (FDR) corrections were applied to multiple comparisons.

### Immunotherapy Response and TIDE Analysis

The predictive value of the RB score were assessed against known immunotherapy biomarkers using the TIDE (Tumor Immune Dysfunction and Exclusion) web tool. TIDE compares user-defined biomarkers—submitted as a single or weighted gene set—to standard immunotherapy biomarkers (e.g., CD8, IFNG, TIDE, microsatellite instability (MSI) score) by generating receiver operating characteristic (ROC) curves and area under the curve (AUC) values. TIDE also provides KM plots for various checkpoint inhibitor-treated cohorts (anti-programmed cell death protein 1 (anti-PD-1), anti-programmed cell death-ligand 1 (anti-PD-L1), and anti-CTLA-4), enabling direct comparisons of survival outcomes between high and low RB score groups.

### Kaplan-Meier Plotter (Immunotherapy Module)

For further validation in immunotherapy-treated cohorts (e.g., melanoma, urothelial cancer), the KMplot immunotherapy module was employed. The list of genes of the RB score was uploaded and the genes were assigned calculated weight according to their LASSO directionality and generated survival curves stratifying patients into “High” vs. “Low” expression groups. HRs and log-rank p-values were automatically calculated by the platform, and patient subgroups were defined based on the median or quartile cut-off per the tool's default or user-defined settings.

### Immune and Stemness Analyses

Immune infiltration scores (e.g., stromal score, ESTIMATE score), as well as stemness indices (RNA expression-based stemness [RNAss]), were extracted or calculated from published pipelines (e.g., Xena Browser). Pearson's correlation was used to quantify associations between the RB score and these immune or stemness signatures, and results were visualized as scatter plots with best-fit regression lines.

### Software and Code Availability

All Python analyses, including the LASSO-based feature selection, forward selection, survival modeling (lifelines), and visualizations (matplotlib, seaborn), were performed in Python 3.10 on a Windows 10 platform. Survival curves and additional statistical comparisons were generated or refined in GraphPad Prism (version 10).

### Results

### Comprehensive Profiling of Ribosome Biogenesis Genes Reveals Broad Prognostic Value

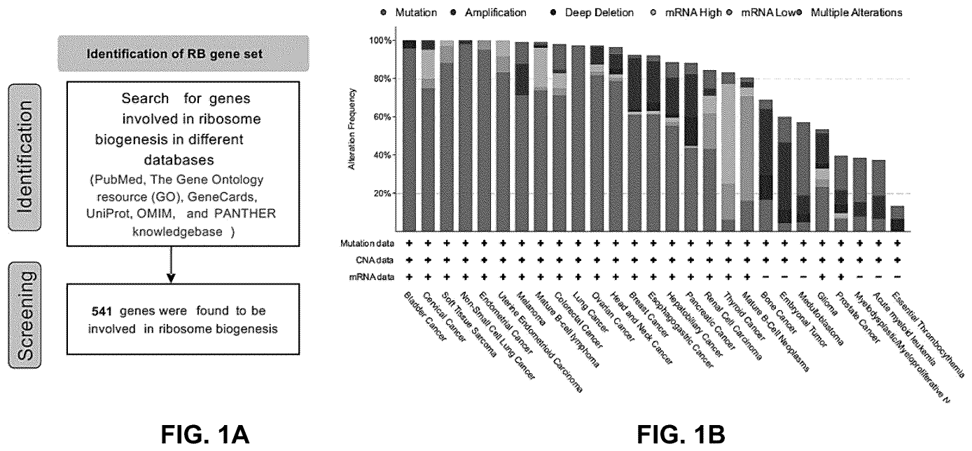

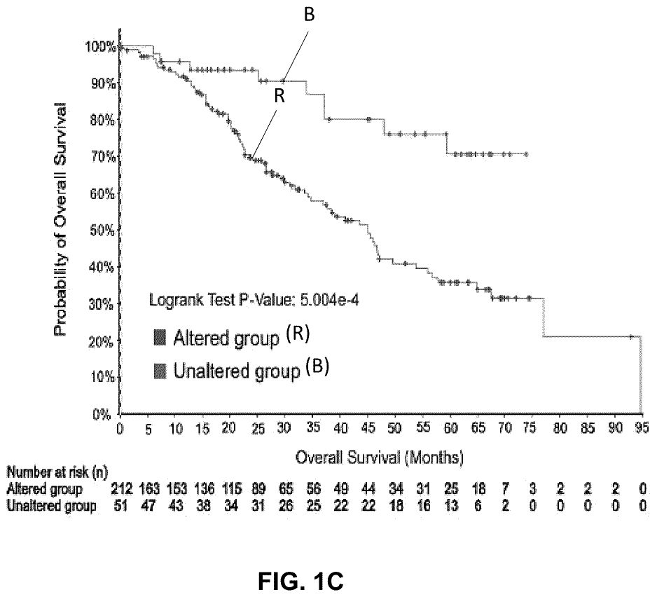

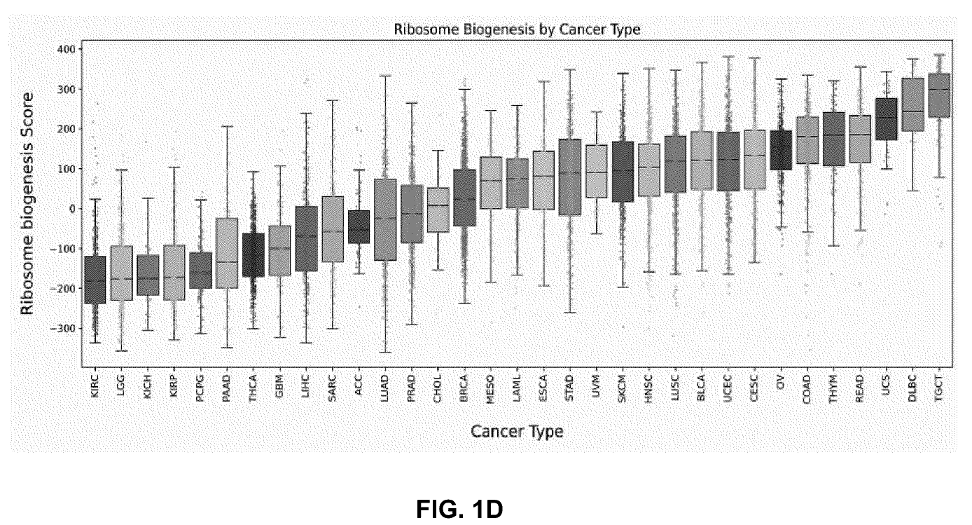

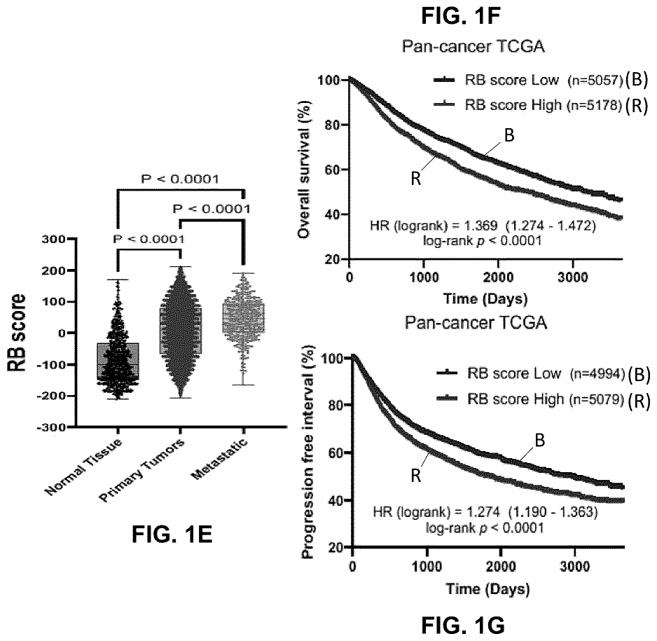

To investigate the significance of RB in cancer progression, 541 genes associated with RB were first identified through extensive database screening (FIG. 1A). These genes exhibited frequent genetic and transcriptomic alterations across diverse tumor types, including copy number amplifications, deep deletions, and significant mRNA dysregulation (FIG. 1B). Notably, patients whose tumors harbored any RB gene alteration displayed markedly worse overall survival than those without such alterations (FIG. 1C), suggesting that dysregulated ribosome biogenesis contributes to a more aggressive disease phenotype.

### RB Score Distribution and Prognostic Impact in TCGA

To quantify the collective impact of these 541 RB genes on tumor biology, an RB score was derived for each sample in The Cancer Genome Atlas (TCGA) by aggregating gene expression data. This score varied substantially across different cancer types (FIG. 1D), underscoring the heterogeneous nature of ribosome biogenesis dysregulation. Moreover, when comparing normal tissues, primary tumors, and metastatic lesions, a progressive increase in the RB score with disease stage was observed (FIG. 1E; p<0.0001), implying that heightened RB activity may be an important driver of tumor advancement.

At the pan-cancer level, TCGA samples with higher RB scores had significantly reduced overall survival (HR=1.369, log-rank p<0.0001; FIG. 1F) and shorter progression-free intervals (HR=1.274, log-rank p<0.0001; FIG. 1G). These findings strongly support the notion that an upregulated ribosome biogenesis program is broadly linked to poor clinical outcomes, regardless of tumor origin.

### Functional Enrichment of the 541 RB Genes

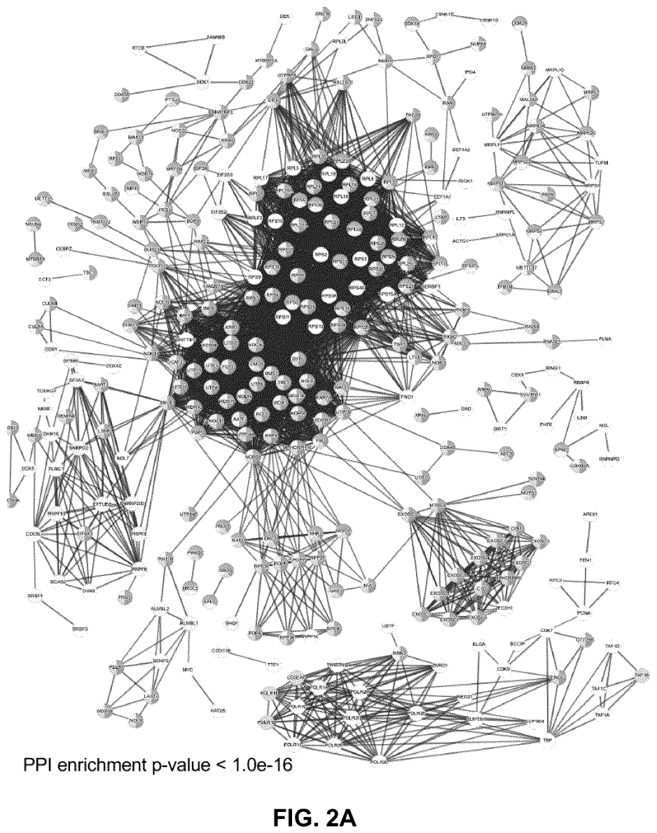

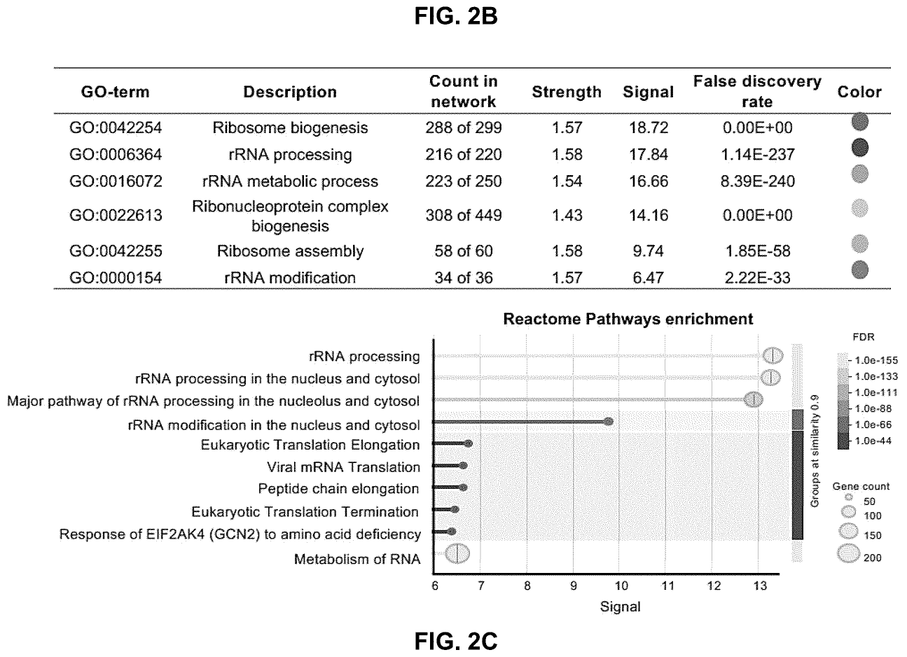

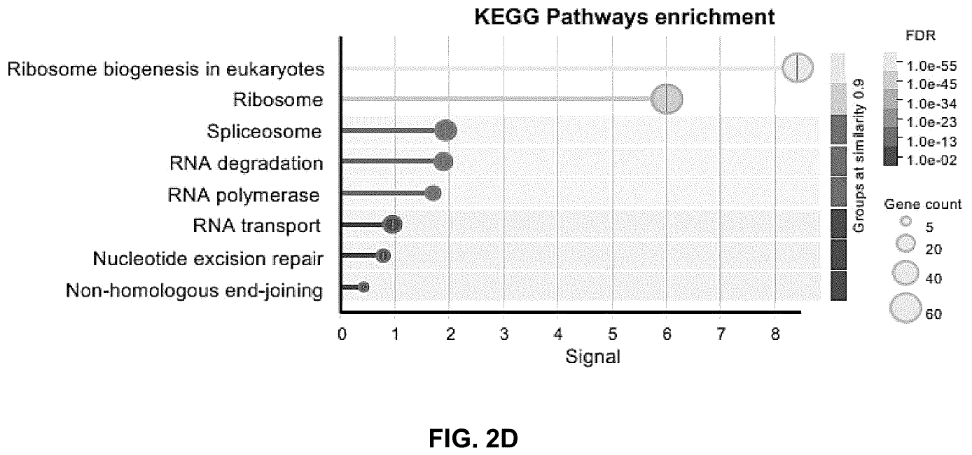

To gain deeper mechanistic insights, a protein-protein interaction (PPI) network was constructed with all 541 RB genes in STRING (FIG. 2A). This revealed dense clusters of interacting proteins, indicating extensive functional interdependencies within ribosome biogenesis pathways. Gene Ontology (GO) analysis confirmed significant enrichment for rRNA processing, ribonucleoprotein assembly, and other ribosome-related processes (FIG. 2B), while Reactome and Kyoto Encyclopedia of Genes and Genomes (KEGG) pathway analyses highlighted the role of these genes in eukaryotic translation, viral mRNA translation, RNA metabolism, and the spliceosome (FIG. 2C, 2D). Altogether, these data validate that the 541-gene RB set reflects fundamental mechanisms driving protein synthesis and suggest that dysregulation of these processes could promote tumor progression.

RB Score Stratifies Survival Endpoints across Multiple Tumor Types

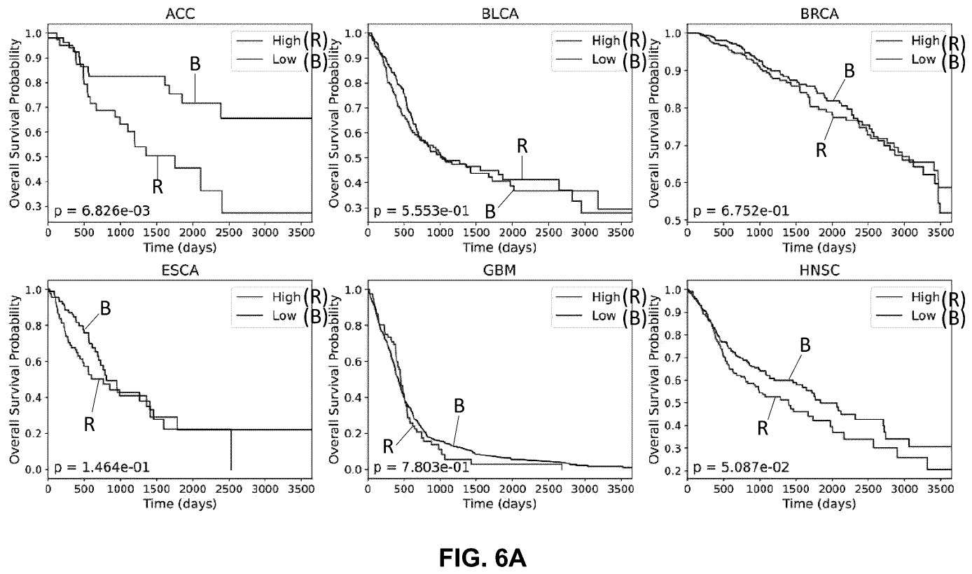

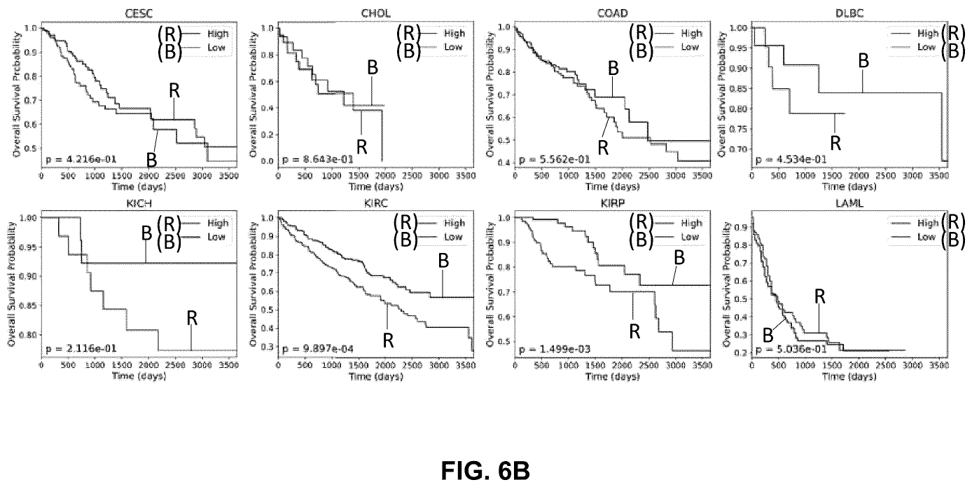

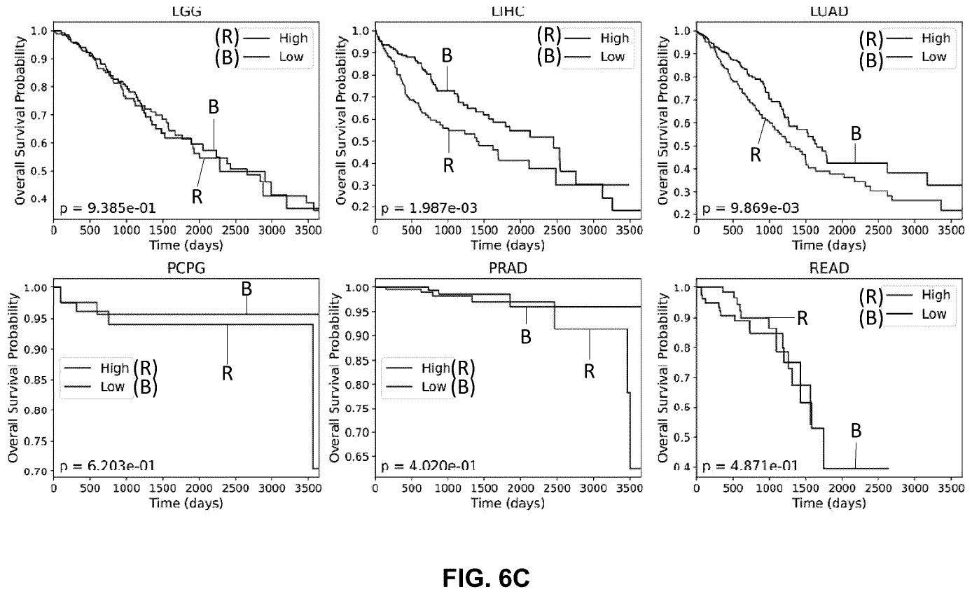

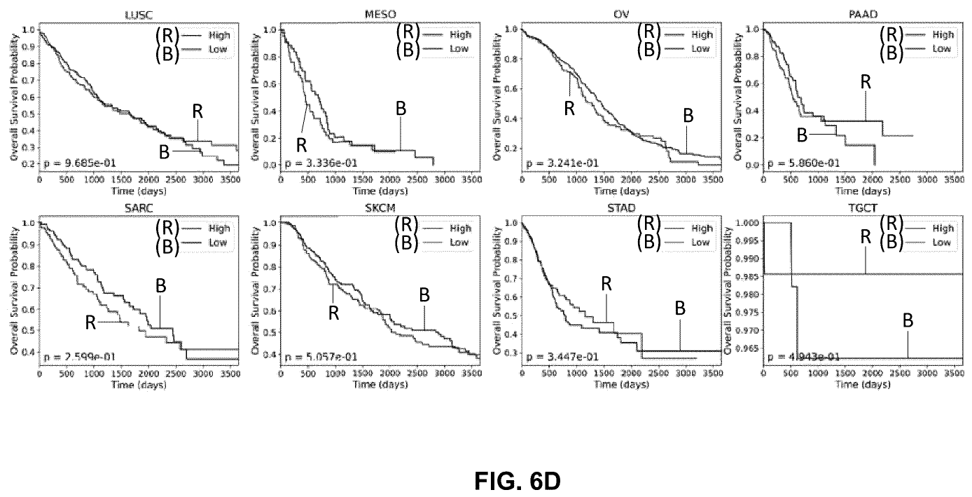

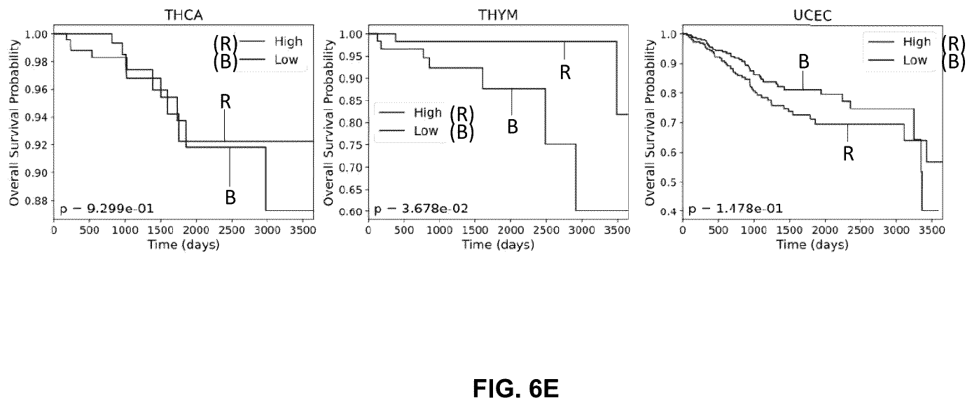

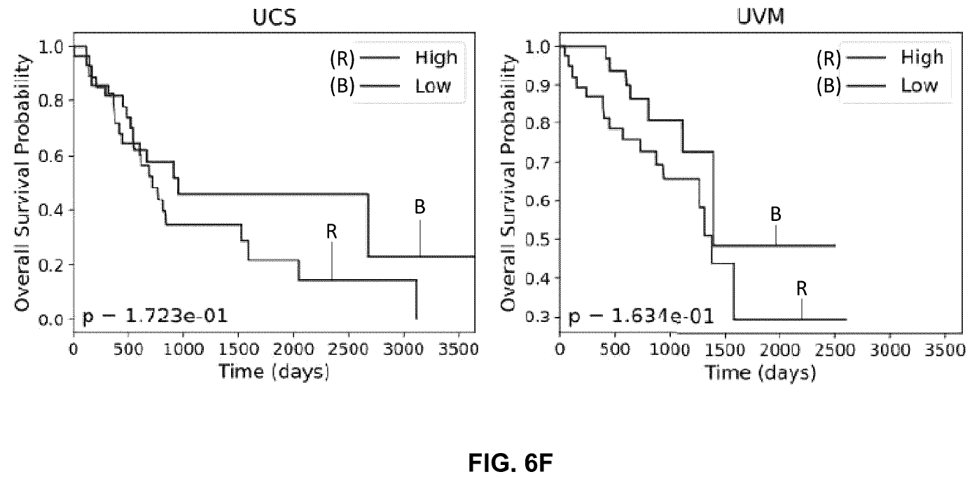

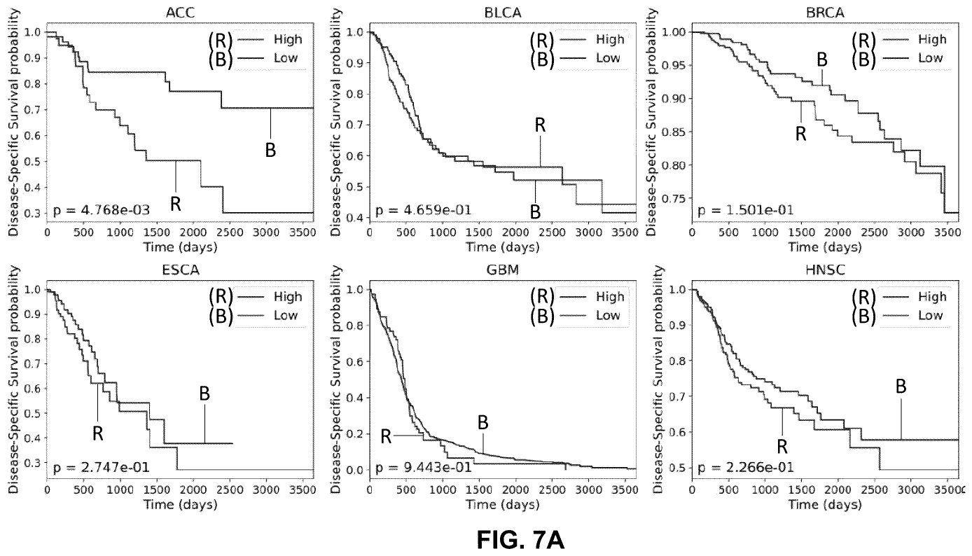

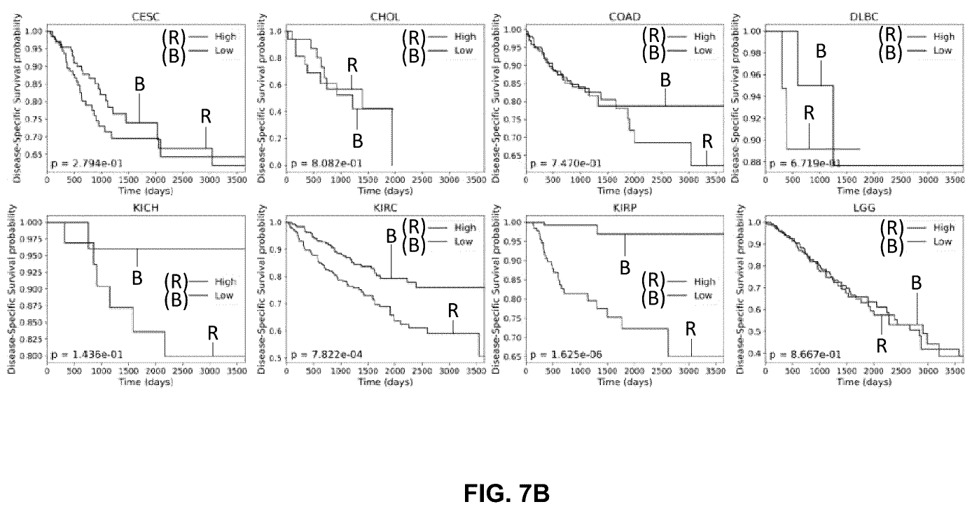

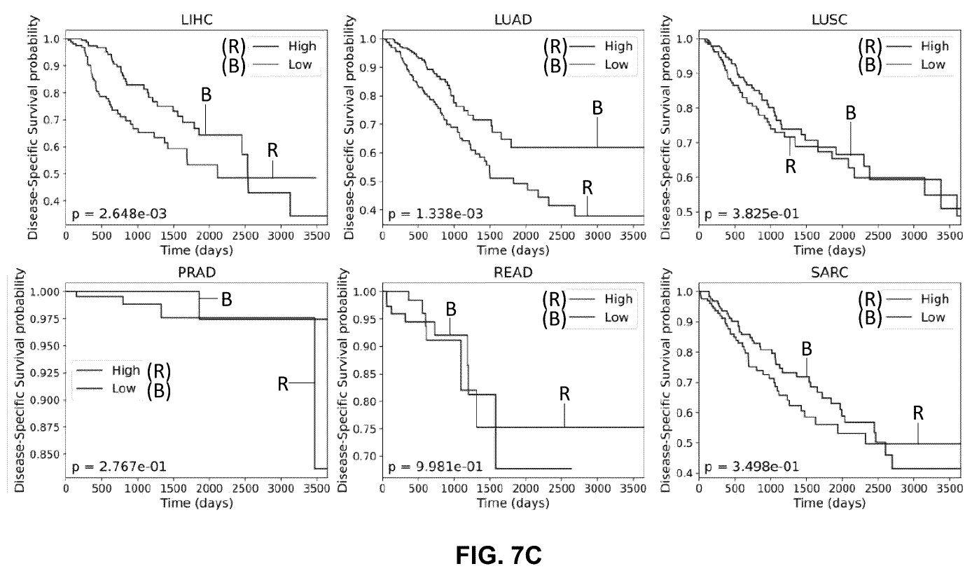

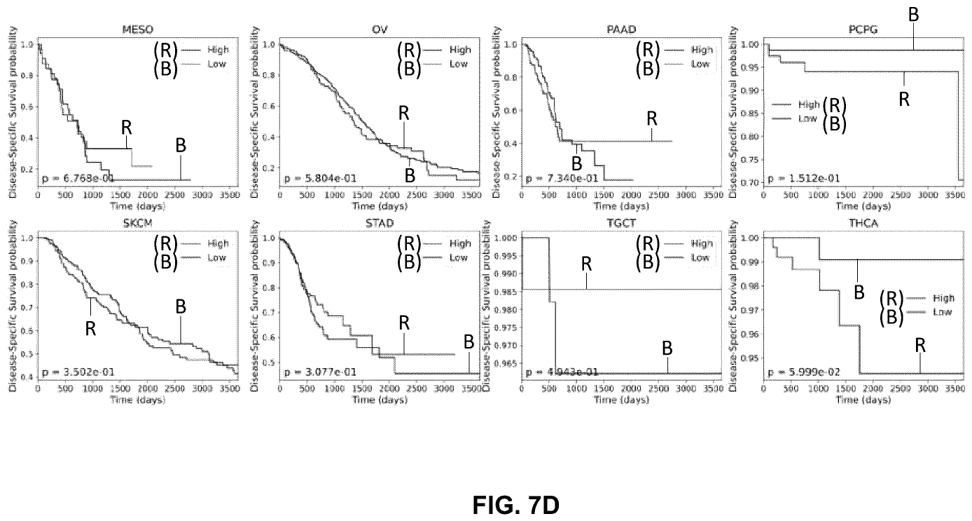

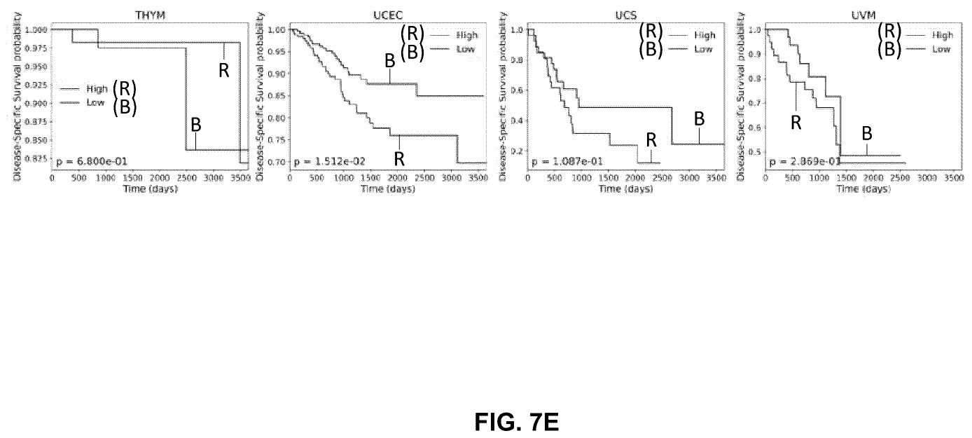

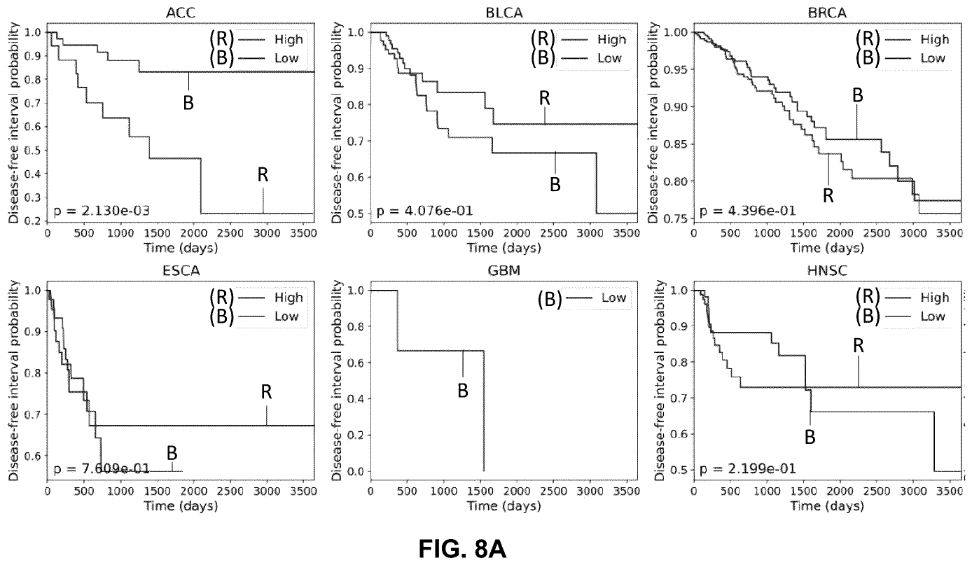

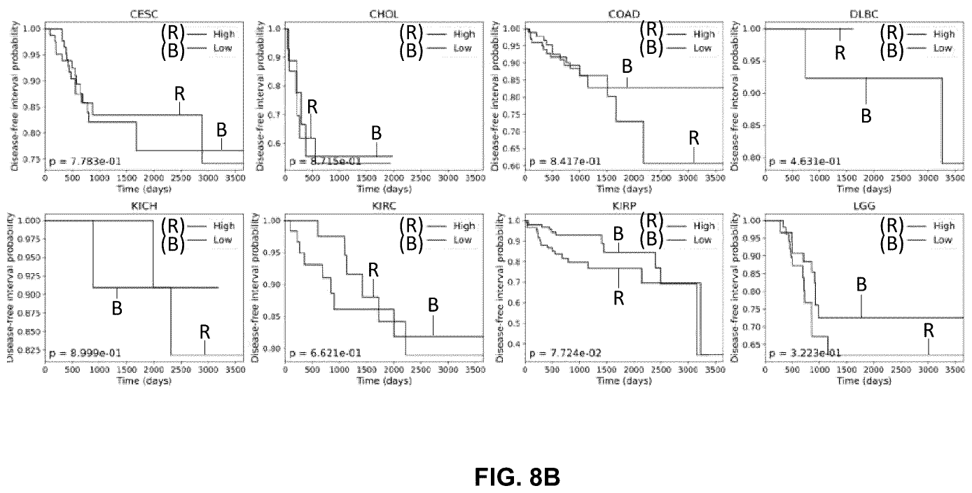

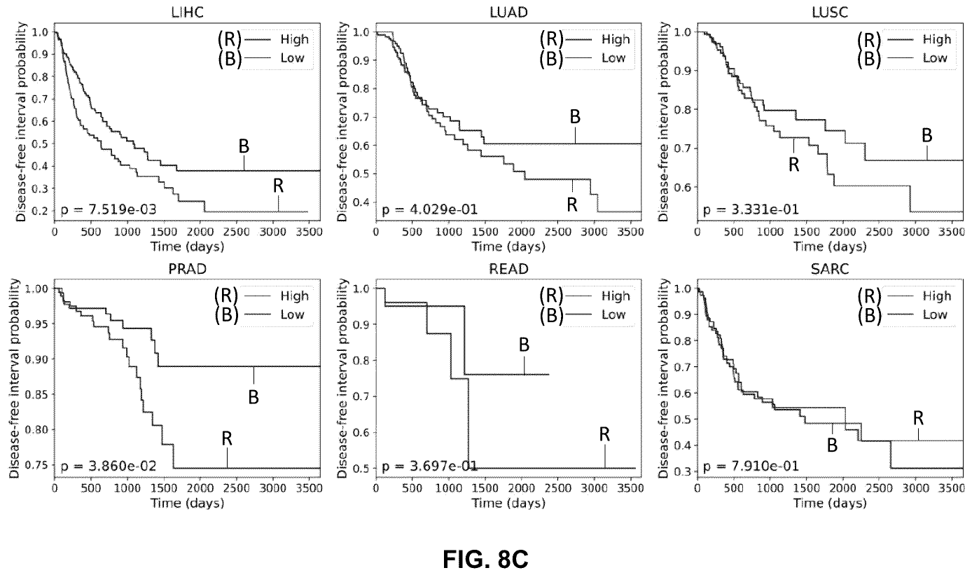

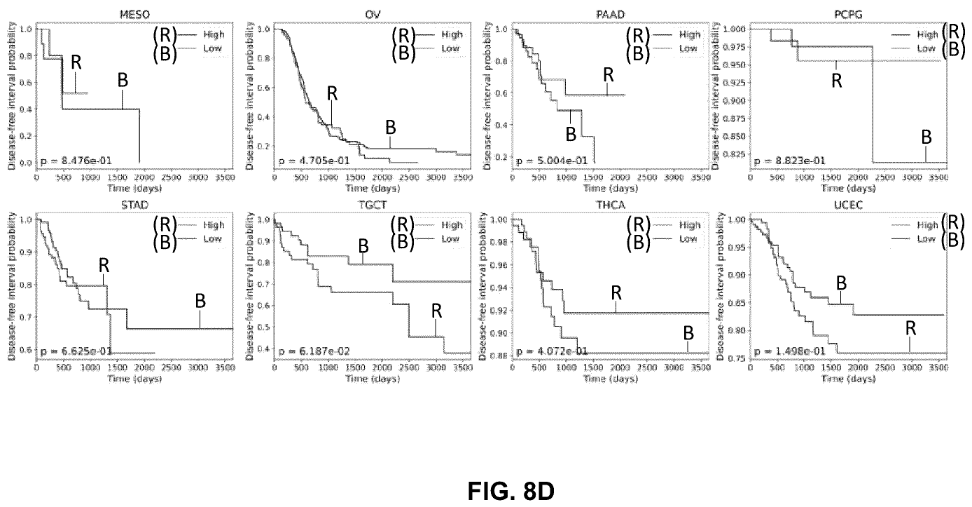

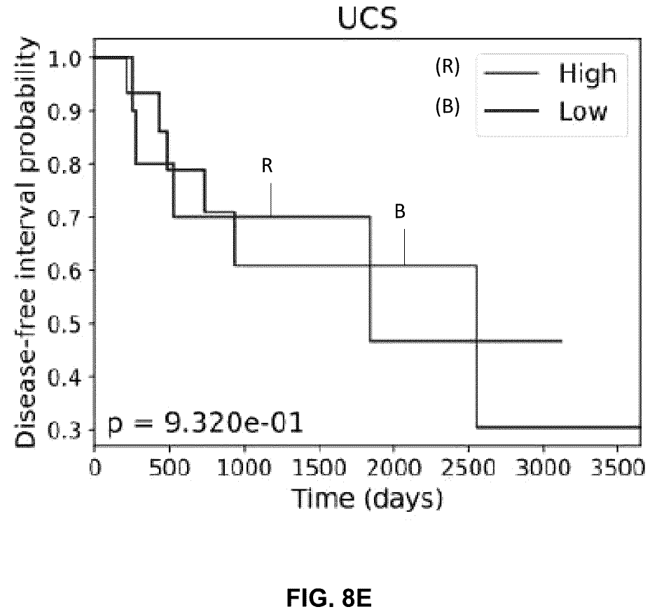

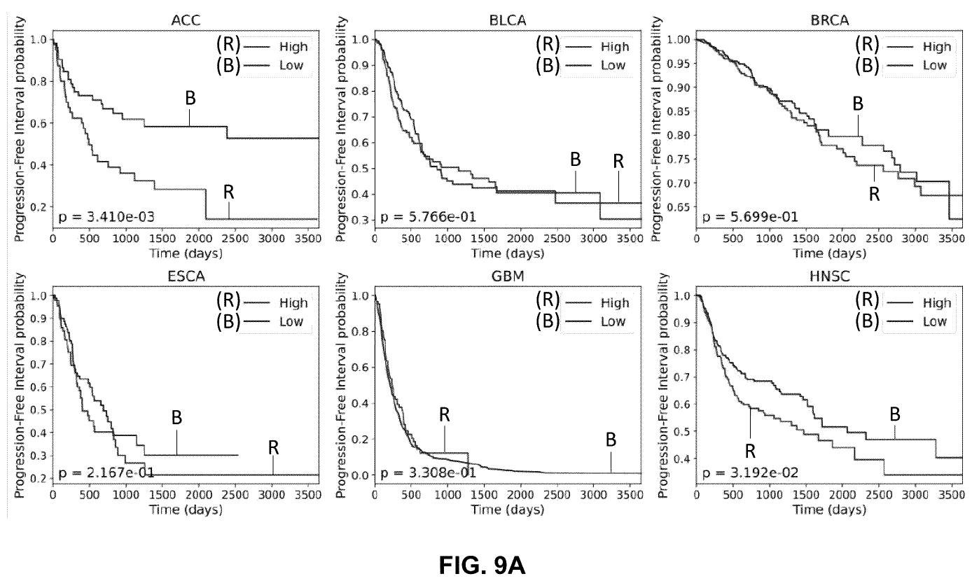

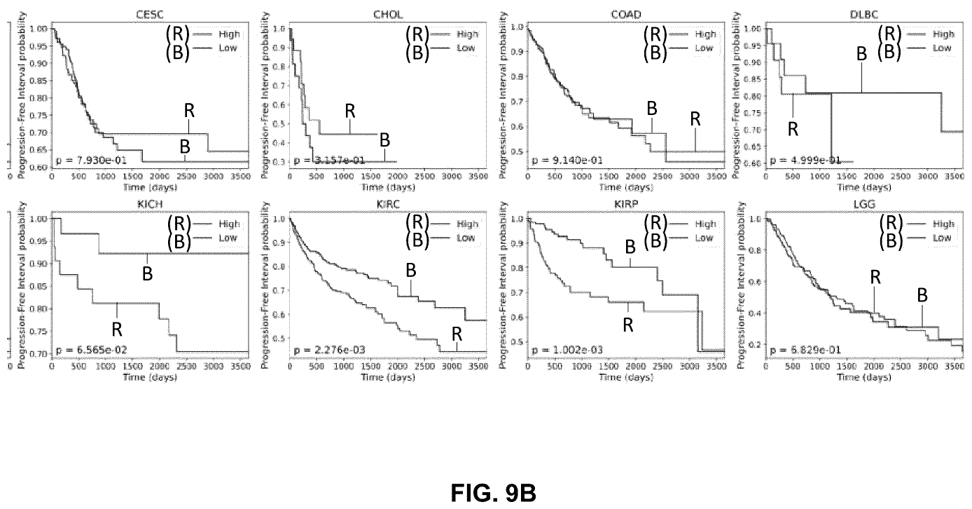

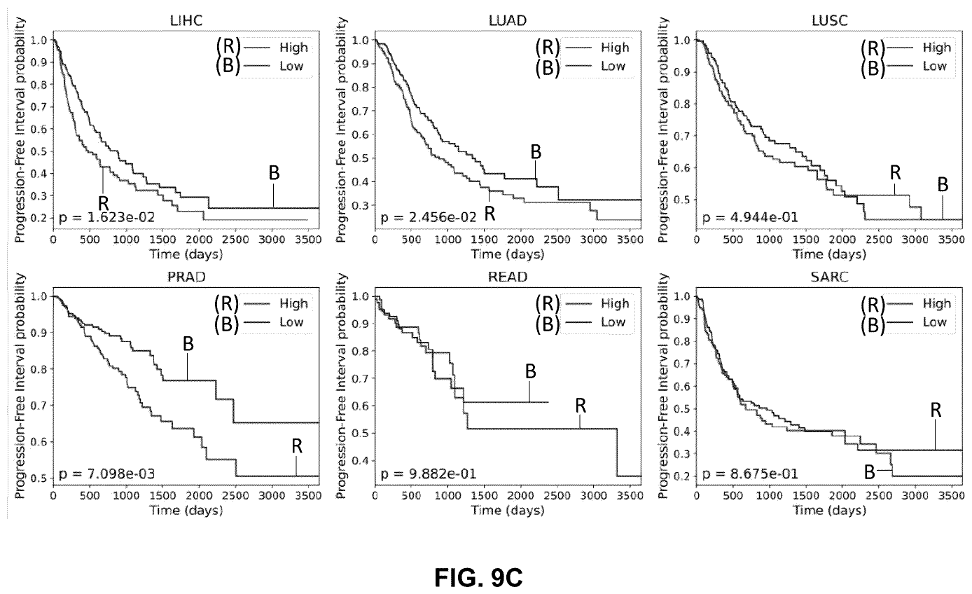

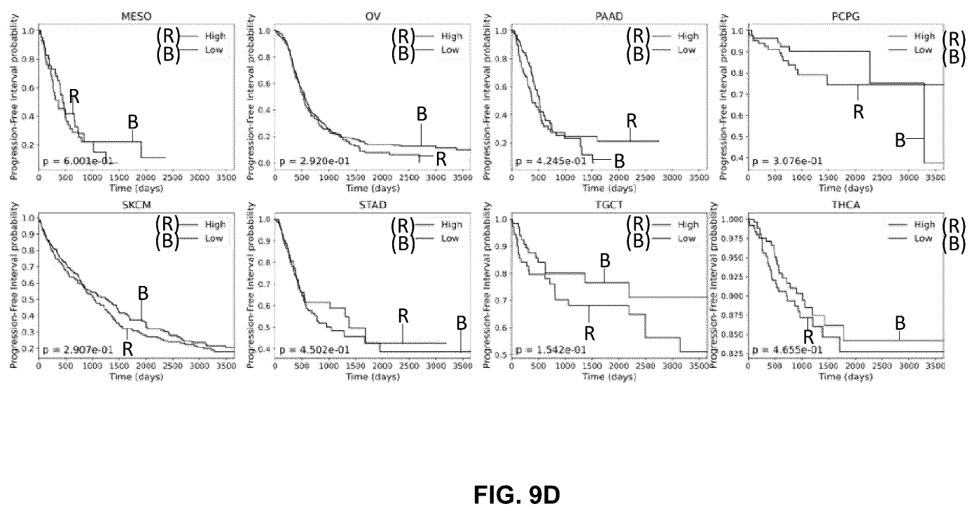

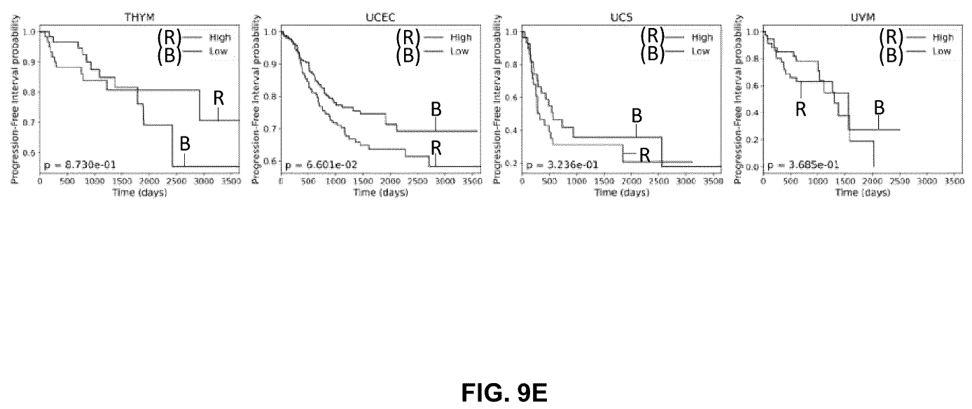

To further substantiate the prognostic utility of the 541-gene RB score, Kaplan-Meier analyses were performed for multiple survival endpoints—overall survival, disease-specific survival, disease-free interval, and progression-free interval—across various TCGA tumor types (FIGS. 6-9). In virtually all cancers assessed, a high RB score (red curves) was consistently associated with worse patient outcomes than a low RB score (blue curves). FIG. 6 shows the detrimental impact of elevated RB activity on overall survival, while FIGS. 7-9 illustrate similarly adverse patterns for disease-specific, disease-free, and progression-free survival, respectively. These results reinforce the notion that tumor cells exhibiting robust ribosome biogenesis gain selective advantages—ranging from rapid proliferation to potential immune evasion—that culminate in an increased likelihood of disease progression and mortality.

Taken together, these analyses establish that the 541-gene RB set not only signifies an essential biological process—ribosome biogenesis—but also provides a powerful, pan-cancer prognostic tool. Its high expression level is consistently linked to poor survival outcomes, implicating ribosome biogenesis as a key driver of malignancy and justifying further efforts to refine and exploit this signature for clinical decision-making.

### Elevated RB Score Is Linked to Aggressive Phenotypes and Immune Modulation in LIHC, KIRC, and LUAD

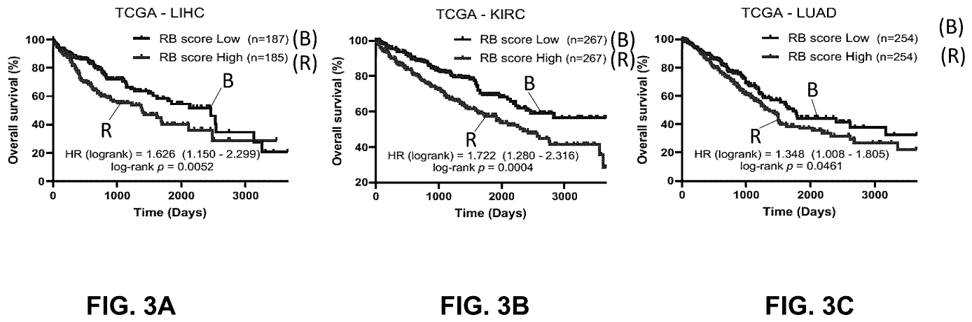

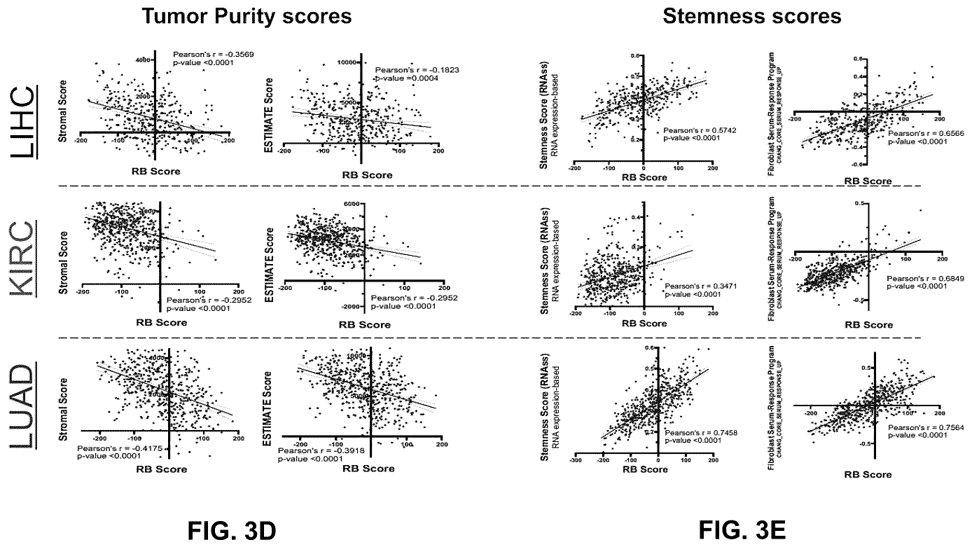

Building on the pan-cancer results, three tumor types were focused on—liver hepatocellular carcinoma (LIHC), kidney renal clear cell carcinoma (KIRC), and lung adenocarcinoma (LUAD)—to investigate how an elevated RB score influences patient outcomes and tumor biology. Across all three cancers, a high RB score (red curves) was significantly associated with worse overall survival, as illustrated by Kaplan-Meier curves in FIGS. 3A-3C (p<0.05 for each comparison). These data suggest that tumors with robust ribosome biogenesis mechanisms exhibit a clinically more aggressive behavior.

Correlation of RB Score with Tumor Purity and Stemness

Further analyses revealed that an increased RB score was inversely correlated with tumor purity. Specifically, Stromal and ESTIMATE scores both tended to be higher when RB scores were lower, indicating that tumors with high RB activity may harbor a denser or more complex microenvironment. Representative scatter plots for LIHC, KIRC, and LUAD confirm these findings in FIG. 3D, where elevated RB scores correlate negatively with these metrics (Pearson's r ranging from −0.18 to −0.42; p<0.0001).

Notably, high RB scores also track closely with increased cancer stemness. As shown in FIG. 3E, multiple stemness indices-such as the RNA expression-based stemness score and fibroblast stemness program-were positively correlated with RB scores (Pearson's r up to ~0.76; p<0.0001). This suggests that heightened ribosome biogenesis may foster an environment conducive to stem-like properties, potentially fueling tumor initiation, therapeutic resistance, and relapse.

Associations with Aneuploidy, Proliferation, and the Immune Microenvironment

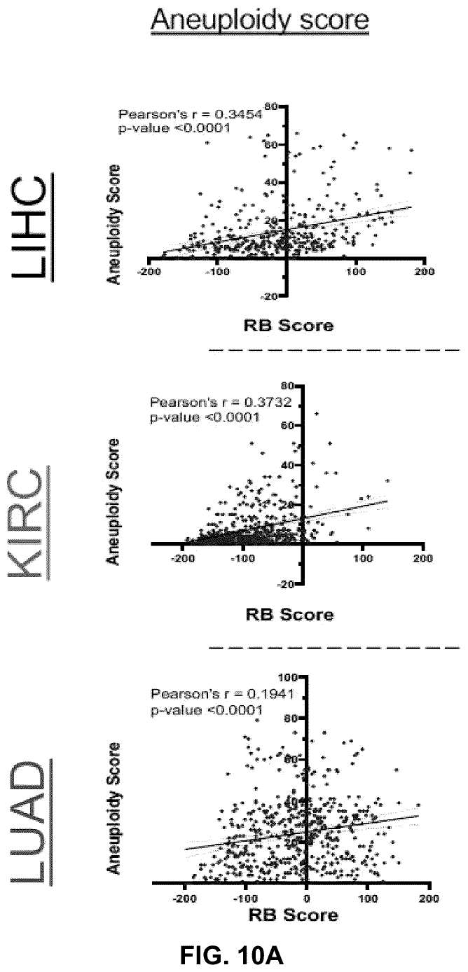

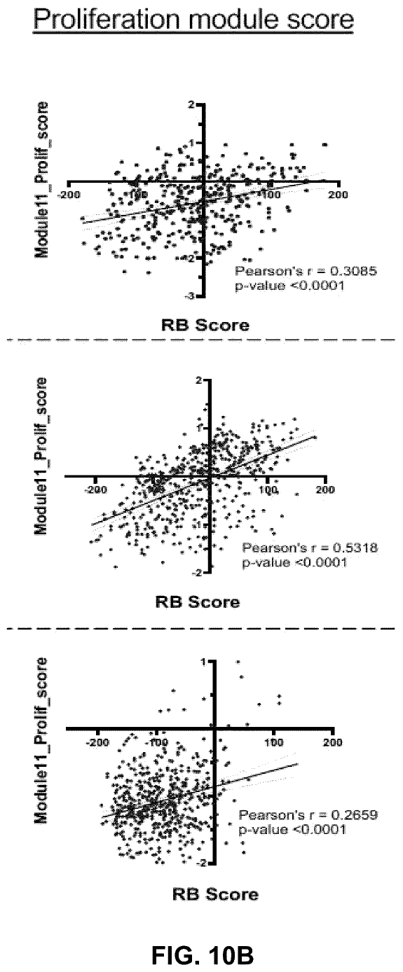

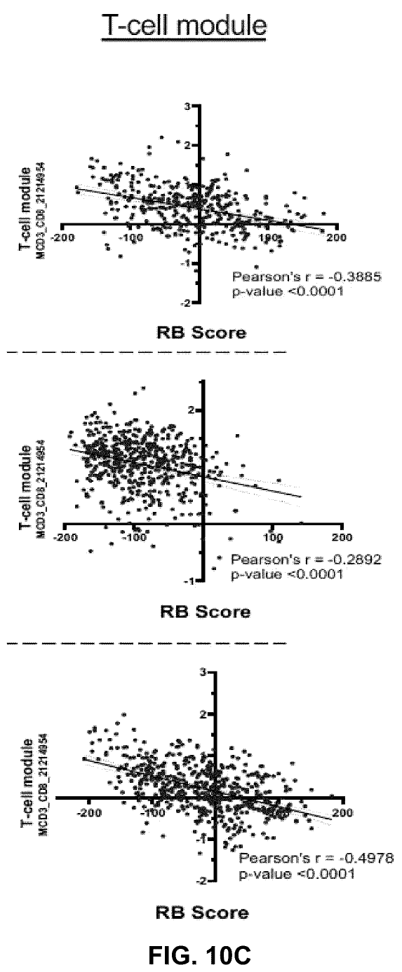

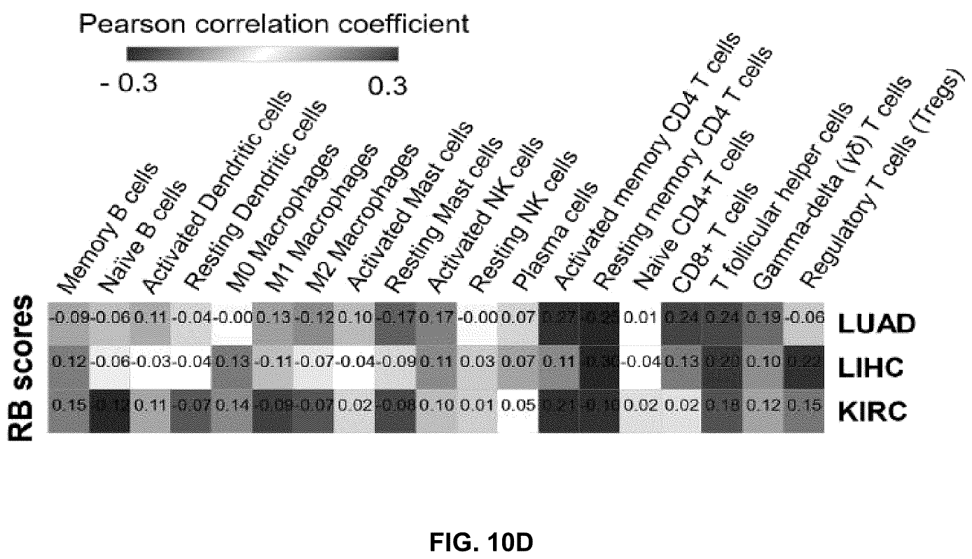

To gain additional insight into the interplay between RB activity and tumor pathophysiology, several key parameters were examined (FIG. 10). First, higher RB scores correlated strongly with aneuploidy (Pearson's r up to 0.73; p<0.0001), implying that dysregulated ribosome biogenesis may coincide with genomic instability in these malignancies. Likewise, the RB score showed a positive link to a proliferation module (Pearson's r up to 0.53; p<0.0001), consistent with a role for ribosomes in supporting rapid tumor growth.

In contrast, an elevated RB score tended to correlate negatively with a T lymphocyte (T-cell) module (Pearson's r down to −0.50; p<0.0001), suggesting that hyperactive ribosome biogenesis may be tied to lower T-cell infiltration or function within the tumor microenvironment. This trend was further corroborated by a heatmap of Pearson correlation coefficients showing varying degrees of positive or negative associations between the RB score and specific immune cell populations (FIG. 10D). Of note, the strongest negative relationships were observed with activated T-cell subsets, raising the possibility that RB-driven mechanisms could impede antitumor immune responses.

Overall, these results reinforce the notion that a high RB score confers multiple features of tumor aggressiveness, including enhanced proliferation, genomic instability, diminished immune infiltration, and elevated stemness. By dissecting these associations in LIHC, KIRC, and LUAD, various embodiments of the present disclosure highlight how upregulated ribosome biogenesis may underlie both intrinsic tumor properties and extrinsic immune-modulatory effects, thereby shaping clinical outcomes in diverse malignancies.

### Development and Validation of the Samant-RB Score in Breast Cancer

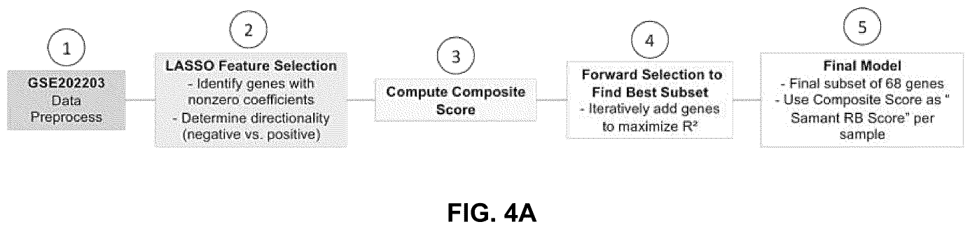

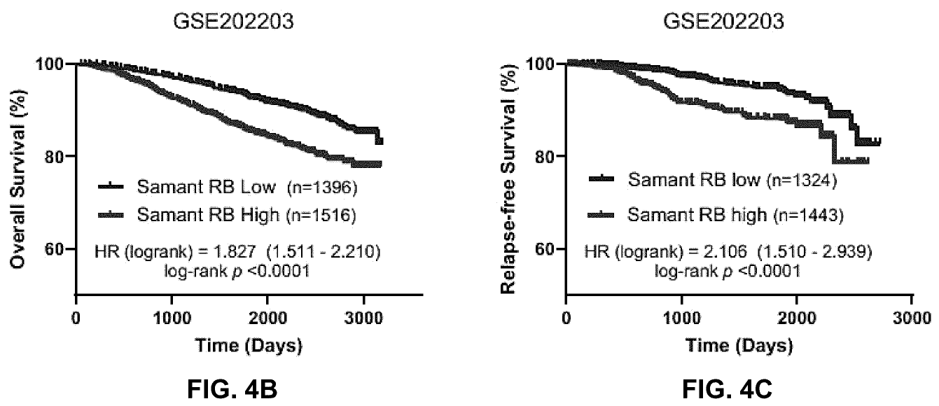

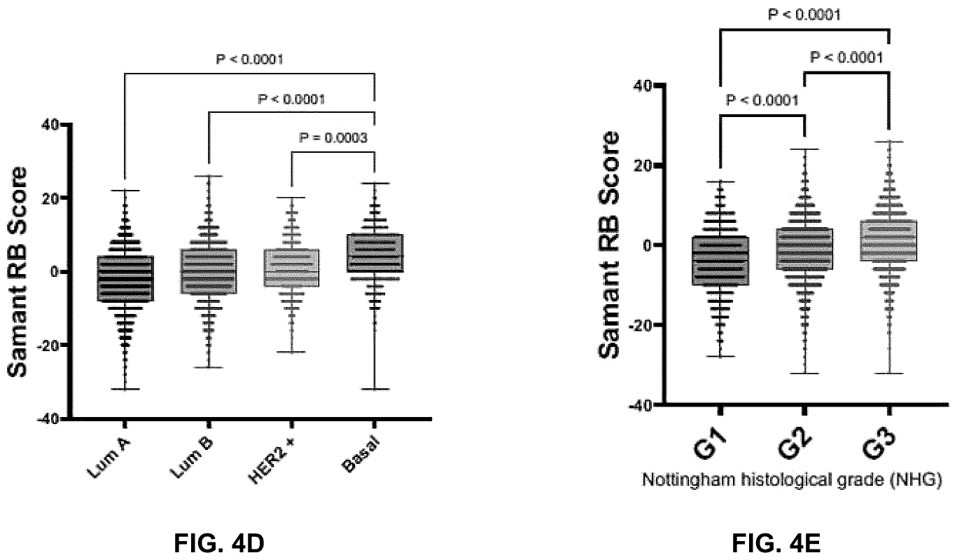

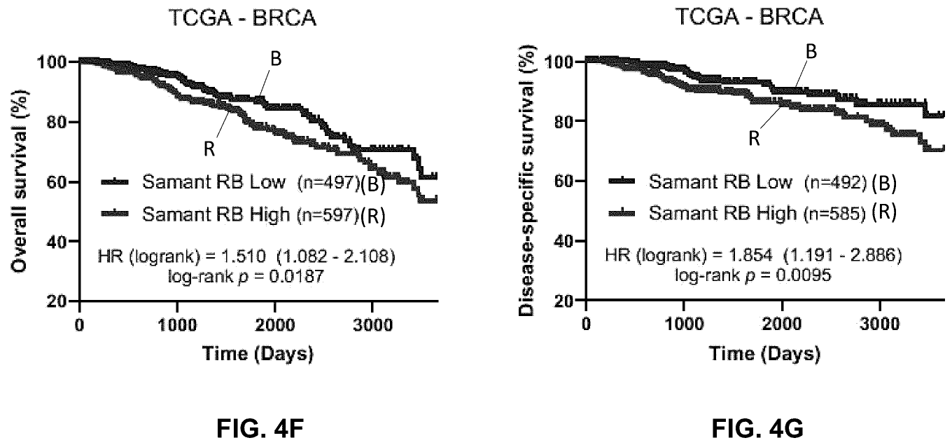

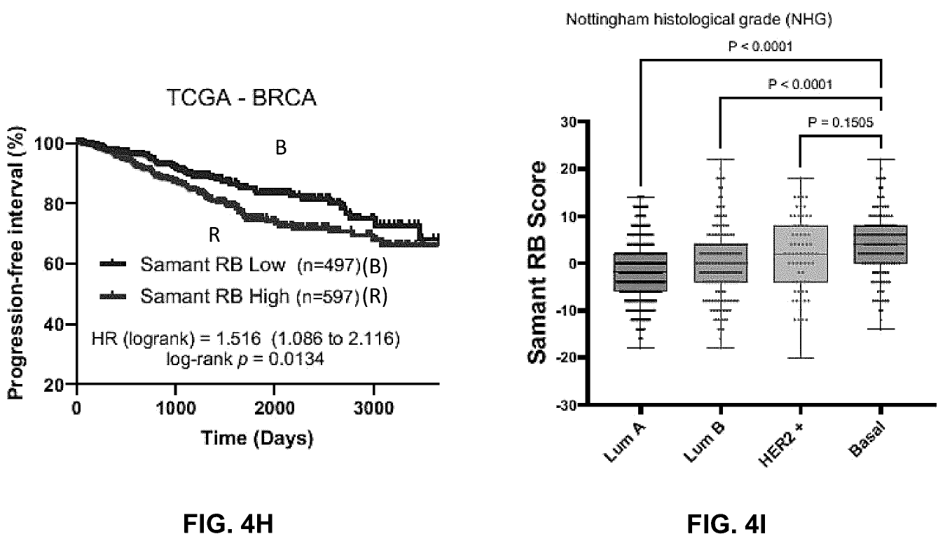

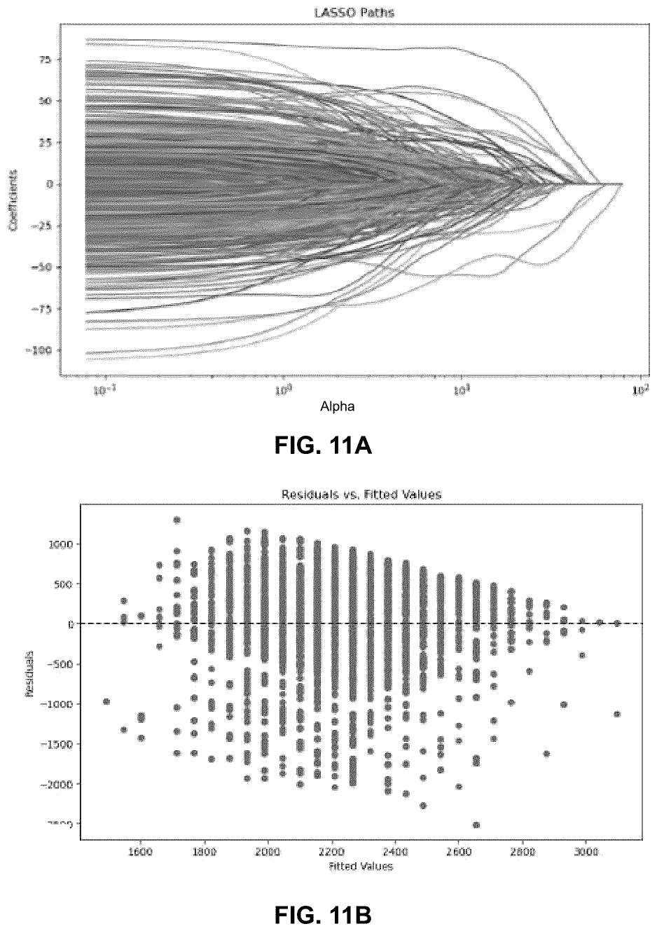

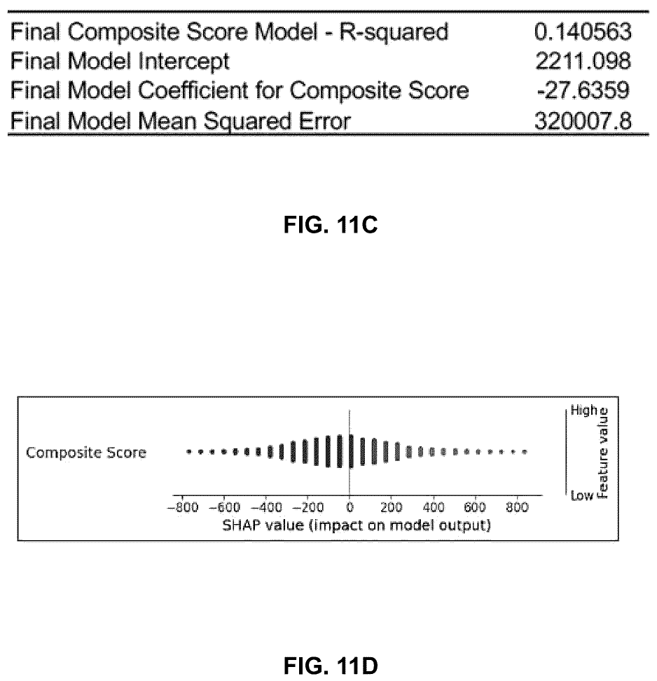

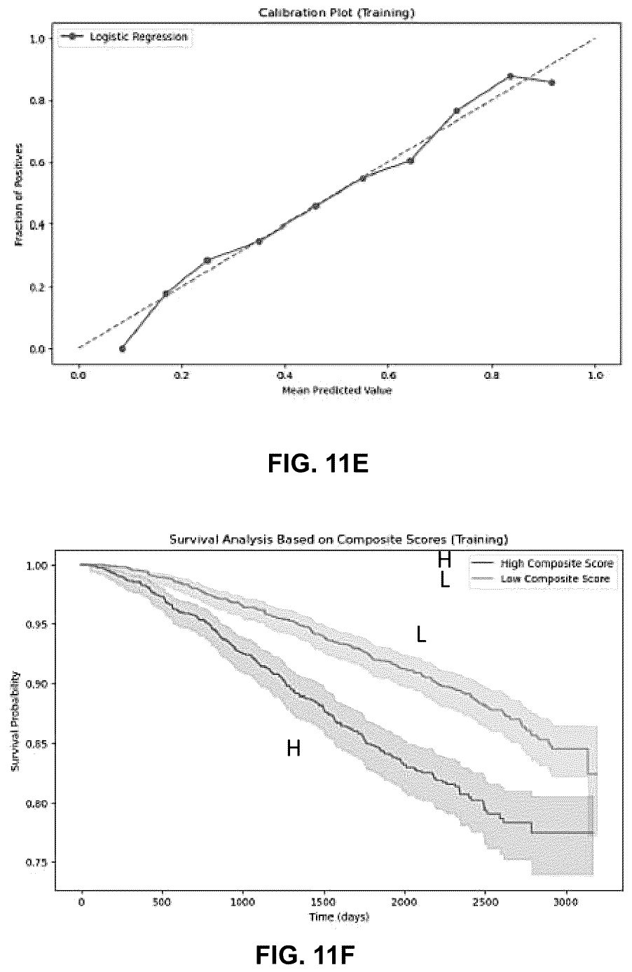

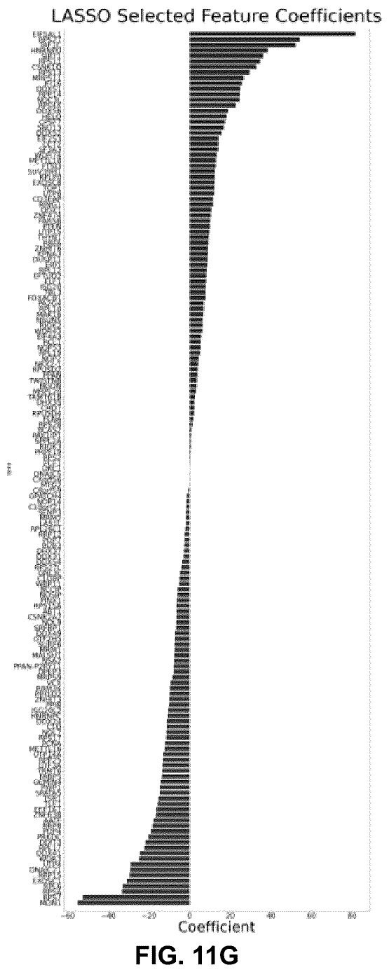

Having established the broad prognostic impact of ribosome biogenesis across multiple solid tumors, breast cancer was focused on to derive a more targeted gene set with enhanced clinical relevance. The GSE202203 cohort was used as a training dataset, employing a LASSO-based feature selection pipeline to narrow the original 541 ribosome biogenesis (RB) genes to a smaller subset (FIG. 4A). Specifically, genes with nonzero LASSO coefficients were first identified and the directionality of each gene's association with survival (negative vs. positive) was determined. A composite ±1 score reflecting whether each gene's expression exceeded or fell below its median expression was then computed, adjusting the sign based on each gene's directionality. A forward selection approach was applied to iteratively optimize the model's explanatory power (coefficient of determination (R²)). This multistep process yielded the final 68-gene Samant-RB Score, whose development is detailed further in FIG. 11 (e.g., LASSO coefficient paths, residual plots, and calibration curves).

### Prognostic Significance in GSE202203

When applied to the GSE202203 cohort, the Samant-RB Score demonstrated robust prognostic capacity. Patients with high Samant-RB Scores (red curves) experienced significantly worse overall survival (HR=1.827, log-rank p<0.0001; FIG. 4B) and relapse-free survival (HR=2.106, log-rank p<0.0001; FIG. 4C). Moreover, the Samant-RB Score varied substantially by breast cancer subtype—with particularly high levels in the more aggressive human epidermal growth factor receptor 2 positive (HER2+) and Basal subtypes—and by Nottingham histological grade (G1 vs. G2 vs. G3), suggesting that upregulated ribosome biogenesis might underlie more advanced tumor phenotypes (FIGS. 4D, 4E).

### Independent Validation in TCGA BRCA

These findings were confirmed in the TCGA breast cancer (BRCA) cohort, where again a high Samant-RB Score correlated with worse overall survival, disease-specific survival, and progression-free interval (FIGS. 4F-4H). Notably, the Basal subtype displayed distinctly elevated Samant-RB Scores relative to Luminal A, Luminal B, and HER2+ subtypes (FIG. 41), reinforcing the notion that enhanced ribosome biogenesis is a hallmark of particularly aggressive breast cancers.

### Functional Insights Into the 68-Gene Signature

To elucidate the biological underpinnings of this refined signature, a protein-protein interaction (PPI) network of the 68 Samant-RB genes was generated in STRING (FIG. 12A). The resultant network exhibited dense clustering, indicating a tightly interlinked set of ribosome biogenesis functions. Furthermore, quartile-based survival analyses in both GSE202203 and TCGA BRCA confirmed the prognostic robustness of the Samant RB Score (FIGS. 12B-12F), with patients in the top quartile of Samant RB expression faring significantly worse than those in the bottom quartile. These data highlight not only the importance of ribosome biogenesis in breast tumor progression but also the practical utility of the Samant-RB Score as an actionable prognostic and potentially predictive biomarker for breast cancer.

### Predictive Potential of the Samant-RB Gene Set in Immunotherapy-Treated Patients

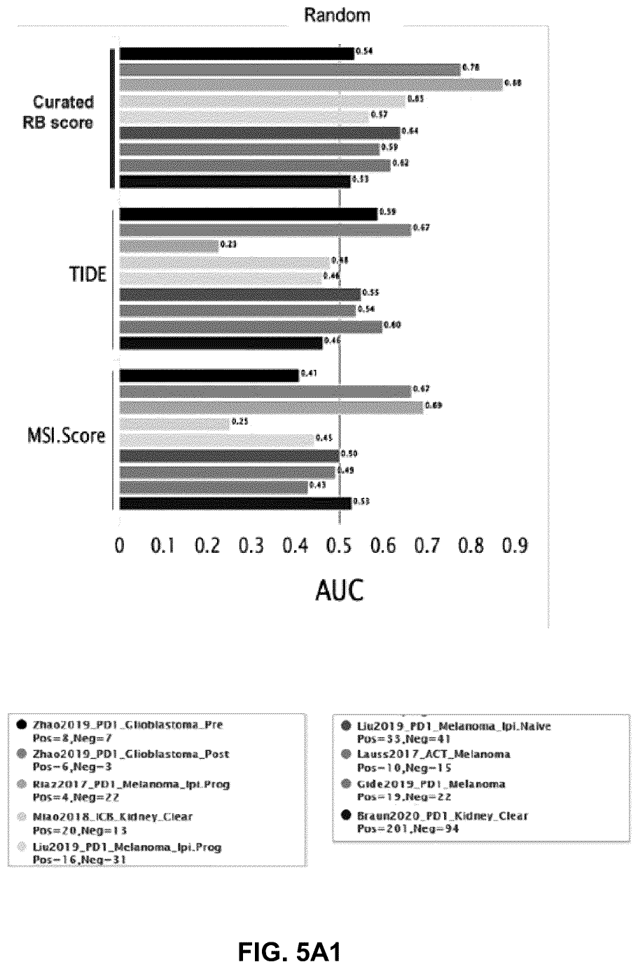

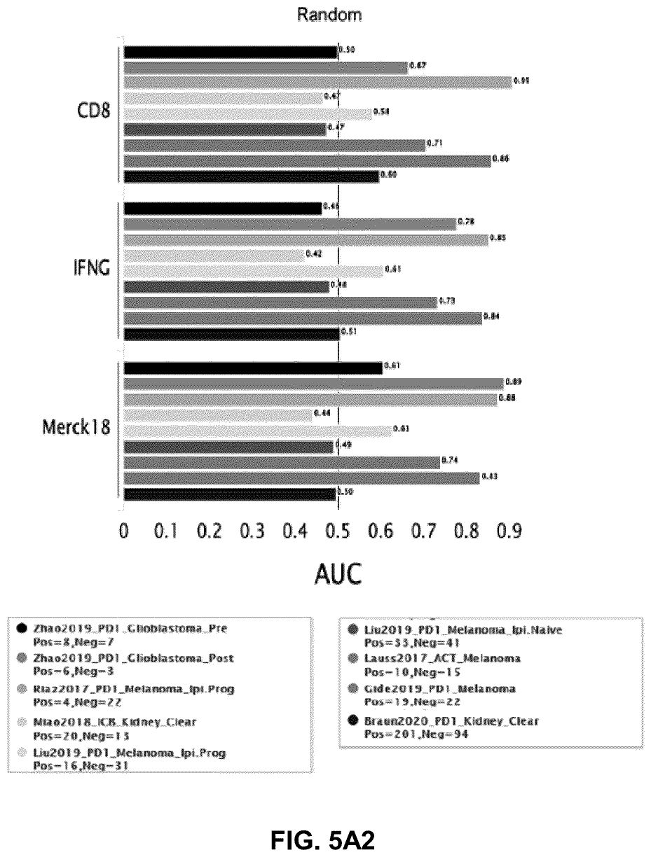

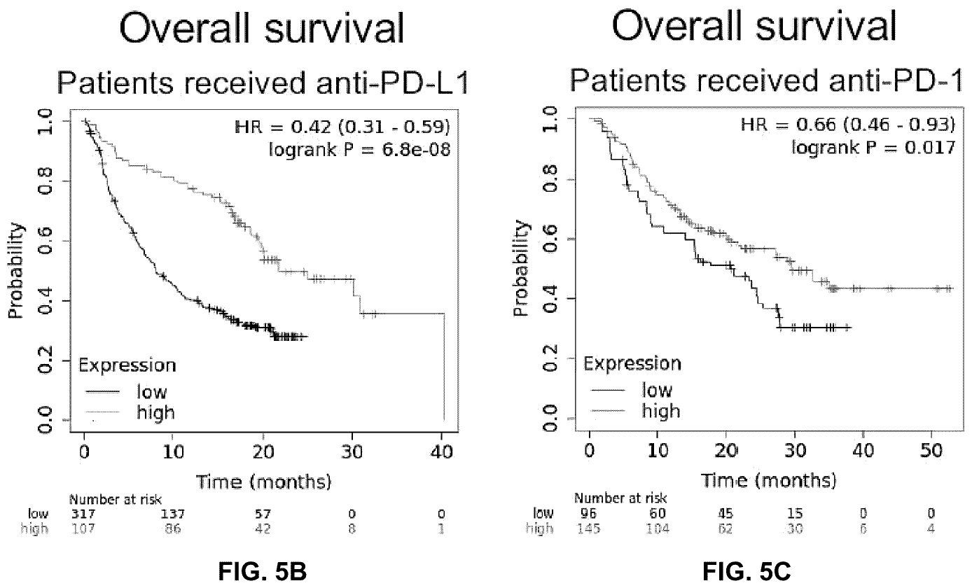

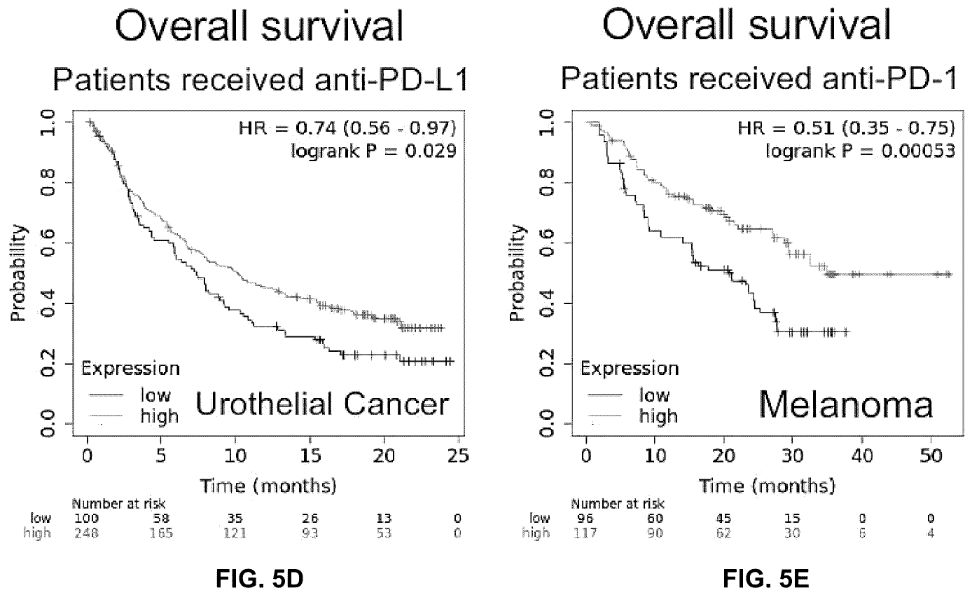

To determine whether heightened ribosome biogenesis also impacts responses to immune checkpoint inhibitors, the Samant-RB Score (or gene expression levels) was compared to established immunotherapy biomarkers using the TIDE platform and KMplot immunotherapy modules. In FIG. 5A, bar plots generated by TIDE illustrate that the curated RB signature (red bar) achieves area under the curve (AUC) values comparable to or exceeding those of well-known biomarkers such as CD8, IFNG, MSI score, and TIDE itself, underscoring its potential predictive value for immunotherapy outcomes. “Random” serves as a baseline reference to highlight the non-random performance of each biomarker.

### Clinical Outcomes in Patients Receiving Anti-PD-1 or Anti-PD-L1

In KMplot analyses (FIG. 5B, 5C), patients with high Samant-RB expression experienced significantly better overall survival (OS) than those with low expression, suggesting that the RB signal can predict benefit—or lack thereof—from anti-PD-L1 or anti-PD-1 therapies. Similar patterns were observed in urothelial cancer and melanoma cohorts (FIG. 5D), where Kaplan-Meier curves again revealed distinct survival trajectories based on RB expression levels. These findings imply that upregulated RB activity may shape the immunotherapy response, possibly by modulating the tumor microenvironment or influencing tumor-immune cell interactions.

### TIDE Analysis Across Multiple Immunotherapy Cohorts

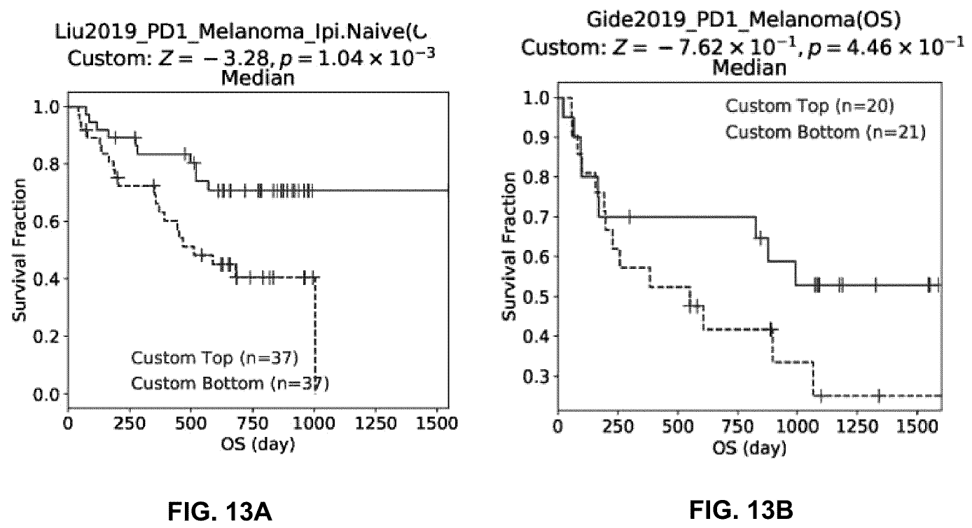

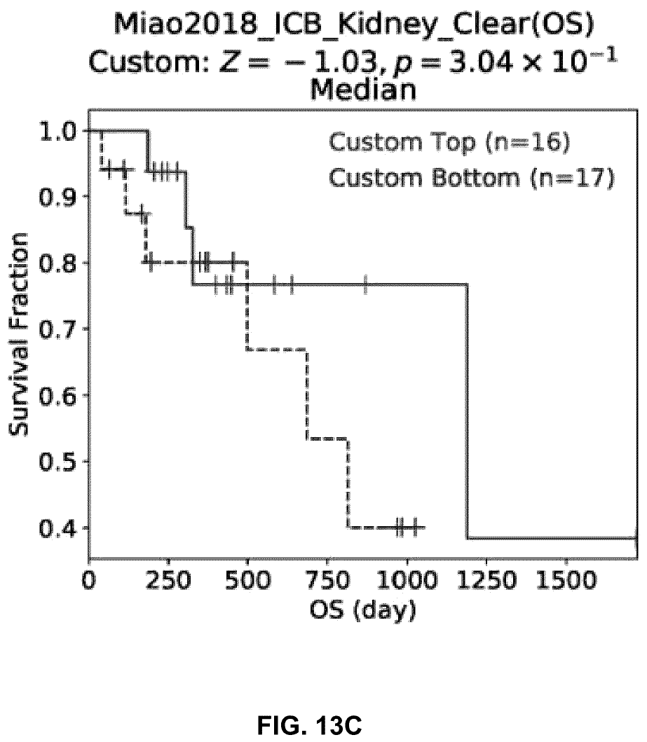

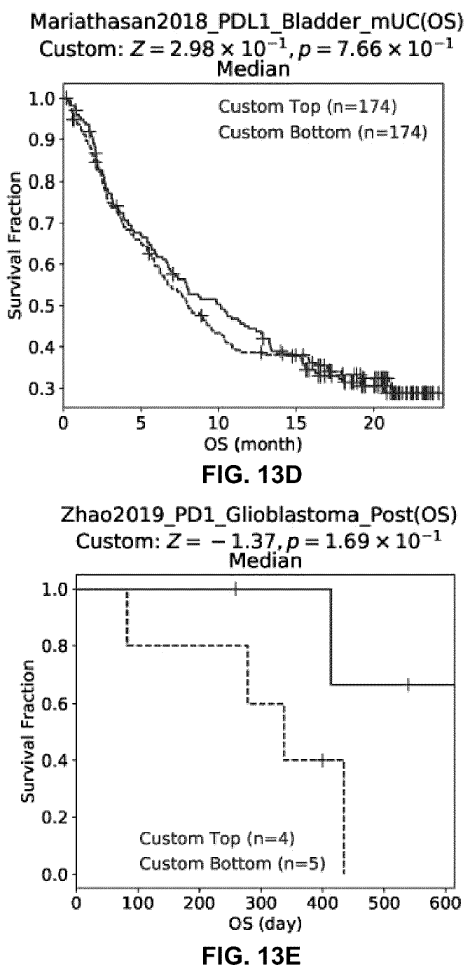

To validate these observations across diverse datasets, additional Kaplan-Meier analyses were performed in FIG. 13, stratifying patients receiving immune checkpoint inhibitors (ICI) by the Samant-RB gene set using TIDE. Across the Liu2019, Gide2019, Miao2018, Mariathasan2018, and Zhao2019 studies, patients displaying elevated Samant-RB expression often exhibited notably better survival outcomes, with Z-statistics and p-values reflecting the log-rank test comparisons (FIGS. 13A-13F). These collective results reinforce the notion that ribosome biogenesis can meaningfully influence ICI efficacy.

In summary, these immunotherapy-focused analyses expand the clinical relevance of ribosome biogenesis beyond prognostication alone. By comparing favorably with established immunotherapy biomarkers and showing strong correlations with OS in multiple ICI-treated cohorts, the Samant-RB signature emerges as a promising candidate for guiding patient selection and optimizing combination treatment strategies in the era of precision oncology.

### Discussion

In this study, a comprehensive investigation into the role of ribosome biogenesis (RB) in cancer was performed, starting with a broad 541-gene set and ultimately refining it into a robust 68-gene “Samant-RB Score.” The findings reveal several key insights. First, the dysregulation of ribosome biogenesis appears to be a pervasive feature across multiple malignancies, as evidenced by the 541 RB genes' prognostic significance in lung, liver, and kidney cancers. Second, the RB score strongly correlates with tumor aggressiveness, immune cell infiltration, aneuploidy, and cancer stemness, suggesting that heightened ribosome biogenesis both supports rapid tumor growth and shapes an immunosuppressive microenvironment. Finally, the development of the Samant-RB Score, specifically in the context of breast cancer, shows that a subset of RB genes can provide refined prognostic stratification and may even predict responsiveness to immunotherapies.

Ribosomes are central to protein synthesis, but recent studies have highlighted their broader involvement in tumorigenesis and metastasis. Consistent with previous evidence, it was found that increased expression of RB genes is linked to shorter overall survival and poorer progression-free outcomes in diverse cancers. This observation supports the hypothesis that hyperactive ribosome biogenesis contributes not only to rapid tumor cell proliferation but also to broader oncogenic processes-such as metabolic reprogramming, stress adaptation, and evasion of immune surveillance. Furthermore, an expanding body of work indicates that oncogenic drivers like MYC and mechanistic target of rapamycin (mTOR) can upregulate ribosomal RNA transcription, while tumor suppressors (e.g., tumor protein p53) often act as “nucleolar guardians” to restrain ribosome production. The pan-cancer findings dovetail with these mechanisms, suggesting that tumors with high RB scores may preferentially leverage these pathways to gain a survival and proliferative advantage.

The data shows that an elevated RB score often inversely correlates with T-cell infiltration and tumor purity, while correlating positively with stemness signatures. These observations align with reports linking ribosome biogenesis to immunomodulatory functions; for instance, high nucleolar activity can modulate the tumor stroma and reduce the effectiveness of cytotoxic T-cells. Additionally, recent studies indicate that cancer stem cells may exploit increased ribosomal output to drive continuous self-renewal and sustain elevated protein synthesis demands during rapid replication. Together, these findings suggest that targeting ribosome biogenesis—perhaps by inhibiting upstream regulators such as RNA polymerase I—may not only blunt tumor growth but also reshape the tumor microenvironment to favor immune effector cells.

While the 541-gene RB set functioned as a robust pan-cancer biomarker, focusing on breast cancer allowed refinement of this large signature into a lean 68-gene “Samant-RB Score” through LASSO-driven feature selection. Notably, high Samant-RB Scores consistently identified patients with poor survival metrics (overall, disease-specific, and relapse-free), highlighting this subset's strong prognostic utility. This aligns with prior work in breast cancer, demonstrating that perturbations in ribosomal RNA modification correlate with tumor grade and subtype. Interestingly, the most aggressive breast subtypes (e.g., basal-like) displayed the highest RB scores, underscoring the potential synergy between RB hyperactivity and molecular features that promote invasiveness and early relapse.

The investigation into multiple immunotherapy cohorts (anti-PD-1 and anti-PD-L1 treatments) revealed that elevated RB expression levels or scores associate with improved therapeutic benefit, as demonstrated by significantly better overall survival in high-RB-score patients across multiple cancer types including non-small cell lung cancer, melanoma, urothelial cancer, glioblastoma, and kidney clear cell carcinoma. Other groups have similarly reported that an immunosuppressive tumor microenvironment can be sculpted, in part, by nucleolar stress responses and ribosome dysfunction. While the precise mechanisms remain to be fully defined, the ability of the Samant-RB Score to rank alongside established biomarkers, including CD8 T-cell and IFNG signatures, positions it as an important candidate for further clinical testing. With immune checkpoint inhibitors at the forefront of cancer therapy, a more nuanced understanding of how RB intersects with immune regulation could guide patient selection or inform combination therapies that jointly target ribosomal pathways and immune checkpoints. Beyond checkpoint inhibition, in examples, the RB score can be extended to predict response to T cell-based therapies including CAR-T, TIL, and TCR-T therapy, and the RB score determined from T cell products prior to infusion can serve as a predictive biomarker of T cell stemness, durability, and therapeutic fitness in adoptive cell therapy settings.

### Conclusions

In summary, this work underscores the pervasive role of RB in shaping cancer progression, emphasizing both proliferative and immunological dimensions. By integrating high-throughput transcriptomic datasets and applying stringent computational selection methods, a refined 68-gene Samant-RB signature was identified that offers robust prognostic power across multiple cohorts-most notably in breast cancer. Additionally, the observed correlations with immunotherapy outcomes suggest that targeting ribosome biogenesis or incorporating the Samant-RB Score into patient stratification may improve the effectiveness of current therapeutic regimens.

### REFERENCES FOR EXAMPLE 1

1. Elhamamsy, A. R., et al., Ribosome Biogenesis: A Central Player in Cancer Metastasis and Therapeutic Resistance. Cancer Res, 2022. 82(13): p. 2344-2353.
2. Pelletier, J., G. Thomas, and S. Volarevic, Ribosome biogenesis in cancer: new players and therapeutic avenues. Nat Rev Cancer, 2018. 18(1): p. 51-63.
3. Jiao, L., et al., Ribosome biogenesis in disease: new players and therapeutic targets. Signal Transduct Target Ther, 2023. 8(1): p. 15.
4. Fuentes, P., J. Pelletier, and A. Gentilella, Decoding ribosome complexity: role of ribosomal proteins in cancer and disease. NAR Cancer, 2024. 6(3): p. zcae032.
5. Dorner, K., et al., Ribosome biogenesis factors—from names to functions. EMBO J, 2023. 42(7): p. e112699.
6. Cui, L., et al., Decoding the ribosome's hidden language: rRNA modifications as key players in cancer dynamics and targeted therapies. Clin Trans1 Med, 2024. 14.(5): p. e1705.
7. Hwang, S. P. and C. Denicourt, The impact of ribosome biogenesis in cancer: from proliferation to metastasis. NAR Cancer, 2024. 6(2): p. zcae017.
8. Jia, X., et al., Protein translation: biological processes and therapeutic strategies for human diseases. Signal Transduct Target Ther, 2024. 9(1): p. 44.
9. Holmberg Olausson, K., M. Nister, and M. S. Lindstrom, p53-Dependent and -Independent Nucleolar Stress Responses. Cells, 2012. 1(4): p. 774-98.
10. Bastide, A. and A. David, The ribosome, (slow) beating heart of cancer (stem) cell. Oncogenesis, 2018. 7(4): p. 34.
11. Ni, C. and M. Buszczak, Ribosome biogenesis and function in development and disease. Development, 2023. 150(5).
12. Gene Ontology, C., et al., The Gene Ontology knowledgebase in 2023. Genetics, 2023. 224(1).
13. Stelzer, G., et al., The GeneCards Suite: From Gene Data Mining to Disease Genome Sequence Analyses. Curr Protoc Bioinformatics, 2016. 54: p. 1 30 1-1 30 33.
14. UniProt, C., UniProt: the Universal Protein Knowledgebase in 2025. Nucleic Acids Res, 2025. 53(D1): p. D609-D617.
15. Amberger, J. S., et al., OMIM.org: leveraging knowledge across phenotype-gene relationships. Nucleic Acids Res, 2019. 47(D1): p. D1038-D1043.
16. Mi, H., et al., PANTHER version 16: a revised family classification, tree-based classification tool, enhancer regions and extensive API. Nucleic Acids Res, 2021. 49(D1): p. D394-D403.
17. Goldman, M. J., et al., Visualizing and interpreting cancer genomics data via the Xena platform. Nat Biotechnol, 2020. 38(6): p. 675-678.
18. Fu, J., et al., Large-scale public data reuse to model immunotherapy response and resistance. Genome Med, 2020. 12(1): p. 21.
19. Kovacs, S. A., J. T. Fekete, and B. Gyorffy, Predictive biomarkers of immunotherapy response with pharmacological applications in solid tumors. Acta Pharmacol Sin, 2023. 44(9): p. 1879-1889.
20. Yoshihara, K., et al., Inferring tumour purity and stromal and immune cell admixture from expression data. Nat Commun, 2013. 4: p. 2612.
21. Zisi, A., J. Bartek, and M. S. Lindstrom, Targeting Ribosome Biogenesis in Cancer: Lessons Learned and Way Forward. Cancers (Basel), 2022. 14(9).
22. Weeks, S. E., B. J. Metge, and R. S. Samant, The nucleolus: a central response hub for the stressors that drive cancer progression. Cell Mol Life Sci, 2019. 76(22): p. 4511-4524.
23. D'Andrea, G., et al., The Paradox of Ribosomal Insufficiency Coupled with Increased Cancer: Shifting the Perspective from the Cancer Cell to the Microenvironment. Cancers (Basel), 2024. 16(13).
24. Zhou, L., et al., T cell proliferation requires ribosomal maturation in nucleolar condensates dependent on DCAF13. J Cell Biol, 2023. 222(10).
25. Ramalho, S., A. Dopler, and W. J. Faller, Ribosome specialization in cancer: a spotlight on ribosomal proteins. NAR Cancer, 2024. 6(3): p. zcae029.
26. Dopler, A., et al., P-stalk ribosomes act as master regulators of cytokine-mediated processes. Cell, 2024. 187(24): p. 6981-6993 e23.
27. Sanchez, C. G., et al., Regulation of Ribosome Biogenesis and Protein Synthesis Controls Germline Stem Cell Differentiation. Cell Stem Cell, 2016. 18(2): p. 276-90.
28. Xue, M., et al., METTL16 promotes liver cancer stem cell self-renewal via controlling ribosome biogenesis and mRNA translation. J Hematol Oncol, 2024. 17(1): p. 7.
29. Zhou, F., et al., A Dynamic rRNA Ribomethylome Drives Stemness in Acute Myeloid Leukemia. Cancer Discov, 2023. 13(2): p. 332-347.
30. Marcel, V., et al., Ribosomal RNA 2′O-methylation as a novel layer of inter-tumour heterogeneity in breast cancer. NAR Cancer, 2020. 2(4): p. zcaa036.
31. Barozzi, C., et al., Alterations of ribosomal RNA pseudouridylation in human breast cancer. NAR Cancer, 2023. 5(2): p. zcad026.
32. Kennecke, H., et al., Metastatic behavior of breast cancer subtypes. J Clin Oncol, 2010. 28(20): p. 3271-7.
33. Ginsburg, O., et al., Breast cancer early detection: A phased approach to implementation. Cancer, 2020. 126 Suppl 10(Suppl 10): p. 2379-2393.
34. Metge, B. J., et al., Ribosomal RNA Biosynthesis Functionally Programs Tumor-Associated Macrophages to Support Breast Cancer Progression. Cancer Res, 2025.
35. Bianco, C. and I. Mohr, Ribosome biogenesis restricts innate immune responses to virus infection and DNA. Elife, 2019. 8.
36. Almeida, L., et al., Ribosome-Targeting Antibiotics Impair T Cell Effector Function and Ameliorate Autoimmunity by Blocking Mitochondrial Protein Synthesis. Immunity, 2021. 54(1): p. 68-83 e6.
37. Chung, S. Y., et al., A G-quadruplex stabilizer, CX-5461 combined with two immune checkpoint inhibitors enhances in vivo therapeutic efficacy by increasing PD-L1 expression in colorectal cancer. Neoplasia, 2023. 35: p. 100856.

### Example 2

### Introduction

### The Importance of Ribosome Biogenesis (RB)

Ribosome biogenesis is a fundamental cellular process essential for protein synthesis. Its regulation is crucial for cellular growth and proliferation, underpinning basic biological functions and disease mechanisms.

### RB Dysregulation in Cancer

In cancer, ribosome biogenesis is often dysregulated, contributing to uncontrolled tumor growth and proliferation. This dysregulation not only meets the increased demand for protein synthesis in rapidly growing cancer cells but also influences tumor-related signaling pathways.

### Research Novelty

Despite its importance, the prognostic implications of ribosome biogenesis dysregulation in cancer remain underexplored. Current therapeutic strategies rarely consider the nuances of ribosome biosynthesis in tumors, overlooking a potential avenue for intervention. Various embodiments of the present disclosure aim to elucidate the prognostic significance of ribosome biogenesis in cancer.

### Methods

### Literature and Database Search

PubMed/MEDLINE, Genecards, Gene Ontology (GO), OMIM, and UniProt were searched up to December 2023 for genes involved in ribosome biogenesis. Pan-cancer TCGA gene expression data was then extracted using the Xena browser.

### Ribosome Biogenesis (RB) Score Calculation

Each gene's expression in a patient's profile was compared against the cohort median. Scores (+1 or −1) were then assigned based on whether expression exceeded or fell below the median. Individual gene scores within the ribosome biogenesis signature were summed to derive the overall RB score for each patient.

### Bioinformatic Analysis

CIBERSORT, ESTIMATE scores, tumor purity, and immune signatures were integrated with RB scores across 32 TCGA tumor types. KM plotter, Morpheus, and GSEA were employed for in-depth analysis.

### Results

FIGS. 1A-1G show identification of an RB gene set and their prognostic value across cancers. FIGS. 3A-3C show high RB score and poor prognosis in different cancers. FIGS. 3D and 3E show RB score is correlated with cancer stemness. FIGS. 10A-10C show RB score is correlated with cancer aneuploidy and proliferation. FIG. 10D shows RB score is correlated with different immune cell populations.

### Conclusion

Various embodiments of the present disclosure have identified a significant correlation between the Ribosome Biogenesis (RB) score and immune cell populations in cancer. This finding highlights the pivotal role of ribosome biogenesis in the tumor immune microenvironment.

### Example 3

Genes of the 68-gene panel described herein (and shown in FIG. 12A; i.e., a plurality of genes described herein from which expression of any two or more or all can be detected or otherwise determined), and the direction of the group/survival prediction of each gene, are shown below:

| | | Group/Survival |
|---|---|---|
| | | Prediction |
| # | Gene Symbol | direction |
| 1 | RPS27 | Positive |
| 2 | RPS7 | Negative |
| 3 | RPS11 | Positive |
| 4 | MDN1 | Negative |
| 5 | TAF1C | Positive |
| 6 | FDXACB1 | Positive |
| 7 | POP4 | Negative |
| 8 | RPS17 | Negative |
| 9 | EIF5AL1 | Positive |
| 10 | GEMIN4 | Negative |
| 11 | CSNK1D | Positive |
| 12 | DDIT3 | Negative |
| 13 | IFI16 | Positive |
| 14 | PWP1 | Negative |
| 15 | DDX1 | Positive |
| 16 | DNAJC21 | Negative |
| 17 | DDX51 | Positive |
| 18 | DDX31 | Negative |
| 19 | EIF2S3 | Positive |
| 20 | C18orf21 | Negative |
| 21 | HNRNPU | Positive |
| 22 | SRFBP1 | Negative |
| 23 | RIOK2 | Positive |
| 24 | MRPS11 | Positive |
| 25 | EXOSC1 | Negative |
| 26 | RCL1 | Positive |
| 27 | UTP14A | Negative |
| 28 | NOC3L | Positive |
| 29 | RPS27L | Negative |
| 30 | ABT1 | Negative |
| 31 | NSUN5 | Positive |
| 32 | POP7 | Negative |
| 33 | MRPL20 | Positive |
| 34 | SUV39H1 | Positive |
| 35 | DDX54 | Negative |
| 36 | PCNA | Negative |
| 37 | CPSF7 | Positive |
| 38 | NOL9 | Negative |
| 39 | TWISTNB | Positive |
| 40 | MALSU1 | Negative |
| 41 | TBL3 | Positive |
| 42 | RRP12 | Negative |
| 43 | NOP2 | Positive |
| 44 | MTG2 | Negative |
| 45 | FTSJ3 | Positive |
| 46 | VCX | Negative |
| 47 | WDR3 | Negative |
| 48 | EXOSC8 | Positive |
| 49 | TLE1 | Negative |
| 50 | RPS15A | Negative |
| 51 | RPS4X | Positive |
| 52 | RPL17 | Negative |
| 53 | RPS28 | Positive |
| 54 | ZNHIT6 | Positive |
| 55 | SPATA5 | Negative |
| 56 | RBFA | Positive |
| 57 | ZNF474 | Positive |
| 58 | TSR1 | Negative |
| 59 | HELQ | Positive |
| 60 | ZNHIT3 | Negative |
| 61 | EIF4A3 | Positive |
| 62 | RPL26L1 | Negative |
| 63 | EEF1A1 | Negative |
| 64 | METTL18 | Positive |
| 65 | RRP15 | Negative |
| 66 | MAK16 | Positive |
| 67 | PINX1 | Negative |
| 68 | RPL19 | Positive |

### Example 4

Genes of the 541-gene panel described herein (as shown in FIG. 2A i.e., a plurality of genes described herein from which expression of any two or more or all can be detected or otherwise determined) comprises the following plurality of genes: AAMP, AATF, ABCE1, ABCF2, ABT1, ACTG1, APEX1, ARPC1A, ARPC4-TTLL3, BCAS2, BCCIP, BLM, BMS1, BOP1, BRIX1, BUB3, BUD23, BYSL, C14orf169, C16orf42, C18orf21, C18orf22, C1D, C1orf107, C1orf25, C1orf66, C1QBP, C20orf132, C21orf57, C21orf70, C22orf28, C4orf43, C6orf153, C7orf30, C8orf59, C9orf114, C9orf98, CBX3, CCAR1, CCDC59, CCT2, CD3EAP, CDC5L, CDCA8, CDK7, CDK9, CDKN2A, CEBPZ, CENPB, CHD7, CIRH1A, COIL, CPSF7, CSNK1D, CSNK1E, CSNK2A2, CSTF1, CUL4A, CUL4B, CXorf56, DCAF13, DDB1, DDIT3, DDX1, DDX10, DDX17, DDX18, DDX19B, DDX21, DDX24, DDX27, DDX28, DDX31, DDX3X, DDX41, DDX42, DDX47, DDX49, DDX5, DDX51, DDX52, DDX54, DDX56, DEK, DHX15, DHX29, DHX30, DHX33, DHX35, DHX37, DHX8, DIMT1, DIMT1L, DIS3, DKC1, DNAJC21, DNAJC5, DNAJC8, DNTTIP2, DOM3Z, DPEP3, DROSHA, DUSP11, DYRK1B, EBNA1BP2, EEF1A1, EEF1A2, EEF1G, EFL1, EFTUD1, EFTUD2, EIF2A, EIF2AK2, EIF2S2, EIF2S3, EIF4A3, EIF5AL1, EIF6, ELF1, EMG1, ERAL1, ERCC2, ERI1, ERI2, ERI3, ESF1, EWSR1, EXOSC1, EXOSC10, EXOSC2, EXOSC3, EXOSC4, EXOSC5, EXOSC6, EXOSC7, EXOSC8, EXOSC9, FABP5, FAM120B, FAM98B, FARSB, FASTKD2, FBL, FBLL1, FCF1, FDXACB1, FEN1, FLNA, FRG1, FTSJ2, FTSJ3, GAR1, GEMIN4, GLTSCR2, GLUL, GNL2, GNL3L, GPATCH4, GRSF1, GSTCD, GTF2H5, GTF3A, GTPBP10, GTPBP4, GTPBP5, HEATR1, HEATR3, HELB, HELQ, HMGN4, HNRNPC, HNRNPD, HNRNPF, HNRNPL, HNRNPR, HNRNPU, HP1BP3, IFI16, ILF3, IMP3, IMP4, IPO4, IPO9, ISG20, ISG20L2, KAT2B, KIAA1239, KIF11, KIF2C, KPNA3, KRI1, KRR1, KRT18, KRT7, LAS1L, LIN9, LSG1, LSM6, LTO1, LTV1, LUC7L2, LYAR, MAF1, MAK16, MALSU1, MDN1, MED21, METT10D, METT5D1, METTL15, METTL15P1, METTL16, METTL17, METTL18, METTL25B, METTL5, MINA, MKI67IP, MPHOSPH10, MPHOSPH6, MPV17L2, MRE11A, MRM1, MRM2, MRM3, MRPL1, MRPL10, MRPL11, MRPL20, MRPL22, MRPL36, MRPS11, MRPS2, MRPS7, MRPS9, MRTO4, MTERF3, MTERF4, MTERFD1, MTERFD2, MTG2, MTREX, MT-RNR1, MT-RNR2, MYBBP1A, MYC, NAF1, NAT10, NCAPG2, NCL, NEDD4, NGDN, NGRN, NHP2, NHP2L1, NIFK, NIP7, NKRF, NKX2-1, NLE1, NMD3, NOB1, NOC2L, NOC3L, NOC4L, NOL10, NOL11, NOL6, NOL7, NOL8, NOL9, NOLC1, NOM1, NOP10, NOP14, NOP16, NOP2, NOP53, NOP56, NOP58, NOP9, NOSIP, NPM1, NPM3, NSA2, NSUN2, NSUN3, NSUN4, NSUN5, NSUN5P1, NSUN5P2, NUDT16, NUMA1, NUP88, NVL, ORAOV1, PA2G4, PAK1IP1, PAPD5, PARN, PCNA, PDCD11, PELP1, PES1, PHF6, PIH1D1, PIH1D2, PIN4, PINX1, PLRG1, PNO1, POLR1A, POLR1B, POLR1C, POLR1D, POLR1E, POLR2E, POLR2F, POLR2H, POLR2K, POLR2L, POP4, POP5, POP7, PPAN, PPAN-P2RY11, PPIB, PRDX1, PRKDC, PRPF19, PRPF3, PRPF8, PTEN, PWP1, PWP2, RAN, RBBP4, RBFA, RBIS, RBM19, RBM34, RBM4B, RCC2, RCL1, REXO4, RFC3, RFC4, RING1, RIOK1, RIOK2, RIOK3, RIOX2, RMRP, RNASEL, RNASEN, RNF40, RNMTL1, RPF1, RPF2, RPL10, RPL10A, RPL10L, RPL11, RPL12, RPL14, RPL17, RPL18, RPL19, RPL23A, RPL24, RPL26, RPL26L1, RPL27, RPL3, RPL35, RPL35A, RPL38, RPL3L, RPL5, RPL6, RPL7, RPL7A, RPL7L1, RPL8, RPLPO, RPLPOP6, RPLP2, RPP14, RPP25, RPP30, RPP38, RPP40, RPS10, RPS11, RPS13, RPS14, RPS15, RPS15A, RPS16, RPS17, RPS19, RPS2, RPS21, RPS24, RPS25, RPS27, RPS27L, RPS28, RPS3, RPS3A, RPS4X, RPS5, RPS6, RPS7, RPS8, RPS9, RPSA, RPUSD1, RPUSD2, RPUSD3, RPUSD4, RRN3, RRP1, RRP12, RRP15, RRP1B, RRP36, RRP7A, RRP7BP, RRP8, RRP9, RRS1, RSL1D1, RSL24D1, RTCD1, RUVBL1, RUVBL2, SART1, SBDS, SDAD1, SENP3, SERBP1, SETX, SF3A3, SF3B14, SFRS1, SFRS3, SFRS5, SHQ1, SIRT1, SIRT7, SKIV2L, SKIV2L2, SLX9, SMC5, SNRNP200, SNRPD2, SNU13, SPATA5, SPPL2A, SPTY2D1, SRFBP1, SRPK1, SUB1, SUPT5H, SUPT6H, SURF6, SUV39H1, TAF1A, TAF1B, TAF1C, TAF1D, TBL3, TBP, TCEB3, TCERG1, TCOF1, TENT4B, TFB1M, TFB2M, THYN1, TLE1, TOP1, TOP2A, TOPBP1, TRAF7, TRMT112, TRMT6, TRMT61B, TSC1, TSR1, TSR2, TSR3, TTF1, TUFM, TWISTNB, UBE21, UBTF, URB1, URB2, USP36, UTP11, UTP11L, UTP14A, UTP14C, UTP15, UTP18, UTP20, UTP23, UTP25, UTP3, UTP4, UTP6, VCX, VRK1, WBP11, WBSCR22, WDR12, WDR18, WDR3, WDR36, WDR43, WDR46, WDR55, WDR74, WDR75, XPO1, XRCC5, XRN2, YBEY, YTHDF2, ZCCHC4, ZCCHC9, ZFC3H1, ZNF474, ZNF622, ZNF638, ZNF658, ZNHIT3, ZNHIT6, ZNRD1.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

At least the following is claimed:

1. A method of identifying a tumor status, the method comprising:
detecting an expression level of two or more of a plurality of genes related to ribosomal biogenesis in one or more samples from a subject, wherein the plurality of genes comprises:
VCX;
DDX1;
SUV39H1;
DNAJC21;
RBFA;
METTL18;
IFI16;
TLE1;
SPATA5;
EIF2S3;
EEF1A1;
MTG2;
PCNA;
FDXACB1;
RPS27;
MRPS11;
EIF5AL1;
MRPL20;
MALSU1;
CSNK1D;
RPS11;
RPS28;
RPS4X;
RPL26L1;
DDX51;
RIOK2;
RPS17;
RPS15A;
RPS27L;
RPS7;
RPL17;
C18orf21;
DDIT3;
RCL1;
SRFBP1;
WDR3;
RPL19;
EXOSC1;
POP4;
TSR1;
RRP12;
UTP14A;
EIF4A3;
POP7;
ZNF474;
TBL3;
NOC3L;
EXOSC8;
TWISTNB;
NOP2;
MAK16;
FTSJ3;
PINX1;
ZNHIT3;
ZNHIT6;
NSUN5;
TAF1C;
PWP1;
DDX31;
DDX54;
ABT1;
GEMIN4;
CPSF7;
HNRNPU;
NOL9;
MDN1;
RRP15; and
HELQ;
assigning one or more values to the expression level of individual ones of the plurality of genes;
summing the one or more assigned values of individual ones of the plurality of genes;
comparing a summed value to a threshold; and
assigning the tumor status of the subject based at least in part on a comparison of the summed value to the threshold.

2. The method of claim 1, wherein the tumor status is an immune infiltration status, a stemness status, a reduced overall survival, or a reduced progression-free interval.

3. The method of claim 1, wherein the subject has or is suspected of having one or more of hepatocellular carcinoma (LIHC), kidney renal clear cell carcinoma (KIRC), a breast cancer, lung adenocarcinoma (LUAD), lung squamous cell carcinoma (LUSC), non-small cell lung cancer (NSCLC), melanoma, urothelial cancer, bladder cancer, glioblastoma (GBM), colorectal cancer, ovarian cancer, stomach adenocarcinoma, or endometrial cancer.

4. The method of claim 1, wherein the method further comprises outputting a report comprising the one or more assigned values, the summed value, the assigned tumor status, or a combination of any thereof.

5. The method of claim 1, wherein the subject is administered a therapy based at least in part on the tumor status assigned to the subject.

6. The method of claim 1, wherein the threshold is a median of the summed value of a plurality of subjects or a quartile of the summed value of the plurality of subjects.

7. A non-transitory, computer-readable medium comprising machine-readable instructions that, when executed by a processor of a computing device, cause the computing device to at least:
detect an expression level of two or more of a plurality of genes related to ribosomal biogenesis in one or more samples from a subject, wherein the plurality of genes comprises:
VCX;
DDX1;
SUV39H1;
DNAJC21;
RBFA;
METTL18;
IFI16;
TLE1;
SPATA5;
EIF2S3;
EEF1A1;
MTG2;
PCNA;
FDXACB1;
RPS27;
MRPS11;
EIF5AL1;
MRPL20;
MALSU1;
CSNK1D;
RPS11;
RPS28;
RPS4X;
RPL26L1;
DDX51;
RIOK2;
RPS17;
RPS15A;
RPS27L;
RPS7;
RPL17;
C18orf21;
DDIT3;
RCL1;
SRFBP1;
WDR3;
RPL19;
EXOSC1;
POP4;
TSR1;
RRP12;
UTP14A;
EIF4A3;
POP7;
ZNF474;
TBL3;
NOC3L;
EXOSC8;
TWISTNB;
NOP2;
MAK16;
FTSJ3;
PINX1;
ZNHIT3;
ZNHIT6;
NSUN5;
TAF1C;
PWP1;
DDX31;
DDX54;
ABT1;
GEMIN4;
CPSF7;
HNRNPU;
NOL9;
MDN1;
RRP15; and
HELQ;
assign one or more values to the expression level of individual ones of the plurality of genes;
sum the one or more assigned values of individual ones of the plurality of genes;
compare a summed value to a threshold; and
assign the tumor status of the subject based at least in part on a comparison of the summed value to the threshold.

8. The non-transitory, computer-readable medium of claim 7, wherein the tumor status is an immune infiltration status, a stemness status, a reduced overall survival, or a reduced progression-free interval.

9. The non-transitory, computer-readable medium of claim 8, wherein the subject has or is suspected of having one or more of hepatocellular carcinoma (LIHC), kidney renal clear cell carcinoma (KIRC), a breast cancer, lung adenocarcinoma (LUAD), lung squamous cell carcinoma (LUSC), non-small cell lung cancer (NSCLC), melanoma, urothelial cancer, bladder cancer, glioblastoma (GBM), colorectal cancer, ovarian cancer, stomach adenocarcinoma, or endometrial cancer.

10. The non-transitory, computer-readable medium of claim 8, wherein the machine-readable instructions, when executed by the processor, further cause the computing device to at least output a report comprising the one or more assigned values, the summed value, the assigned tumor status, or a combination of any thereof.

11. The non-transitory, computer-readable medium of claim 8, wherein the threshold is a median of the summed value of a plurality of subjects or a quartile of the summed value of the plurality of subjects.

12. A kit for determining a tumor status in one or more samples from a subject, comprising:
a plurality of primer pairs for determining expression levels of one or more of a plurality of genes of claim 1; and
instructions for use.

13. A system for determining a tumor status in one or more samples from a subject, comprising:
a computing device comprising a processor and a memory; and
machine-readable instructions stored in the memory that, when executed by the processor, cause the computing device to at least:
identify an expression level of two or more of a plurality of genes related to ribosomal biogenesis in the one or more samples from the subject, wherein the plurality of genes comprises:
VCX;
DDX1;
SUV39H1;
DNAJC21;
RBFA;
METTL18;
IFI16;
TLE1;
SPATA5;
EIF2S3;
EEF1A1;
MTG2;
PCNA;
FDXACB1;
RPS27;
MRPS11;
EIF5AL1;
MRPL20;
MALSU1;
CSNK1D;
RPS11;
RPS28;
RPS4X;
RPL26L1;
DDX51;
RIOK2;
RPS17;
RPS15A;
RPS27L;
RPS7;
RPL17;
C18orf21;
DDIT3;
RCL1;
SRFBP1;
WDR3;
RPL19;
EXOSC1;
POP4;
TSR1;
RRP12;
UTP14A;
EIF4A3;
POP7;
ZNF474;
TBL3;
NOC3L;
EXOSC8;
TWISTNB;
NOP2;
MAK16;
FTSJ3;
PINX1;
ZNHIT3;
ZNHIT6;
NSUN5;
TAF1C;
PWP1;
DDX31;
DDX54;
ABT1;
GEMIN4;
CPSF7;
HNRNPU;
NOL9;
MDN1;
RRP15; and
HELQ;
assign one or more values to the expression level of individual ones of the plurality of genes;
sum the one or more assigned values of individual ones of the plurality of genes;
compare a summed value to a threshold; and
assign the tumor status of the subject based at least in part on a comparison of the summed value to the threshold.

14. The system of claim 13, wherein the tumor status is an immune infiltration status, a stemness status, a reduced overall survival, or a reduced progression-free interval.

15. The system of claim 14, wherein the subject has or is suspected of having one or more of hepatocellular carcinoma (LIHC), kidney renal clear cell carcinoma (KIRC), a breast cancer, lung adenocarcinoma (LUAD), lung squamous cell carcinoma (LUSC), non-small cell lung cancer (NSCLC), melanoma, urothelial cancer, bladder cancer, glioblastoma (GBM), colorectal cancer, ovarian cancer, stomach adenocarcinoma, or endometrial cancer.

16. The system of claim 14, wherein the machine-readable instructions, when executed by the processor, further cause the computing device to at least output a report comprising the one or more assigned values, the summed value, the assigned tumor status, or a combination of any thereof.

17. The system of claim 14, wherein the subject is administered a therapy based at least in part on the tumor status assigned to the subject.

18. The system of claim 14, wherein the threshold is a median of the summed value of a plurality of subjects or a quartile of the summed value of the plurality of subjects.

19. The system of claim 14, wherein the machine-readable instructions that, when executed by the processor, cause the computing device to identify the expression level further cause the computing device to at least:
receive expression data from a thermal cycler and an optical detection device; and
identify the expression level of one or more of the plurality of genes related to ribosomal biogenesis in the one or more samples from the subject based at least in part on the expression data.

20. The system of claim 14, further comprising a thermal cycler and an optical detection device.
